SEQUENCE LISTING (34 sequences)

SEQ ID NO: 1 - protein, 163 aa, Chlamydomonas reinhardtii
MNTKYNKEFLLYLAGFVDGDGSIIAQIKPNQSYKFKHQLSLAFQVTQKTQRRWFLDKLVDEIGVGYVRDRGSVSDYILSEIKPLHNFLTQLQPFLKLKQKQANLVLKIIWRLPSAKESPDKFLEVCTWVDQIAALNDSKTRKTTSETVRAVLDSLSEKKKSSP

SEQ ID NO: 2 - protein, 354 aa, Artificial Sequence
  Synthesized
MNTKYNKEFLLYLAGFVDGDGSIIAQIKPNQSYKFKHQLSLAFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIIAQIKPNQSYKFKHQLSLAFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 3 - DNA, 24 bp, Artificial Sequence
  Synthesized
gataaggattgtaagacttcatcc

SEQ ID NO: 4 - protein, 354 aa, Artificial Sequence
  Synthesized
MNTKYNKEFLLYLAGFVDGDGSIYATIMPNQRAKFRHVLRLHFNVSQKTQRRWFLDKLVDEIGVGYVRDRGSVSDYVLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSITATITPCQRVKFKHYLQLRFNVSQKTQRRWFLDKLVDEIGVGYVQDLGSVSEYRLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 5 - DNA, 1062 bp, Artificial Sequence
  Synthesized
atgaatacaaaatataataaagagttcttactctacttagcagggtttgtagacggtgacggttccatctatgccactatcatgcctaatcaacgggctaagttcaggcacgttctgcggctccatttcaatgtctctcagaagacacagcgccgttggttcctcgacaagctggtggacgagatcggtgtgggttacgtgcgggaccgtggcagcgtctccgattacgtgctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatcacggccactatcacgccttgtcaacgtgtgaagttcaagcactatctgcagctccgtttcaatgtctctcagaagacacagcgccgttggttcctcgacaagctggtggacgagatcggtgtgggttacgtgcaggacctgggcagcgtctccgagtaccgtctgtcccagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccc

SEQ ID NO: 6 - protein, 35 aa, Homo sapiens
MSVLTPLLLRGLTGSARRLPVPRAKIHSLPPEGKL

SEQ ID NO: 7 - protein, 63 aa, Neurospora crassa
MASTRVLASRLASQMAASAKVARPAVRVAQVSKRTIQTGSPLQTLKRTQMTSIVNATTRQAFQ

SEQ ID NO: 8 - protein, 98 aa, Artificial Sequence
  Synthesized
MSVLTPLLLRGLTGSARRLPVPRAKIHSLPPEGKLMASTRVLASRLASQMAASAKVARPAVRVAQVSKRTIQTGSPLQTLKRTQMTSIVNATTRQAFQ

SEQ ID NO: 9 - protein, 18 aa, Mouse parovirus NS2
VDEMTKKFGTLTIHDTEK

SEQ ID NO: 10 - protein, 10 aa, Artificial Sequence
  Synthesized
LGAGLGALGL

SEQ ID NO: 11 - DNA, 24 bp, Artificial Sequence
  Synthesized
ctattcctaacattctgaagtagg

SEQ ID NO: 12 - DNA, 23 bp, Artificial Sequence
  Synthesized
ccaccctagctatcataagcaca

SEQ ID NO: 13 - DNA, 44 bp, Artificial Sequence
  Synthesized
aagcaattgatttgcattcaatagatgtaggatgaagtcctgca

SEQ ID NO: 14 - DNA, 20 bp, Artificial Sequence
  Synthesized
gcctgacccatagccataat

SEQ ID NO: 15 - DNA, 20 bp, Artificial Sequence
  Synthesized
cggctgcgtattctacgtta

SEQ ID NO: 16 - DNA, 25 bp, Artificial Sequence
  Synthesized
tctcaaccctagcagaaacaaccgg

SEQ ID NO: 17 - DNA, 23 bp, Artificial Sequence
  Synthesized
cccatgactaccatcagcaatag

SEQ ID NO: 18 - DNA, 20 bp, Artificial Sequence
  Synthesized
tggaatcggaccagtaggaa

SEQ ID NO: 19 - DNA, 21 bp, Artificial Sequence
  Synthesized
agtgctgaactggtgtagggc

SEQ ID NO: 20 - DNA, 20 bp, Artificial Sequence
  Synthesized
gccgctagaggtgaaattct

SEQ ID NO: 21 - DNA, 20 bp, Artificial Sequence
  Synthesized
tcggaactacgacggtatct

SEQ ID NO: 22 - DNA, 23 bp, Artificial Sequence
  Synthesized
aagacggaccagagcgaaagcat

SEQ ID NO: 23 - DNA, 21 bp, Artificial Sequence
  Synthesized
gacttcatcctacatctattg

SEQ ID NO: 24 - DNA, 22 bp, Artificial Sequence
  Synthesized
gtaaggatagtaagtcttccaa

SEQ ID NO: 25 - DNA, 22 bp, Artificial Sequence
  Synthesized
tcaaggatagcacgtctccaac

SEQ ID NO: 26 - DNA, 22 bp, Artificial Sequence
  Synthesized
ttaaggatggtaagacagtatc

SEQ ID NO: 27 - DNA, 22 bp, Artificial Sequence
  Synthesized
ctaaggttagtaagtattcaac

SEQ ID NO: 28 - DNA, 22 bp, Artificial Sequence
  Synthesized
ttcaggatggcacgtcttcatc

SEQ ID NO: 29 - DNA, 20 bp, Artificial Sequence
  Synthesized
agccccgggtactccttgtt

SEQ ID NO: 30 - DNA, 18 bp, Artificial Sequence
  Synthesized
ttccttgcaggaacagag

SEQ ID NO: 31 - DNA, 17 bp, Artificial Sequence
  Synthesized
ctgcttgaccacccatt

SEQ ID NO: 32 - DNA, 19 bp, Artificial Sequence
  Synthesized
ccagcaggccaggtacacc

SEQ ID NO: 33 - DNA, 16 bp, Artificial Sequence
  Synthesized
accgccaaggatgcac

SEQ ID NO: 34 - DNA, 16 bp, Artificial Sequence
  Synthesized
gcgggtgggaatggag

(19) United States
(12) Patent Application Publication
(10) Pub. No.: US 2024/0200046 A1
(43) Pub. Date: Jun. 20, 2024

(54) ENGINEERED MEGANUCLEASES THAT TARGET HUMAN MITOCHONDRIAL GENOMES

(71) Applicants: Precision BioSciences, Inc., Durham, NC (US); NORTH CAROLINA STATE UNIVERSITY, Raleigh, NC (US)

(72) Inventors: Carlos T. Moraes, Miami, FL (US); James Jefferson Smith, Morrisville, NC (US); Derek Jantz, Durham, NC (US); Ginger H. Tomberlin, Cary, NC (US); John Morris, Raleigh, NC (US); Wendy Shoop, Durham, NC (US)

(73) Assignees: Precision BioSciences, Inc., Durham, NC (US); University of Miami, Miami, FL (US)

(21) Appl. No.: 18/556,593

(22) PCT Filed: Apr. 22, 2022
(86) PCT No.: PCT/US2022/025945

§ 371 Date: Oct. 20, 2023

Related U.S. Application Data
(60) Provisional application No. 63/178,269, filed on Apr. 22, 2021.

(51) Int. Cl. C12N 9/22; A61K 9/127; A61K 38/46; A61K 48/00; C12N 15/86

(52) U.S. Cl. CPC C12N 9/22; A61K 9/127; A61K 38/465; A61K 48/005; C12N 15/86; C07K 2319/07; C12N 2750/14143

(57) ABSTRACT

Disclosed herein are recombinant meganucleases engineered to recognize and cleave a recognition sequence present in the human mitochondrial DNA (mtDNA). The disclosure further relates to the use of such recombinant meganucleases in combination with mitochondrial transit peptides in methods for producing genetically-modified eukaryotic cells, and to a population of genetically-modified eukaryotic cells wherein the mtDNA has been modified or edited.

### FIELD OF THE INVENTION

The present disclosure relates to the field of molecular biology and recombinant nucleic acid technology. In particular, the present disclosure relates to recombinant meganucleases engineered to recognize and cleave recognition sequences found in the human mitochondrial genome. The present disclosure further relates to the use of such recombinant meganucleases in methods for producing genetically-modified eukaryotic cells, and to a population of genetically-modified eukaryotic cells wherein the mitochondrial DNA has been modified.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Apr. 21, 2022 is named P89339_0137_9_SeqList_4-21-22.txt and is 20.0 kb in size.

### BACKGROUND OF THE INVENTION

In all organisms, mitochondria regulate cellular energy and metabolism under normal growth and development as well as in response to stress. Many of the proteins functioning in these roles are coded for in the mitochondrial genome. Thus, editing of the mitochondrial genome has diverse applications in both animals and plants. In humans, deleterious mitochondrial mutations are the source of a number of disorders for which gene editing therapies could be applied.

Pathogenic mitochondrial DNA (mtDNA) mutations include large-scale rearrangements and point mutations in protein coding, transfer RNA (tRNA) or ribosomal RNA (rRNA) genes. Although the prevalence of mtDNA-related disease diagnosis is about 1 in 5,000, the population frequency of the ten most common pathogenic mtDNA mutations is much higher, approaching 1 in 200, implying that many “normal” individuals carry low levels of mutated genomes (Schon et al., Nat Rev Gen 13:878-890 (2012)).

Mutated mtDNA, in most cases, co-exist with wild-type mtDNA in patients' cells (mtDNA heteroplasmy). Several studies showed that the wild-type mtDNA has a strong protective effect, and biochemical abnormalities were observed only when the levels of the mutated mtDNA were higher than 80-90% (Schon et al., Nature Reviews Genetics 13:878-890 (2012)). It has been shown that muscle fibers develop an OXPHOS defect only when the mutation load is above 80% (Sciacco et al., Hum Mol Genet 3:13-19 (1994)). Therefore, any approach that could shift this balance by even a small percentage towards the wild-type would have strong therapeutic potential.

However, mtDNA manipulation remains an underexplored area of science because of the inability to target mtDNA at high efficiencies and generate precise edits. The mitochondrial genome is difficult to edit because it requires predictable repair mechanisms and delivery of an editing technology to this organelle. In view of the difficulty and unpredictability associated with mitochondrial genome editing, there is an unmet need for precise editing of mtDNA, which would open up an entire field of inquiry and opportunity in life sciences. The ability to target and edit a defined region (preferably limited to just one gene) of the mitochondrial genome in a more predictable manner would be a clear benefit over currently available systems.

### SUMMARY OF THE INVENTION

Provided herein are compositions and methods for precise editing of mitochondrial genome. Up until now, all other attempts at mitochondrial genome editing have resulted in large and unpredictable deletions/rearrangements. The present invention demonstrates for the first time that homing endonucleases allow precise editing of mitochondrial DNA (mtDNA), thereby opening up an entire field of inquiry and opportunity in life sciences. The compositions and methods provided herein can be used for editing one specific mitochondrial gene without impacting surrounding regions.

In one aspect, the invention provides a mitochondria-targeting engineered meganuclease (MTEM) that binds and cleaves a recognition sequence in mitochondrial genomes of a eukaryotic cell, wherein the MTEM comprises an engineered meganuclease attached to a mitochondrial transit peptide (MTP).

In some embodiments, the engineered meganuclease comprises a first subunit and a second subunit, wherein the first subunit binds to a first recognition half-site of the recognition sequence and comprises a first hypervariable (HVR1) region, the second subunit binds to a second recognition half-site of the recognition sequence and comprises a second hypervariable (HVR2) region, and the first subunit and the second subunit each comprise an amino acid sequence having at least 80%, 85%, 90%, 91%, 92%, 93%, 94%, 95%, 96%, 97%, 98%, 99% or more sequence identity to a sequence set forth in SEQ ID NO: 1. In some embodiments, the first subunit and the second subunit each comprise an amino acid sequence having at least 80%, 85%, 90%, 91%, 92%, 93%, 94%, 95%, 96%, 97%, 98%, 99% or more sequence identity to residues 7-153 of SEQ ID NO: 1. In some embodiments, the engineered meganuclease comprises an amino acid sequence having at least 80%, 85%, 90%, 91%, 92%, 93%, 94%, 95%, 96%, 97%, 98%, 99% or more sequence identity to a sequence set forth in SEQ ID NO: 2.

In some embodiments, the recognition sequence comprises SEQ ID NO: 3.

In some embodiments, the HVR1 region comprises an amino acid sequence having at least 80%, 85%, 90%, 91%, 92%, 93%, 94%, 95%, 96%, 97%, 98%, 99% or more sequence identity to an amino acid sequence corresponding to residues 215-270 of SEQ ID NO: 4. In some embodiments, the HVR1 region comprises one or more residues corresponding to residues 215, 217, 219, 221, 223, 224, 229, 231, 233, 235, 237, 259, 261, 266, and 268 of SEQ ID NO: 4. In some embodiments, the HVR1 region comprises residues corresponding to residues 215, 217, 219, 221, 223, 224, 229, 231, 233, 235, 237, 259, 261, 266, and 268 of SEQ ID NO: 4. In some embodiments, the HVR1 region comprises Y, R, K, or D at a residue corresponding to residue 257 of SEQ ID NO: 4. In some embodiments, the HVR1 region comprises residues 215-270 of SEQ ID NO: 4 with up to 1, 2, 3, 4, 5, 6, 7, 8, 9, 10, or 11 amino acid substitutions. In some embodiments, the HVR1 region comprises residues 215-270 of SEQ ID NO: 4.

In some embodiments, the first subunit comprises an amino acid sequence having at least 80%, 85%, 90%, 91%, 92%, 93%, 94%, 95%, 96%, 97%, 98%, 99% or more sequence identity to residues 198-344 of SEQ ID NO: 4. In some embodiments, the first subunit comprises a residue corresponding to residue 271 of SEQ ID NO: 4. In some embodiments, the first subunit comprises G, S, or A at a residue corresponding to residue 210 of SEQ ID NO: 4. In some embodiments, the first subunit comprises E, Q, or K at a residue corresponding to residue 271 of SEQ ID NO: 4. In some embodiments, the first subunit comprises residues 198-344 of SEQ ID NO: 4 with up to 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid substitutions. In some embodiments, the first subunit comprises residues 198-344 of SEQ ID NO: 4.

In some embodiments, the HVR2 region comprises an amino acid sequence having at least 80%, 85%, 90%, 91%, 92%, 93%, 94%, 95%, 96%, 97%, 98%, 99% or more sequence identity to an amino acid sequence corresponding to residues 24-79 of SEQ ID NO: 4. In some embodiments, the HVR2 region comprises one or more residues corresponding to residues 24, 26, 28, 30, 32, 33, 38, 40, 42, 44, 46, 68, 70, 75, and 77 of SEQ ID NO: 4. In some embodiments, the HVR2 region comprises residues corresponding to residues 24, 26, 28, 30, 32, 33, 38, 40, 42, 44, 46, 68, 70, 75, and 77 of SEQ ID NO: 4. In some embodiments, the HVR2 region comprises a residue corresponding to residue 36 of SEQ ID NO: 4. In some embodiments, the HVR2 region comprises Y, R, K, or D at a residue corresponding to residue 66 of SEQ ID NO: 4. In some embodiments, the HVR2 region comprises residues 24-79 of SEQ ID NO: 4 with up to 1, 2, 3, 4, 5, 6, 7, 8, 9, 10, or 11 amino acid substitutions. In some embodiments, the HVR2 region comprises residues 24-79 of SEQ ID NO: 4.

In some embodiments, the second subunit comprises an amino acid sequence having at least 80%, 85%, 90%, 91%, 92%, 93%, 94%, 95%, 96%, 97%, 98%, 99% or more sequence identity to residues 7-153 of SEQ ID NO: 4. In some embodiments, the second subunit comprises residues 7-153 of SEQ ID NO: 4 with up to 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid substitutions. In some embodiments, the second subunit comprises G, S, or A at a residue corresponding to residue 19 of SEQ ID NO: 4. In some embodiments, the second subunit comprises E, Q, or K at a residue corresponding to residue 80 of SEQ ID NO: 4. In some embodiments, the second subunit comprises residues 7-153 of SEQ ID NO: 4.

In some embodiments, the engineered meganuclease is a single-chain meganuclease comprising a linker, the linker covalently joins the first subunit and the second subunit. In some embodiments, the engineered meganuclease comprises an amino acid sequence having at least 80%, 85%, 90%, 91%, 92%, 93%, 94%, 95%, 96%, 97%, 98%, 99% or more sequence identity to SEQ ID NO: 4.

In some embodiments, the engineered meganuclease comprises an amino acid sequence of SEQ ID NO: 4. In some embodiments, the engineered meganuclease is encoded by a nucleotide sequence having at least 80%, 85%, 90%, 91%, 92%, 93%, 94%, 95%, 96%, 97%, 98%, 99% or more sequence identity to a nucleic acid sequence of SEQ ID NO: 5. In some embodiments, the engineered meganuclease is encoded by a nucleic acid sequence of SEQ ID NO: 5.

In some embodiments, the MTP comprises an amino acid sequence having at least 80%, 85%, 90%, 91%, 92%, 93%, 94%, 95%, 96%, 97%, 98%, 99% or more sequence identity to a sequence set forth in any one of SEQ ID NOs: 6-8. In some embodiments, the MTP comprises an amino acid sequence set forth in any one of SEQ ID NOs: 6-8. In some embodiments, the MTP is attached to the C-terminus of the engineered meganuclease. In some embodiments, the MTP is attached to the N-terminus of the engineered meganuclease. In some embodiments, the MTP is fused to the engineered meganuclease. In some embodiments, the MTP is attached to the engineered meganuclease by a polypeptide linker. In some embodiments, the engineered meganuclease is attached to a first MTP and a second MTP. In some embodiments, the first MTP and/or the second MTP comprises an amino acid sequence having at least 80%, 85%, 90%, 91%, 92%, 93%, 94%, 95%, 96%, 97%, 98%, 99% or more sequence identity to a sequence set forth in any one of SEQ ID NOs: 6-8. In some embodiments, the first MTP and/or the second MTP comprises an amino acid sequence set forth in any one of SEQ ID NOs: 6-8. In some embodiments, the first MTP and the second MTP are identical. In some embodiments, the first MTP and the second MTP are not identical. In some embodiments, the first MTP and/or the second MTP is fused to the engineered meganuclease. In some embodiments, the first MTP and/or the second MTP is attached to the engineered meganuclease by a polypeptide linker.

In some embodiments, the MTEM is attached to a nuclear export sequence (NES). In some embodiments, the NES comprises an amino acid sequence having at least 80%, 85%, 90%, 91%, 92%, 93%, 94%, 95%, 96%, 97%, 98%, 99% or more sequence identity to a sequence set forth in SEQ ID NO: 9 or 10. In some embodiments, the NES comprises an amino acid sequence set forth in SEQ ID NO: 9 or 10. In some embodiments, the NES is attached at the N-terminus of the MTEM. In some embodiments, the NES is attached at the C-terminus of the MTEM. In some embodiments, the NES is fused to the MTEM. In some embodiments, the NES is attached to the MTEM by a polypeptide linker. In some embodiments, the MTEM comprises a first NES and a second NES. In some embodiments, the first NES is attached at the N-terminus of the MTEM, and the second NES is attached at the C-terminus of the MTEM. In some embodiments, the first NES and/or the second NES comprises an amino acid sequence having at least 80%, 85%, 90%, 91%, 92%, 93%, 94%, 95%, 96%, 97%, 98%, 99% or more sequence identity to a sequence set forth in SEQ ID NO: 9 or 10. In some embodiments, the first NES and/or the second NES comprises an amino acid sequence set forth in SEQ ID NO: 9 or 10. In some embodiments, the first NES and the second NES are identical. In some embodiments, the first NES and the second NES are not identical. In some embodiments, the first NES and/or the second NES is fused to the MTEM. In some embodiments, the first NES and/or the second NES is attached to the MTEM. In some embodiments, the first NES and/or the second NES is attached to the MTEM.

In another aspect, the invention provides a polynucleotide comprising a nucleic acid sequence encoding an MTEM described herein. In some embodiments, the polynucleotide is an mRNA.

In another aspect, the invention provides a recombinant DNA construct comprising a polynucleotide comprising a nucleic acid sequence encoding an MTEM described herein. In some embodiments, the recombinant DNA construct encodes a recombinant virus comprising the polynucleotide. In some embodiments, the recombinant virus is a recombinant adenovirus, a recombinant lentivirus, a recombinant retrovirus, or a recombinant adeno-associated virus (AAV). In some embodiments, the recombinant virus is a recombinant AAV. In some embodiments, the recombinant AAV has an AAV2, AAV9, or AAVHSC capsid. In some embodiments, the polynucleotide comprises a promoter operably linked to the nucleic acid sequence encoding the MTEM. In some embodiments, the promoter is a constitutive promoter or a tissue-specific promoter. In some embodiments, the constitutive promoter is a CMV promoter, a CAG promoter, an EF1 alpha promoter, or a UbC promoter, or the tissue-specific promoter is a neuron-specific promoter, an astrocyte-specific promoter, a microglia-specific promoter, a muscle-specific promoter, a skeletal muscle-specific promoter, a myotube-specific promoter, muscle satellite cell-specific promoter, a cardiomyocyte-specific promoter, an eye-specific promoter, a retina-specific promoter, a retinal ganglion cell-specific promoter, a retinal pigmentary epithelium-specific promoter, a leukocyte-specific promoter, a progenitor cell-specific promoter, a blood progenitor cell-specific promoter, a pancreas-specific promoter, a pancreatic beta cell-specific promoter, an endothelial cell-specific promoter, an inner ear hair cell-specific promoter, a bone marrow cell-specific promoter, or a kidney-specific promoter.

In another aspect, the invention provides a plasmid comprising a recombinant DNA described herein.

In another aspect, the invention provides a recombinant virus comprising a polynucleotide comprising a nucleic acid sequence encoding an MTEM described herein. In some embodiments, the recombinant virus is a recombinant adenovirus, a recombinant lentivirus, a recombinant retrovirus, or a recombinant adeno-associated virus (AAV). In some embodiments, the recombinant virus is a recombinant AAV. In some embodiments, the recombinant AAV has an AAV2, AAV9, or AAVHSC capsid. In some embodiments, the polynucleotide comprises a promoter operably linked to the nucleic acid sequence encoding the MTEM. In some embodiments, the promoter is a constitutive promoter or a tissue-specific promoter. In some embodiments, the constitutive promoter is a CMV promoter, a CAG promoter, an EF1 alpha promoter, or a UbC promoter, or the tissue-specific promoter is a neuron-specific promoter, an astrocyte-specific promoter, a microglia-specific promoter, a muscle-specific promoter, a skeletal muscle-specific promoter, a myotube-specific promoter, muscle satellite cell-specific promoter, a cardiomyocyte-specific promoter, an eye-specific promoter, a retina-specific promoter, a retinal ganglion cell-specific promoter, a retinal pigmentary epithelium-specific promoter, a leukocyte-specific promoter, a progenitor cell-specific promoter, a blood progenitor cell-specific promoter, a pancreas-specific promoter, a pancreatic beta cell-specific promoter, an endothelial cell-specific promoter, an inner ear hair cell-specific promoter, a bone marrow cell-specific promoter, or a kidney-specific promoter.

In another aspect, the invention provides a lipid nanoparticle composition comprising lipid nanoparticles comprising a polynucleotide, wherein the polynucleotide comprises a nucleic acid sequence encoding an MTEM described herein. In some embodiments, the polynucleotide is an mRNA.

In another aspect, the invention provides a pharmaceutical composition comprising a pharmaceutically acceptable carrier and an MTEM described herein.

In another aspect, the invention provides a pharmaceutical composition comprising a pharmaceutically acceptable carrier and any polynucleotide described herein.

In another aspect, the invention provides a pharmaceutical composition comprising a pharmaceutically acceptable carrier and any recombinant DNA construct described herein.

In another aspect, the invention provides a pharmaceutical composition comprising a pharmaceutically acceptable carrier and any recombinant virus described herein.

In another aspect, the invention provides a pharmaceutical composition comprising a pharmaceutically acceptable carrier and any lipid nanoparticle composition described herein.

In another aspect, the invention provides a genetically-modified eukaryotic cell comprising any polynucleotide described herein. In some embodiments, the genetically-modified eukaryotic cell is a genetically-modified mammalian cell. In some embodiments, the genetically-modified eukaryotic cell is a genetically-modified human cell. In some embodiments, the genetically-modified eukaryotic cell is a genetically-modified plant cell.

In another aspect, the invention provides a method for producing a genetically-modified eukaryotic cell, the method comprising introducing into a eukaryotic cell: (a) a polynucleotide comprising a nucleic acid sequence encoding an MTEM described herein, wherein the MTEM is expressed in the eukaryotic cell; or (b) an MTEM described herein; wherein the MTEM produces a cleavage site at the recognition sequence in mitochondrial genomes of the eukaryotic cell. In some embodiments, the cleavage site is repaired by non-homologous end joining, such that the recognition sequence comprises an insertion or deletion. In some embodiments, the mitochondrial genomes comprising the recognition sequence are degraded in the genetically-modified eukaryotic cell. In some embodiments, the mitochondrial genomes are mutant mitochondrial genomes. In some embodiments, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99% of mutant mitochondrial genomes comprising the recognition sequence are degraded in the genetically-modified eukaryotic cell. In some embodiments, the ratio of wild-type mitochondrial genomes to mutant mitochondrial genomes comprising the recognition sequence increases in the genetically-modified eukaryotic cell. In some embodiments, the ratio increases to about 5:95, about 10:90, about 15:85, about 20:80, about 25:75, about 30:70, about 35:65, about 40:60, about 45:55, about 50:50, about 55:45, about 60:40, about 65:35, about 70:30, about 75:25, about 80:20, about 85:15, about 90:10, about 95:5, about 20:1, about 50:1, about 100:1, about 150:1, about 200:1, about 250:1, about 300:1, about 350:1, about 400:1, about 450:1, about 500:1, about 550:1, about 600:1, about 650:1, about 700:1, about 750:1, about 800:1, about 850:1, about 900:1, about 950:1, about 1000:1, or more. In some embodiments, the percentage of wild-type mitochondrial genomes in the genetically-modified eukaryotic cell is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more, of the total mitochondrial genomes in the genetically-modified eukaryotic cell. In some embodiments, the percentage of mutant mitochondrial genomes comprising the recognition sequence in the genetically-modified eukaryotic cell decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more. In some embodiments, cellular respiration in the genetically-modified eukaryotic cell increases by about 30%, about 35%, about 40%, about 45%, about 50%, about 55%, about 60%, about 65%, about 70%, about 75%, about 80%, about 85%, about 90%, about 95%, about 100%, or more. In some embodiments, cellular respiration in the genetically-modified eukaryotic cell increases by about 30-40%, about 40-50%, about 50-60%, about 60-70%, about 70-80%, about 80-90%, about 90-100%, or more.

In another aspect, the invention provides a method for producing a population of eukaryotic cells comprising a plurality of genetically-modified cells, the method comprising introducing into a plurality of eukaryotic cells in the population: (a) a polynucleotide comprising a nucleic acid sequence encoding an MTEM described herein, wherein the MTEM is expressed in the plurality of eukaryotic cells; or (b) an MTEM described herein; wherein the MTEM produces a cleavage site at the recognition sequence in mitochondrial genomes of the plurality of eukaryotic cells. In some embodiments, the cleavage site is repaired by non-homologous end joining, such that the recognition sequence comprises an insertion or deletion. In some embodiments, the mitochondrial genomes comprising the recognition sequence are degraded in the plurality of genetically-modified eukaryotic cells. In some embodiments, the mitochondrial genomes are mutant mitochondrial genomes. In some embodiments, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99% of mutant mitochondrial genomes comprising the recognition sequence are degraded in the plurality of genetically-modified eukaryotic cells. In some embodiments, the ratio of wild-type mitochondrial genomes to mutant mitochondrial genomes comprising the recognition sequence increases in the plurality of genetically-modified eukaryotic cells. In some embodiments, the ratio of wild-type mitochondrial genomes to mutant mitochondrial genomes comprising the recognition sequence increases in the population of eukaryotic cells. In some embodiments, the ratio increases to about 5:95, about 10:90, about 15:85, about 20:80, about 25:75, about 30:70, about 35:65, about 40:60, about 45:55, about 50:50, about 55:45, about 60:40, about 65:35, about 70:30, about 75:25, about 80:20, about 85:15, about 90:10, about 95:5, about 20:1, about 50:1, about 100:1, about 150:1, about 200:1, about 250:1, about 300:1, about 350:1, about 400:1, about 450:1, about 500:1, about 550:1, about 600:1, about 650:1, about 700:1, about 750:1, about 800:1, about 850:1, about 900:1, about 950:1, about 1000:1, or more. In some embodiments, the percentage of wild-type mitochondrial genomes in the plurality of genetically-modified eukaryotic cells increases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more. In some embodiments, the percentage of wild-type mitochondrial genomes in the population of eukaryotic cells increases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more. In some embodiments, the percentage of mutant mitochondrial genomes comprising the recognition sequence in the plurality of genetically-modified eukaryotic cells decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more. In some embodiments, the percentage of mutant mitochondrial genomes comprising the recognition sequence in the population of eukaryotic cells decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more. In some embodiments, cellular respiration in the plurality of genetically-modified eukaryotic cells increases by about 30%, about 35%, about 40%, about 45%, about 50%, about 55%, about 60%, about 65%, about 70%, about 75%, about 80%, about 85%, about 90%, about 95%, about 100%, or more. In some embodiments, cellular respiration in the plurality of genetically-modified eukaryotic cells increases by about 30-40%, about 40-50%, about 50-60%, about 60-70%, about 70-80%, about 80-90%, about 90-100%, or more. In some embodiments, cellular respiration in the population of eukaryotic cells increases by about 30%, about 35%, about 40%, about 45%, about 50%, about 55%, about 60%, about 65%, about 70%, about 75%, about 80%, about 85%, about 90%, about 95%, about 100%, or more. In some embodiments, cellular respiration in the population of eukaryotic cells increases by about 30-40%, about 40-50%, about 50-60%, about 60-70%, about 70-80%, about 80-90%, about 90-100%, or more.

In some embodiments, the method is performed in vivo. In some embodiments, the method is performed in vitro. In some embodiments, the polynucleotide is an mRNA. In some embodiments, the polynucleotide is any mRNA described herein. In some embodiments, the polynucleotide is a recombinant DNA construct. In some embodiments, the polynucleotide is any recombinant DNA construct described herein. In some embodiments, the polynucleotide is introduced into the eukaryotic cell by a lipid nanoparticle. In some embodiments, the polynucleotide is introduced into the eukaryotic cell by a recombinant virus. In some embodiments, the recombinant virus is any recombinant virus described herein. In some embodiments, the recombinant virus is a recombinant AAV. In some embodiments, the recombinant AAV has an AAV2, AAV9, or AAVHSC capsid. In some embodiments, the polynucleotide comprises a promoter operably linked to the nucleic acid sequence encoding the MTEM. In some embodiments, the promoter is a constitutive promoter or a tissue-specific promoter. In some embodiments, the constitutive promoter is a CMV promoter, a CAG promoter, an EF1 alpha promoter, or a UbC promoter, or the tissue-specific promoter is a neuron-specific promoter, an astrocyte-specific promoter, a microglia-specific promoter, a muscle-specific promoter, a skeletal muscle-specific promoter, a myotube-specific promoter, muscle satellite cell-specific promoter, a cardiomyocyte-specific promoter, an eye-specific promoter, a retina-specific promoter, a retinal ganglion cell-specific promoter, a retinal pigmentary epithelium-specific promoter, a leukocyte-specific promoter, a progenitor cell-specific promoter, a blood progenitor cell-specific promoter, a pancreas-specific promoter, a pancreatic beta cell-specific promoter, an endothelial cell-specific promoter, an inner ear hair cell-specific promoter, a bone marrow cell-specific promoter, or a kidney-specific promoter. In some embodiments, the eukaryotic cell is a mammalian cell. In some embodiments, the eukaryotic cell is a human cell. In some embodiments, the eukaryotic cell is a neuron, an astrocyte, a microglia cell, a muscle cell, a skeletal muscle cell, a myotube cell, a muscle satellite cell, a cardiomyocyte, a cell of the eye, a retinal cell, a retinal ganglion cell, a retinal pigmentary epithelium cell, a leukocyte, a progenitor cell, a blood progenitor cell, a pancreas cell, a pancreatic beta cell, an endothelial cell, an inner ear hair cell, a bone marrow cell, or a kidney cell. In some embodiments, the eukaryotic cell is a plant cell.

In another aspect, the invention provides a genetically-modified eukaryotic cell, or a population of genetically-modified eukaryotic cells, produced by any method for producing a genetically-modified eukaryotic cell or any method for producing a population of eukaryotic cells comprising a plurality of genetically-modified cells.

In another aspect, the invention provides a method for degrading mutant mitochondrial genomes in a target cell in a subject, or in a population of target cells in a subject, the method comprising delivering to the target cell or the population of target cells: (a) a polynucleotide comprising a nucleic acid sequence encoding an MTEM described herein, wherein the MTEM is expressed in the target cell or the population of target cells; or (b) an MTEM described herein; wherein the MTEM produces a cleavage site in the mutant mitochondrial genomes at a recognition sequence, and wherein the mutant mitochondrial genomes are degraded. In some embodiments, the subject is a mammal. In some embodiments, the subject is a human. In some embodiments, the target cell is a neuron, an astrocyte, a microglia cell, a muscle cell, a skeletal muscle cell, a myotube cell, a muscle satellite cell, a cardiomyocyte, a cell of the eye, a retinal cell, a retinal ganglion cell, a retinal pigmentary epithelium cell, a leukocyte, a progenitor cell, a blood progenitor cell, a pancreas cell, a pancreatic beta cell, an endothelial cell, an inner ear hair cell, or a kidney cell, or the population of target cells is a population of neurons, astrocytes, microglia cells, muscle cells, skeletal muscle cells, myotube cells, muscle satellite cells, cardiomyocytes, cells of the eye, retinal cells, retinal ganglion cells, retinal pigmentary epithelium cells, leukocytes, progenitor cells, blood progenitor cells, pancreas cells, pancreatic beta cells, endothelial cells, inner ear hair cells, or kidney cells. In some embodiments, the polynucleotide is an mRNA. In some embodiments, the polynucleotide is any mRNA described herein. In some embodiments, the polynucleotide is a recombinant DNA construct. In some embodiments, the polynucleotide is any recombinant DNA construct described herein. In some embodiments, the polynucleotide is delivered to the target cell, or the population of target cells, by a lipid nanoparticle. In some embodiments, the polynucleotide is delivered to the target cell, or the population of target cells, by a recombinant virus. In some embodiments, the recombinant virus is any recombinant virus described herein. In some embodiments, the recombinant virus is a recombinant AAV. In some embodiments, the recombinant AAV has an AAV2, AAV9, or AAVHSC capsid. In some embodiments, the polynucleotide comprises a promoter operably linked to the nucleic acid sequence encoding the MTEM. In some embodiments, the promoter is a constitutive promoter or a tissue-specific promoter. In some embodiments, the constitutive promoter is a CMV promoter, a CAG promoter, an EF1 alpha promoter, or a UbC promoter, or the tissue-specific promoter is a neuron-specific promoter, an astrocyte-specific promoter, a microglia-specific promoter, a muscle-specific promoter, a skeletal muscle-specific promoter, a myotube-specific promoter, muscle satellite cell-specific promoter, a cardiomyocyte-specific promoter, an eye-specific promoter, a retina-specific promoter, a retinal ganglion cell-specific promoter, a retinal pigmentary epithelium-specific promoter, a leukocyte-specific promoter, a progenitor cell-specific promoter, a blood progenitor cell-specific promoter, a pancreas-specific promoter, a pancreatic beta cell-specific promoter, an endothelial cell-specific promoter, an inner ear hair cell-specific promoter, a bone marrow cell-specific promoter, or a kidney-specific promoter. In some embodiments, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99% of mutant mitochondrial genomes comprising the recognition sequence are degraded in the target cell or the population of the target cells. In some embodiments, the ratio of wild-type mitochondrial genomes to mutant mitochondrial genomes comprising the recognition sequence increases in the target cell or the population of target cells. In some embodiments, the ratio increases to about 5:95, about 10:90, about 15:85, about 20:80, about 25:75, about 30:70, about 35:65, about 40:60, about 45:55, about 50:50, about 55:45, about 60:40, about 65:35, about 70:30, about 75:25, about 80:20, about 85:15, about 90:10, about 95:5, about 20:1, about 50:1, about 100:1, about 150:1, about 200:1, about 250:1, about 300:1, about 350:1, about 400:1, about 450:1, about 500:1, about 550:1, about 600:1, about 650:1, about 700:1, about 750:1, about 800:1, about 850:1, about 900:1, about 950:1, about 1000:1, or more. In some embodiments, the percentage of wild-type mitochondrial genomes in the target cell or the population of target cells is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more, of the total mitochondrial genomes in the target cell or the population of target cells. In some embodiments, the percentage of mutant mitochondrial genomes comprising the recognition sequence in the genetically-modified eukaryotic cell decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more. In some embodiments, cellular respiration in the target cell or the population of target cells increases by about 30%, about 35%, about 40%, about 45%, about 50%, about 55%, about 60%, about 65%, about 70%, about 75%, about 80%, about 85%, about 90%, about 95%, about 100%, or more. In some embodiments, cellular respiration in the target cell or the population of target cells increases by about 30-40%, about 40-50%, about 50-60%, about 60-70%, about 70-80%, about 80-90%, about 90-100%, or more.

In another aspect, the invention provides a method for treating a condition associated with a mitochondrial disorder in a subject, the method comprising administering to the subject: (a) a therapeutically-effective amount of a polynucleotide comprising a nucleic acid sequence encoding an MTEM described herein, wherein the polynucleotide is delivered to a target cell, or a population of target cells, in the subject, and wherein the MTEM is expressed in the target cell or the population of target cells; or (b) a therapeutically-effective amount of an MTEM described herein, wherein the MTEM is delivered to a target cell, or a population of target cells, in the subject; wherein the MTEM produces a cleavage site in mutant mitochondrial genomes at a recognition sequence, and wherein the mutant mitochondrial genomes are degraded. In some embodiments, the method comprises administering any pharmaceutical composition described herein. In some embodiments, the subject is a mammal. In some embodiments, the subject is a human. In some embodiments, the target cell is a neuron, an astrocyte, a microglia cell, a muscle cell, a skeletal muscle cell, a myotube cell, a muscle satellite cell, a cardiomyocyte, a cell of the eye, a retinal cell, a retinal ganglion cell, a retinal pigmentary epithelium cell, a leukocyte, a progenitor cell, a blood progenitor cell, a pancreas cell, a pancreatic beta cell, an endothelial cell, an inner ear hair cell, or a kidney cell, or the population of target cells is a population of neurons, astrocytes, microglia cells, muscle cells, skeletal muscle cells, myotube cells, muscle satellite cells, cardiomyocytes, cells of the eye, retinal cells, retinal ganglion cells, retinal pigmentary epithelium cells, leukocytes, progenitor cells, blood progenitor cells, pancreas cells, pancreatic beta cells, endothelial cells, inner ear hair cells, or kidney cells. In some embodiments, the condition is a condition of the muscle, heart, central nervous system, eye, bone marrow, kidney, pancreas, white blood cells, blood vessels, or inner ear. In some embodiments, the condition is Pearson Syndrome, Progressive external Ophthalmoplegia, Kearns-Sayre Syndrome (KSS), Myoclonic Epilepsy with Ragged Red Fibers (MERRF), Neuropathy, Ataxia, Retinitis Pigmentosa (NARP), Leber Hereditary Optic Neuropathy (LHON), Chronic Progressive External Ophthalmoplegia (CPEO), Maternally Inherited Leigh Syndrome (MILS), Maternally Inherited Diabetes and Deafness (MIDD), or mitochondria disorders with overlap symptoms. In some embodiments, the polynucleotide is an mRNA. In some embodiments, the polynucleotide is any mRNA described herein. In some embodiments, the polynucleotide is a recombinant DNA construct. In some embodiments, the polynucleotide is any recombinant DNA construct described herein. In some embodiments, the polynucleotide is delivered to the target cell, or the population of target cells, by a lipid nanoparticle. In some embodiments, the polynucleotide is delivered to the target cell, or the population of target cells, by a recombinant virus. In some embodiments, the recombinant virus is any recombinant virus described herein. In some embodiments, the recombinant virus is a recombinant AAV. In some embodiments, the recombinant AAV has an AAV2, AAV9, or AAVHSC capsid. In some embodiments, the polynucleotide comprises a promoter operably linked to the nucleic acid sequence encoding the MTEM. In some embodiments, the promoter is a constitutive promoter or a tissue-specific promoter. In some embodiments, the constitutive promoter is a CMV promoter, a CAG promoter, an EF1 alpha promoter, or a UbC promoter, or the tissue-specific promoter is a neuron-specific promoter, an astrocyte-specific promoter, a microglia-specific promoter, a muscle-specific promoter, a skeletal muscle-specific promoter, a myotube-specific promoter, muscle satellite cell-specific promoter, a cardiomyocyte-specific promoter, an eye-specific promoter, a retina-specific promoter, a retinal ganglion cell-specific promoter, a retinal pigmentary epithelium-specific promoter, a leukocyte-specific promoter, a progenitor cell-specific promoter, a blood progenitor cell-specific promoter, a pancreas-specific promoter, a pancreatic beta cell-specific promoter, an endothelial cell-specific promoter, an inner ear hair cell-specific promoter, a bone marrow cell-specific promoter, or a kidney-specific promoter. In some embodiments, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99% of mutant mitochondrial genomes comprising the recognition sequence are degraded in the target cell or the population of the target cells. In some embodiments, the ratio of wild-type mitochondrial genomes to mutant mitochondrial genomes comprising the recognition sequence increases in the target cell or the population of target cells. In some embodiments, the ratio increases to about 5:95, about 10:90, about 15:85, about 20:80, about 25:75, about 30:70, about 35:65, about 40:60, about 45:55, about 50:50, about 55:45, about 60:40, about 65:35, about 70:30, about 75:25, about 80:20, about 85:15, about 90:10, about 95:5, about 20:1, about 50:1, about 100:1, about 150:1, about 200:1, about 250:1, about 300:1, about 350:1, about 400:1, about 450:1, about 500:1, about 550:1, about 600:1, about 650:1, about 700:1, about 750:1, about 800:1, about 850:1, about 900:1, about 950:1, about 1000:1, or more. In some embodiments, the percentage of wild-type mitochondrial genomes in the target cell or the population of target cells is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more, of the total mitochondrial genomes in the target cell or the population of target cells. In some embodiments, the percentage of mutant mitochondrial genomes comprising the recognition sequence in the genetically-modified eukaryotic cell decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more. In some embodiments, cellular respiration in the target cell or the population of target cells increases by about 30%, about 35%, about 40%, about 45%, about 50%, about 55%, about 60%, about 65%, about 70%, about 75%, about 80%, about 85%, about 90%, about 95%, about 100%, or more. In some embodiments, cellular respiration in the target cell or the population of target cells increases by about 30-40%, about 40-50%, about 50-60%, about 60-70%, about 70-80%, about 80-90%, about 90-100%, or more.

### BRIEF DESCRIPTION OF THE FIGURES

FIG. 1 shows the MTEM construct and mitochondrial expression. FIG. 1A describes the strategy for mitochondrial cleavage detection. In order to test recognition sequence cleavage of an engineered nuclease (against the mouse mtDNA tRNA-Ala mutation [MIT 11-12]), a pair of engineered CHO lines were produced to carry either the wild-type (WT) or mutant mtDNA target site in the nuclear DNA. The target site was positioned between direct repeats of a GFP gene such that cleavage of the target site promotes homologous recombination events between repeated regions to yield a functional GFP. Additionally, there is a target site for a positive control nuclease (“CHO 23-24”) incorporated next to the engineered nuclease target site (left panel). Each of the cell lines was transfected with mRNA encoding MIT 11-12, or CHO 23-24 (control) and cells were assayed by flow cytometry 48 hours post-transfection for the percentage of GFP+ cells (right panel). FIG. 1B depicts the MTEM gene construct for ex vivo expression including a CMV promoter, mitochondrial transit peptide (MTP) of Cox8 or Cox8/Su9, Flag tag for immunological detection, engineered meganuclease (MTEM) sequence, and PolyA tail. FIG. 1C shows immunofluorescence done on HeLa cells 24 hours after transfection with MTEM. MitoTracker stains mitochondria red, Flag stains MTEM green, and merged image shows co-localization (yellow) of MTEM to mitochondria. Images taken at 40× magnification. FIG. 1D reports Western blot results depicting MTEM expression (FLAG) in HEK293T cells 24 hours after transfection with either CF or CSF construct. Lanes CF+GFP and CSF+GFP depict protein expression in cells transfected with MTEM constructs in which a GFP sequence was added. Lane Unt represents untransfected cells. Lane GFP represents cells transfected with GFP only. Tubulin (Tub) expression was used as a loading control.

FIG. 2 shows the effect of MTEM on heteroplasmic cells carrying the tRNA^{Ala} mutation (m.5024C>T). FIG. 2A reports examples of FACS cell sorting gating. Cells were sorted by the presence of GFP co-expression: “Black” cells (bottom gate) and “Green” cells (top gate). FIG. 2B reports RFLP-HOT PCR analysis of two independent transfections and cell sorting experiments of heteroplasmic cells carrying 50% m.5024C>T mutation. Mutant levels in the Green cell populations (Gr) were compared to Untransfected cells (U). FIG. 2C shows quantification of heteroplasmy shift from the two cell sorting experiments in cells carrying 50% mutation described in FIG. 2B. Results were compared to Untransfected cell heteroplasmy. FIG. 2D shows RFLP “last cycle hot” PCR analysis of heteroplasmic cells carrying high heteroplasmic mutant load (90%) transfected with MTEM over time. FIG. 2E shows quantification of results in FIG. 2D. Values are normalized to untransfected cells. Black cells are named Blk (n=4). FIG. 2F reports the total mtDNA levels checked in highly mutant cells transfected with MTEM, and compared to untransfected cells 24 hours after transfection, and followed for three weeks after transfection (n=3). FIG. 2G shows the Oxygen Consumption Rate (OCR) deduced in cells carrying high levels of heteroplasmic mutant mtDNA that were transfected with MTEM and grown for three weeks (n=3-7). Data are mean±SEM. Statistical analysis was performed using two-tailed student's t-test. p≤0.05*, p≤0.01**, p>0.001

FIG. 3 reports the effect of AAV9-MTEM in treated juvenile mice. FIG. 3A depicts representative Western blots (WB) of homogenates (top panels) with Flag antibody for AAV9-MTEM samples and GFP antibody for AAV-GFP samples. RFLP “last cycle hot” PCR analysis (RFLP, bottom panels) of DNA samples from the same injected animals at 6, 12, and 24 weeks PI. FIG. 3B shows the quantification of heteroplasmy shift shown as a percent change in heteroplasmy across all tissues at 6, 12, and 24 weeks PI normalized to brain tissue. Heteroplasmy levels of heart (H), tibialis anterior (TA), quadriceps (Q), gastrocnemius (G), kidney (K), liver (L), and spleen (Sp) were compared to brain (B) (negative for expression of MTEM). FIG. 3C shows the quantification by RT-PCR of total mtDNA levels in skeletal muscle, liver, and brain at 6 and 24 weeks PI using ND1 and ND5 mitochondrial primer/probes normalized to 18S (nuclear DNA). Data are mean±SEM of n=3-5. Statistical analysis was performed using two-tailed student's t-test. p≤0.05*, p≤0.01**, p≤0.001***, p≤0.0001****

FIG. 4 shows the effected of AAV9-MTEM in treated adult mice. FIG. 4A depicts representative western blots (W.B.) of homogenates (top panels) with Flag antibody for AAV9-MTEM samples and GFP antibody for AAV-GFP samples. RFLP “last cycle hot” PCR analysis (RFLP, bottom panels) of DNA samples from the same injected animals, at 6, 12, and 24 weeks PI. FIG. 4B shows the quantification of heteroplasmy shift shown as percent change in heteroplasmy across all tissues at 6, 12, and 24 weeks PI normalized to brain tissue. Heteroplasmy of heart (H), tibialis anterior (TA), quadriceps (Q), gastrocnemius (G), kidney (K), liver (L), and spleen (Sp) were compared to brain (negative for expression of MTEM). FIG. 4C shows the quantification by qPCR of total mtDNA levels were measured in skeletal muscle, liver, and brain (B) at 6- and 24-weeks PI using ND1 and ND5 mitochondrial primer/probes normalized to 18S (nuclear DNA). Data are mean±SEM of n=3-4. Statistical analysis was performed using two-tailed student's t-test. p≤0.05*, p≤0.01**, p≤0.001*** p≤0.0001****

FIG. 5 reports an MTEM-induced increase in mt-tRNA^{Ala} in liver. FIG. 5A shows a northern blot analysis of juvenile mouse liver 24 weeks PI probed for mt-tRNA^{Ala} and total RNA loading (28S and 18S). FIG. 5B shows the quantification of mt-tRNA^{Ala} in Northern blot in FIG. 5A normalized to 28S rRNA. FIG. 5C shows the quantification of mt-tRNA^{Ala} by qPCR compared to levels of mt-tRNA Val in juvenile mouse liver. RNA samples from AAV9-MTEM treated animals were compared to AAV9-GFP controls and WT liver samples. FIG. 5D reports quantification of mt-tRNA^{Ala} compared to levels of mt-tRNA^{Val} in adult mouse liver. RNA samples from AAV9-MTEM treated animals compared to AAV9-GFP controls and WT liver samples, at 24 weeks PI. Data are mean±SEM of n=3-4. Statistical analysis was performed using two-tailed student's t-test. p<0.05*

FIG. 6 shows indel formation by an engineered meganuclease fused to a nuclear localization signal (NLS), a mitochondrial transit peptide (MTP), or an MTP and the nuclear export sequence (NES) of SEQ ID NO: 10. MRC-5 cells were nucleofected with an engineered meganuclease construct and indel formation at the APC 11-12 binding site was analyzed 2 days later.

FIG. 7 shows indel formation by an engineered meganuclease fused to a nuclear localization signal (NLS), a mitochondrial transit peptide (MTP), or an MTP and the MVMp NS2 nuclear export sequence (NES) of SEQ ID NO: 9. MRC-5 cells were nucleofected with an engineered meganuclease construct and indel formation at the APC 11-12 binding site was analyzed 2 days later.

### BRIEF DESCRIPTION OF THE SEQUENCES

SEQ ID NO: 1 sets forth the Wild-type I-CreI sequence.

SEQ ID NO: 2 sets forth the MTEM meganuclease with wild-type I-CreI subunits.

SEQ ID NO: 3 sets forth the MIT 11-12 recognition sequence (sense).

SEQ ID NO: 4 sets forth the MIT 11-12x.40 meganuclease amino acid sequence.

SEQ ID NO: 5 sets forth the MIT 11-12x.40 meganuclease nucleic acid sequence.

SEQ ID NO: 6 sets forth the COX VIII MTP.

SEQ ID NO: 7 sets forth the SU9 MTP.

SEQ ID NO: 8 sets forth the COX VIII-SU9 MTP.

SEQ ID NO: 9 sets forth the MVMp NS2 NES sequence.

SEQ ID NO: 10 sets forth the NES sequence.

SEQ ID NO: 11 sets forth the MIT 11-12 recognition sequence (antisense).

SEQ ID NO: 12 sets forth the m.5024C>T forward primer.

SEQ ID NO: 13 sets forth the m.5024C>T reverse primer.

SEQ ID NO: 14 sets forth the ND1 forward primer.

SEQ ID NO: 15 sets forth the ND1 reverse primer.

SEQ ID NO: 16 sets forth the ND1 probe.

SEQ ID NO: 17 sets forth the ND5 forward primer.

SEQ ID NO: 18 sets forth the ND5 reverse primer.

SEQ ID NO: 19 sets forth the ND5 probe.

SEQ ID NO: 20 sets forth the 18s forward primer.

SEQ ID NO: 21 sets forth the 18s reverse primer.

SEQ ID NO: 22 sets forth the 18s probe.

SEQ ID NO: 23 sets forth the biotinylated probe to detect mitochondrial tRNA^{ala}.

SEQ ID NO: 24 sets forth the C57BL6J chromosome 2 site 1.

SEQ ID NO: 25 sets forth the C57BL6J chromosome 2 site 2.

SEQ ID NO: 26 sets forth the C57BL6J chromosome 5 site 3.

SEQ ID NO: 27 sets forth the C57BL6J chromosome 6 site 4.

SEQ ID NO: 28 sets forth the C57BL6J chromosome 2 site 5.

SEQ ID NO: 29 sets forth the probe for APC 11-12 binding site.

SEQ ID NO: 30 sets forth the forward primer for APC 11-12 binding site.

SEQ ID NO: 31 sets forth the reverse primer for APC 11-12 binding site.

SEQ ID NO: 32 sets forth the probe for reference binding site.

SEQ ID NO: 33 sets forth the forward primer for reference binding site.

SEQ ID NO: 34 sets forth the reverse primer for reference binding site.

### DETAILED DESCRIPTION OF THE INVENTION

### 1.1 References and Definitions

The patent and scientific literature referred to herein establishes knowledge that is available to those of skill in the art. The issued US patents, allowed applications, published foreign applications, and references, including GenBank database sequences, which are cited herein are hereby incorporated by reference to the same extent as if each was specifically and individually indicated to be incorporated by reference.

The present invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference herein in their entirety.

As used herein, “a,” “an,” or “the” can mean one or more than one. For example, “a” cell can mean a single cell or a multiplicity of cells.

As used herein, the term “5′ cap” (also termed an RNA cap, an RNA 7-methylguanosine cap or an RNA m7G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from RNases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

As used herein, the term “allele” refers to one of two or more variant forms of a gene.

As used herein, the term “constitutive promoter” refers to a nucleotide sequence which, when operably linked with a polynucleotide which encodes or specifies a gene product, causes the gene product to be produced in a cell under most or all physiological conditions of the cell.

As used herein, the term “a control” or “a control cell” refers to a cell that provides a reference point for measuring changes in genotype or phenotype of a genetically-modified cell. A control cell may comprise, for example: (a) a wild-type cell, i.e., of the same genotype as the starting material for the genetic alteration which resulted in the genetically-modified cell; (b) a cell of the same genotype as the genetically-modified cell but which has been transformed with a null construct (i.e., with a construct which has no known effect on the trait of interest); or, (c) a cell genetically identical to the genetically-modified cell but which is not exposed to conditions or stimuli or further genetic modifications that would induce expression of altered genotype or phenotype. For example, a control or control cell of the instant invention can be a cell or population of cells that does not comprise an MTEM or a polynucleotide having an amino acid sequence encoding an MTEM.

As used herein, the term “corresponding to” with respect to modifications of two proteins or amino acid sequences is used to indicate that a specified modification in the first protein is a substitution of the same amino acid residue as in the modification in the second protein, and that the amino acid position of the modification in the first protein corresponds to or aligns with the amino acid position of the modification in the second protein when the two proteins are subjected to standard sequence alignments (e.g., using the BLASTp program). Thus, the modification of residue “X” to amino acid “A” in the first protein will correspond to the modification of residue “Y” to amino acid “A” in the second protein if residues X and Y correspond to each other in a sequence alignment and despite the fact that X and Y may be different numbers.

As used herein, the term “disrupted” or “disrupts” or “disrupts expression” or “disrupting a target sequence” refers to the introduction of a mutation (e.g., frameshift mutation) that interferes with the gene function and prevents expression and/or function of the polypeptide/expression product encoded thereby. For example, nuclease-mediated disruption of a gene can result in the expression of a truncated protein and/or expression of a protein that does not retain its wild-type function. Additionally, introduction of a donor template into a gene can result in no expression of an encoded protein, expression of a truncated protein, and/or expression of a protein that does not retain its wild-type function.

As used herein, the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “endogenous” in reference to a nucleotide sequence or protein is intended to mean a sequence or protein that is naturally comprised within or expressed by a cell.

As used herein, the terms “exogenous” or “heterologous” in reference to a nucleotide sequence or amino acid sequence are intended to mean a sequence that is purely synthetic, that originates from a foreign species, or, if from the same species, is substantially modified from its native form in composition and/or genomic locus by deliberate human intervention.

As used herein, the term “expression” refers to the transcription and/or translation of a particular nucleotide sequence driven by a promoter.

As used herein, the term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

As used herein, the term “genetically-modified” refers to a cell or organism in which, or in an ancestor of which, a genomic DNA sequence has been deliberately modified by recombinant technology. As used herein, the term “genetically-modified” encompasses the term “transgenic.” For example, as used herein, a “genetically-modified” cell may refer to a cell wherein the mitochondrial DNA has been deliberately modified by recombinant technology.

As used herein, the term “homologous recombination” or “HR” refers to the natural, cellular process in which a double-stranded DNA-break is repaired using a homologous DNA sequence as the repair template (see, e.g., Cahill et al. (2006), Front. Biosci. 11:1958-1976). The homologous DNA sequence may be an endogenous chromosomal sequence or an exogenous nucleic acid that was delivered to the cell.

As used herein, the term “homology arms” or “sequences homologous to sequences flanking a nuclease cleavage site” refer to sequences flanking the 5′ and 3′ ends of a nucleic acid molecule, which promote insertion of the nucleic acid molecule into a cleavage site generated by a nuclease. In general, homology arms can have a length of at least 50 base pairs, preferably at least 100 base pairs, and up to 2000 base pairs or more, and can have at least 90%, preferably at least 95%, or more, sequence homology to their corresponding sequences in the genome. In some embodiments, the homology arms are about 500 base pairs.

As used herein, the term “in vitro transcribed RNA” refers to RNA, preferably mRNA, which has been synthesized in vitro. Generally, the in vitro transcribed RNA is generated from an in vitro transcription vector. The in vitro transcription vector comprises a template that is used to generate the in vitro transcribed RNA.

As used herein, the term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

As used herein, the term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

As used herein, the term “lipid nanoparticle” refers to a lipid composition having a typically spherical structure with an average diameter between 10 and 1000 nanometers. In some formulations, lipid nanoparticles can comprise at least one cationic lipid, at least one non-cationic lipid, and at least one conjugated lipid. Lipid nanoparticles known in the art that are suitable for encapsulating nucleic acids, such as mRNA, are contemplated for use in the invention.

As used herein, the term “modification” with respect to recombinant proteins means any insertion, deletion, or substitution of an amino acid residue in the recombinant sequence relative to a reference sequence (e.g., a wild-type or a native sequence).

As used herein, the term “non-homologous end-joining” or “NHEJ” refers to the natural, cellular process in which a double-stranded DNA-break is repaired by the direct joining of two non-homologous DNA segments (see, e.g. Cahill et al. (2006), Front. Biosci. 11:1958-1976). DNA repair by non-homologous end-joining is error-prone and frequently results in the untemplated addition or deletion of DNA sequences at the site of repair. In some instances, cleavage at a target recognition sequence results in NHEJ at a target recognition site. Nuclease-induced cleavage of a target site in the coding sequence of a gene followed by DNA repair by NHEJ can introduce mutations into the coding sequence, such as frameshift mutations, that disrupt gene function. Thus, engineered nucleases can be used to effectively knock-out a gene in a population of cells.

As used herein, the term “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain one or more introns.

As used herein, the term “operably linked” is intended to mean a functional linkage between two or more elements. For example, an operable linkage between a nucleic acid sequence encoding a nuclease as disclosed herein and a regulatory sequence (e.g., a promoter) is a functional link that allows for expression of the nucleic acid sequence encoding the nuclease. Operably linked elements may be contiguous or non-contiguous. When used to refer to the joining of two protein coding regions, by operably linked is intended that the coding regions are in the same reading frame.

As used herein, unless specifically indicated otherwise, the word “or” is used in the inclusive sense of “and/or” and not the exclusive sense of “either/or.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

As used herein, the term “reduced” or “decreased” refers to a reduction in the percentage of cells or ratio of cells in a population of cells that comprise mutant mitochondrial genomes when compared to a population of control cells. In some embodiments, “reduced” or “decreased” refers to a reduction in the percentage of mutant mitochondrial genomes or ratio of mutant mitochondrial genomes to wild-type mitochondrial genomes in a single cell or in a population of cells. Such a reduction is up to 5%, 10%, 20%, 30%, 40%, 50%, 60%, 70%, 80%, 90%, 95%, or up to 100%. Accordingly, the term “reduced” encompasses both a partial reduction and a complete reduction of mutant mtDNA.

As used herein, the term with respect to both amino acid sequences and nucleic acid sequences, the terms “percent identity,” “sequence identity,” “percentage similarity,” “sequence similarity” and the like refer to a measure of the degree of similarity of two sequences based upon an alignment of the sequences that maximizes similarity between aligned amino acid residues or nucleotides, and which is a function of the number of identical or similar residues or nucleotides, the number of total residues or nucleotides, and the presence and length of gaps in the sequence alignment. A variety of algorithms and computer programs are available for determining sequence similarity using standard parameters. As used herein, sequence similarity is measured using the BLASTp program for amino acid sequences and the BLASTn program for nucleic acid sequences, both of which are available through the National Center for Biotechnology Information (www.ncbi.nlm.nih.gov/), and are described in, for example, Altschul et al. (1990), J. Mol. Biol. 215:403-410; Gish and States (1993), Nature Genet. 3:266-272; Madden et al. (1996), Meth. Enzymol.266:131-141; Altschul et al. (1997), Nucleic Acids Res. 25:33 89-3402); Zhang et al. (2000), J. Comput. Biol. 7(1-2):203-14. As used herein, percent similarity of two amino acid sequences is the score based upon the following parameters for the BLASTp algorithm: word size=3; gap opening penalty=−11; gap extension penalty=−1; and scoring matrix=BLOSUM62. As used herein, percent similarity of two nucleic acid sequences is the score based upon the following parameters for the BLASTn algorithm: word size=11; gap opening penalty=−5; gap extension penalty=−2; match reward=1; and mismatch penalty=−3.

As used herein, the term “poly(A)” is a series of adenosines attached by polyadenylation to the mRNA. In the preferred embodiment of a construct for transient expression, the poly A is between 50 and 5000, preferably greater than 64, more preferably greater than 100, most preferably greater than 300 or 400. Poly(A) sequences can be modified chemically or enzymatically to modulate mRNA functionality such as localization, stability or efficiency of translation.

As used herein, the term “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, the term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

As used herein, the terms “recombinant” or “engineered,” with respect to a protein, means having an altered amino acid sequence as a result of the application of genetic engineering techniques to nucleic acids that encode the protein and cells or organisms that express the protein. With respect to a nucleic acid, the term “recombinant” or “engineered” means having an altered nucleic acid sequence as a result of the application of genetic engineering techniques. Genetic engineering techniques include, but are not limited to, PCR and DNA cloning technologies; transfection, transformation, and other gene transfer technologies; homologous recombination; site-directed mutagenesis; and gene fusion. In accordance with this definition, a protein having an amino acid sequence identical to a naturally-occurring protein, but produced by cloning and expression in a heterologous host, is not considered recombinant or engineered.

As used herein, the term “recombinant DNA construct,” “recombinant construct,” “expression cassette,” “expression construct,” “chimeric construct,” “construct,” and “recombinant DNA fragment” are used interchangeably herein and are single or double-stranded polynucleotides. A recombinant construct comprises an artificial combination of nucleic acid fragments, including, without limitation, regulatory and coding sequences that are not found together in nature. For example, a recombinant DNA construct may comprise regulatory sequences and coding sequences that are derived from different sources, or regulatory sequences and coding sequences derived from the same source and arranged in a manner different than that found in nature. Such a construct may be used by itself or may be used in conjunction with a vector.

As used herein, the term “tissue-specific promoter” refers to a nucleotide sequence which, when operably linked with a polynucleotide encodes or specified by a gene, causes the gene product to be produced in a cell substantially only if the cell is a cell of the tissue type corresponding to the promoter.

As used herein, the terms “transfected” or “transformed” or “transduced” or “nucleofected” refer to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

As used herein, the term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

As used herein, the term “transient” refers to expression of a non-integrated transgene for a period of hours, days or weeks, wherein the period of time of expression is less than the period of time for expression of the gene if integrated into the genome or contained within a stable plasmid replicon in the host cell.

As used herein, the term “vector” or “recombinant DNA vector” may be a construct that includes a replication system and sequences that are capable of transcription and translation of a polypeptide-encoding sequence in a given host cell. If a vector is used, then the choice of vector is dependent upon the method that will be used to transform host cells as is well known to those skilled in the art. Vectors can include, without limitation, plasmid vectors and recombinant AAV vectors, or any other vector known in the art suitable for delivering a gene to a target cell. The skilled artisan is well aware of the genetic elements that must be present on the vector in order to successfully transform, select and propagate host cells comprising any of the isolated nucleotides or nucleic acid sequences of the invention.

In some embodiments, a “vector” also refers to a viral vector (i.e., a recombinant virus). Viral vectors can include, without limitation, retroviral vectors, lentiviral vectors, adenoviral vectors, and adeno-associated viral vectors (AAV).

As used herein, the term “wild-type” refers to the most common naturally occurring allele (i.e., polynucleotide sequence) in the allele population of the same type of gene, wherein a polypeptide encoded by the wild-type allele has its original functions. The term “wild-type” also refers to a polypeptide encoded by a wild-type allele. Wild-type alleles (i.e., polynucleotides) and polypeptides are distinguishable from mutant or variant alleles and polypeptides, which comprise one or more mutations and/or substitutions relative to the wild-type sequence(s). Whereas a wild-type allele or polypeptide can confer a normal phenotype in an organism, a mutant or variant allele or polypeptide can, in some instances, confer an altered phenotype. Wild-type nucleases are distinguishable from recombinant or non-naturally-occurring nucleases. The term “wild-type” can also refer to a cell, an organism, and/or a subject which possesses a wild-type allele of a particular gene, or a cell, an organism, and/or a subject used for comparative purposes.

As used herein, the term “altered specificity,” when referencing to a nuclease, means that a nuclease binds to and cleaves a recognition sequence, which is not bound to and cleaved by a reference nuclease (e.g., a wild-type) under physiological conditions, or that the rate of cleavage of a recognition sequence is increased or decreased by a biologically significant amount (e.g., at least 2×, or 2×-10×) relative to a reference nuclease.

As used herein, the term “center sequence” refers to the four base pairs separating half-sites in the meganuclease recognition sequence. These bases are numbered +1 through +4. The center sequence comprises the four bases that become the 3′ single-strand overhangs following meganuclease cleavage. “Center sequence” can refer to the sequence of the sense strand or the antisense (opposite) strand. Meganucleases are symmetric and recognize bases equally on both the sense and antisense strand of the center sequence. For example, the sequence A+1A+2A+3A+4 on the sense strand is recognized by a meganuclease as T+1T+2T+3T+4 on the antisense strand and, thus, A+1A+2A+3A+4 and T+1T+2T+3T+4 are functionally equivalent (e.g., both can be cleaved by a given meganuclease). Thus, the sequence C+1T+2G+3C+4, is equivalent to its opposite strand sequence, G+1C+2A+3G+4 due to the fact that the meganuclease binds its recognition sequence as a symmetric homodimer.

As used herein, the terms “cleave” or “cleavage” refer to the hydrolysis of phosphodiester bonds within the backbone of a recognition sequence within a target sequence that results in a double-stranded break within the target sequence, referred to herein as a “cleavage site”.

As used herein, the terms “DNA-binding affinity” or “binding affinity” means the tendency of a nuclease to non-covalently associate with a reference DNA molecule (e.g., a recognition sequence or an arbitrary sequence). Binding affinity is measured by a dissociation constant, Kd. As used herein, a nuclease has “altered” binding affinity if the Kd of the nuclease for a reference recognition sequence is increased or decreased by a statistically significant percent change relative to a reference nuclease.

As used herein, the term “hypervariable region” refers to a localized sequence within a meganuclease monomer or subunit that comprises amino acids with relatively high variability. A hypervariable region can comprise about 50-60 contiguous residues, about 53-57 contiguous residues, or preferably about 56 residues. In some embodiments, the residues of a hypervariable region may correspond to positions 24-79 or positions 215-270 of any one of SEQ ID NOs: 2 or 4. A hypervariable region can comprise one or more residues that contact DNA bases in a recognition sequence and can be modified to alter base preference of the monomer or subunit. A hypervariable region can also comprise one or more residues that bind to the DNA backbone when the meganuclease associates with a double-stranded DNA recognition sequence. Such residues can be modified to alter the binding affinity of the meganuclease for the DNA backbone and the target recognition sequence. In different embodiments of the invention, a hypervariable region may comprise between 1-20 residues that exhibit variability and can be modified to influence base preference and/or DNA-binding affinity. In particular embodiments, a hypervariable region comprises between about 15-20 residues that exhibit variability and can be modified to influence base preference and/or DNA-binding affinity. In some embodiments, variable residues within a hypervariable region correspond to one or more of positions 24, 26, 28, 30, 32, 33, 38, 40, 42, 44, 46, 68, 70, 75, and 77 of SEQ ID NO: 2. In other embodiments, variable residues within a hypervariable region correspond to one or more of positions 215, 217, 219, 221, 223, 224, 229, 231, 233, 235, 237, 259, 261, 266, and 268 of SEQ ID NO: 2.

As used herein, the term “linker” refers to an exogenous peptide sequence used to join two nuclease subunits into a single polypeptide. A linker may have a sequence that is found in natural proteins or may be an artificial sequence that is not found in any natural protein. A linker may be flexible and lacking in secondary structure or may have a propensity to form a specific three-dimensional structure under physiological conditions. A linker can include, without limitation, those encompassed by U.S. Pat. Nos. 8,445,251, 9,340,777, 9,434,931, and 10,041,053, each of which is incorporated by reference in its entirety.

As used herein, the term “meganuclease” refers to an endonuclease that binds double-stranded DNA at a recognition sequence that is greater than 12 base pairs. In some embodiments, the recognition sequence for a meganuclease of the present disclosure is 22 base pairs. A meganuclease can be an endonuclease that is derived from I-CreI (SEQ ID NO: 1), and can refer to an engineered variant of I-CreI that has been modified relative to natural I-CreI with respect to, for example, DNA-binding specificity, DNA cleavage activity, DNA-binding affinity, or dimerization properties. Methods for producing such modified variants of I-CreI are known in the art (e.g., WO 2007/047859, incorporated by reference in its entirety). A meganuclease as used herein binds to double-stranded DNA as a heterodimer. A meganuclease may also be a “single-chain meganuclease” in which a pair of DNA-binding domains is joined into a single polypeptide using a peptide linker. The term “homing endonuclease” is synonymous with the term “meganuclease.” Meganucleases of the present disclosure are substantially non-toxic when expressed in the targeted cells as described herein such that cells can be transfected and maintained at 37° C. without observing deleterious effects on cell viability or significant reductions in meganuclease cleavage activity when measured using the methods described herein.

As used herein, the term “mitochondria-targeting engineered meganuclease” or “MTEM” refers to an engineered meganuclease attached to a peptide or other molecule that is capable of directing the engineered meganuclease to the mitochondria such that the engineered meganuclease is capable of binding and cleaving mitochondrial DNA within the mitochondrial organelle. As used herein, the term MTEM is an example of an MTEN defined elsewhere herein.

As used herein the term “mitochondrial transit peptide” or “MTP” refers to a peptide or fragment of amino acids that can be attached to a separate molecule in order to transport the molecule in the mitochondria. For example, an MTP can be attached to a nuclease, such as an engineered meganuclease, in order to transport the engineered meganuclease into the mitochondria. MTPs can consist of an alternating pattern of hydrophobic and positively charged amino acids to form what is called amphipathic helix.

As used herein, the terms “nuclease” and “endonuclease” are used interchangeably to refer to naturally-occurring or engineered enzymes, which cleave a phosphodiester bond within a polynucleotide chain.

As used herein, the term “recognition half-site,” “recognition sequence half-site,” or simply “half-site” means a nucleic acid sequence in a double-stranded DNA molecule that is recognized and bound by a monomer of a homodimeric or heterodimeric meganuclease or by one subunit of a single-chain meganuclease.

As used herein, the terms “recognition sequence” or “recognition site” refers to a DNA sequence that is bound and cleaved by a nuclease. In the case of a meganuclease, a recognition sequence comprises a pair of inverted, 9 basepair “half sites” which are separated by four basepairs. In the case of a single-chain meganuclease, the N-terminal domain of the protein contacts a first half-site and the C-terminal domain of the protein contacts a second half-site. Cleavage by a meganuclease produces four basepair 3′ overhangs. “Overhangs,” or “sticky ends” are short, single-stranded DNA segments that can be produced by endonuclease cleavage of a double-stranded DNA sequence. In the case of meganucleases and single-chain meganucleases derived from I-CreI, the overhang comprises bases 10-13 of the 22 basepair recognition sequence. As used herein, the term “single-chain meganuclease” refers to a polypeptide comprising a pair of nuclease subunits joined by a linker. A single-chain meganuclease has the organization: N-terminal subunit—Linker—C-terminal subunit. The two meganuclease subunits will generally be non-identical in amino acid sequence and will bind non-identical DNA sequences. Thus, single-chain meganucleases typically cleave pseudo-palindromic or non-palindromic recognition sequences. A single-chain meganuclease may be referred to as a “single-chain heterodimer” or “single-chain heterodimeric meganuclease” although it is not, in fact, dimeric. For clarity, unless otherwise specified, the term “meganuclease” can refer to a dimeric or single-chain meganuclease.

As used herein, the term “specificity” means the ability of a nuclease to bind and cleave double-stranded DNA molecules only at a particular sequence of base pairs referred to as the recognition sequence, or only at a particular set of recognition sequences. The set of recognition sequences will share certain conserved positions or sequence motifs but may be degenerate at one or more positions. A highly-specific nuclease is capable of cleaving only one or a very few recognition sequences. Specificity can be determined by any method known in the art.

As used herein, the terms “target site” or “target sequence” refers to a region of the chromosomal DNA of a cell comprising a recognition sequence for a nuclease.

As used herein, the term “treatment” or “treating a subject” refers to the administration of an engineered nuclease of the invention, or a nucleic acid encoding an engineered nuclease of the invention. In some aspects, an engineered nuclease of the invention or a nucleic acid encoding the same is administered during treatment in the form of a pharmaceutical composition of the invention.

As used herein, a “vector” can also refer to a viral vector (i.e., a recombinant virus). Viral vectors can include, without limitation, retroviral vectors, lentiviral vectors, adenoviral vectors, and adeno-associated viral vectors (AAV).

As used herein, the term “serotype” or “capsid” refers to a distinct variant within a species of virus that is determined based on the viral cell surface antigens. Known serotypes of AAV include, among others, AAV1, AAV2, AAV3, AAV3B, AAV4, AAV5, AAV6, AAV7, AAV8, AAV9, AAV10, AAV11, and AAVHSC (Weitzman and Linden (2011) In Snyder and Moullier Adeno-associated virus methods and protocols. Totowa, NJ: Humana Press).

As used herein, a “control” or “control cell” refers to a cell that provides a reference point for measuring changes in genotype or phenotype of a genetically-modified cell. A control cell may comprise, for example: (a) a wild-type cell, i.e., of the same genotype as the starting material for the genetic alteration which resulted in the genetically-modified cell; (b) a cell of the same genotype as the genetically-modified cell but which has been transformed with a null construct (i.e., with a construct which has no known effect on the trait of interest); or, (c) a cell genetically identical to the genetically-modified cell but which is not exposed to conditions, stimuli, or further genetic modifications that would induce expression of altered genotype or phenotype.

As used herein, the term “effective amount” or “therapeutically effective amount” refers to an amount sufficient to effect beneficial or desirable biological and/or clinical results. The therapeutically effective amount will vary depending on the formulation or composition used, the disease and its severity and the age, weight, physical condition and responsiveness of the subject to be treated. In some specific embodiments, an effective amount of the MTEM comprises about 1×10¹⁰ gc/kg to about 1×10¹⁴ gc/kg (e.g., 1×10¹⁰ gc/kg, 1×10¹¹ gc/kg, 1×10¹² gc/kg, 1×10¹³ gc/kg, or 1×10¹⁴ gc/kg) of a nucleic acid encoding the MTEM or of a template nucleic acid. In specific embodiments, an effective amount of a nucleic acid encoding an MTEM and/or a template nucleic acid, or a pharmaceutical composition comprising a nucleic acid encoding an MTEM and/or a template nucleic acid disclosed herein, reduces at least one symptom of a disease in a subject.

As used herein, the term “effective dose”, “effective amount”, “therapeutically effective dose”, or “therapeutically effective amount,” as used herein, refers to an amount sufficient to effect beneficial or desirable biological and/or clinical results.

As used herein, the term “gc/kg” or “gene copies/kilogram” refers to the number of copies of a nucleic acid encoding an MTEM or the number of copies of a template nucleic acid described herein per weight in kilograms of a subject that is administered the nucleic acid encoding the MTEM and/or the template nucleic acid.

As used herein, the term “preventing” refers to the prevention of the disease or condition in the patient.

As used herein, the term “prophylaxis” means the prevention of or protective treatment for a disease or disease state.

As used herein, the term “reduced” refers to any reduction in the symptoms or severity of a disease. Such a reduction may be up to 5%, 10%, 20%, 30%, 40%, 50%, 60%, 70%, 80%, 90%, 95%, or up to 100%. Accordingly, the term “reduced” encompasses both a partial reduction and a complete reduction of a disease state.

As used herein, the recitation of a numerical range for a variable is intended to convey that the present disclosure may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values ≥0 and ≤2 if the variable is inherently continuous.

### 2.1 Principle of the Invention

Mitochondria regulate cellular energy and metabolism under normal growth and development, as well as in response to stress. Thus, editing of the mitochondrial genome has diverse applications in both animals and plants. In humans, deleterious mitochondrial mutations are the source of a number of disorders for which gene editing therapies could be applied. However, albeit the potentials of using mitochondrial genome editing for therapeutic applications, it still remains an underexplored area of science because of the inability to efficiently target mitochondrial DNA (mtDNA) and generate precise edits. The mitochondrial genome is difficult to edit as the editing technology needs to be delivered to this organelle. Also, the mitochondria lack predictable repair mechanisms. Previous attempts at editing the mitochondrial genome have resulted in large and unpredictable deletions/rearrangements. Hence, compositions and methods that would allow targeting and editing defined regions (preferably limited to just one gene) of the mitochondrial genome in a more predictable manner are desired.

The present disclosure provides compositions and methods for binding and cleaving a recognition sequence on the mitochondrial genome without impacting the surrounding regions in the mitochondrial genome. Disclosed herein are engineered nucleases, such as engineered meganucleases, attached to MTPs such that DSBs can be generated in the mtDNA. The present invention demonstrates that engineered meganucleases can be directed into the mitochondria organelle and facilitate precise editing of mtDNA, thus opening up an entire field of prospects and opportunities in life sciences.

### 2.2 Mitochondria-Targeting Engineered Meganuclease for Recognizing and Cleaving Recognition Sequences within the Human Mitochondrial DNA

Mitochondria-targeting engineered meganucleases (MTEM) constructed of an engineered meganuclease attached to a mitochondrial transit peptide (MTP) can effectively traffic from the cytoplasm of a eukaryotic cell into the mitochondria. Once inside the mitochondria organelle, the MTEM can bind and cleave a recognition sequence in the mitochondrial genome. It is known in the art that it is possible to use a site-specific nuclease to make a DNA break in the genome of a living cell, and that such a DNA break can result in permanent modification of the genome via mutagenic NHEJ repair or via homologous recombination with a transgenic DNA sequence. NHEJ can produce mutagenesis at the cleavage site, resulting in inactivation of the allele. NHEJ-associated mutagenesis may inactivate an allele via generation of early stop codons, frameshift mutations producing aberrant non-functional proteins, or could trigger mechanisms such as nonsense-mediated mRNA decay. The use of nucleases to induce mutagenesis via NHEJ can be used to target a specific mutation or a sequence present in a wild-type allele. Further, the use of nucleases to induce a double-strand break in a target locus is known to stimulate homologous recombination, particularly of transgenic DNA sequences flanked by sequences that are homologous to the genomic target. In this manner, exogenous nucleic acid sequences can be inserted into a target locus. Such exogenous nucleic acids can encode any sequence or polypeptide of interest.

The nucleases used to practice the invention are meganucleases. In particular embodiments, the meganucleases used to practice the invention are single-chain meganucleases. A single-chain meganuclease comprises an N-terminal subunit and a C-terminal subunit joined by a linker peptide. Each of the two domains recognizes and binds to half of the recognition sequence (i.e., a recognition half-site) and the site of DNA cleavage is at the middle of the recognition sequence near the interface of the two subunits. DNA strand breaks are offset by four base pairs such that DNA cleavage by a meganuclease generates a pair of four base pair, 3′ single-strand overhangs.

In some embodiments, an engineered meganuclease of the invention has been engineered to bind and cleave an MIT 11-12 recognition sequence (SEQ ID NO: 3). Such engineered meganuclease is referred to herein as “MIT 11-12 meganuclease” or “MIT 11-12 nuclease”.

Engineered meganucleases of the invention can comprise a first subunit, comprising a first hypervariable (HVR1) region, and a second subunit, comprising a second hypervariable (HVR2) region. Further, the first subunit can bind to a first recognition half-site in the recognition sequence and the second subunit can bind to a second recognition half-site in the recognition sequence. In embodiments where the engineered meganuclease is a single-chain meganuclease, the first and second subunits can be oriented such that the first subunit, which comprises the HVR1 region and binds the first half-site, is positioned as the N-terminal subunit, and the second subunit, which comprises the HVR2 region and binds the second half-site, is positioned as the C-terminal subunit. In alternative embodiments, the first and second subunits can be oriented such that the first subunit, which comprises the HVR1 region and binds the first half-site, is positioned as the C-terminal subunit, and the second subunit, which comprises the HVR2 region and binds the second half-site, is positioned as the N-terminal subunit.

In various embodiments, the first and/or second subunits of the engineered meganuclease comprised by the MTEM comprise an amino acid sequence having at least 80%, at least 85%, at least 90%, at least 91%, at least 92%, at least 93%, at least 94%, at least 95%, at least 96%, at least 97%, at least 98%, or at least 99% sequence identity to a sequence set forth in SEQ ID NO: 1. In certain embodiments, the first and/or second subunits of the engineered meganuclease comprised by the MTEM comprise an amino acid sequence having at least 80%, at least 85%, at least 90%, at least 91%, at least 92%, at least 93%, at least 94%, at least 95%, at least 96%, at least 97%, at least 98%, or at least 99% sequence identity to residues 7-153 of SEQ ID NO: 1. In further embodiments, the engineered meganuclease comprised by the MTEM comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 2. In certain embodiments of the invention, the engineered meganuclease binds and cleaves a recognition sequence comprising SEQ ID NO: 3 within the mitochondrial genome, wherein the engineered meganuclease comprises a first subunit and a second subunit, wherein the first subunit binds to a first recognition half-site of the recognition sequence and comprises a first hypervariable (HVR1) region, and wherein the second subunit binds to a second recognition half-site of the recognition sequence and comprises a second hypervariable (HVR2) region.

In some embodiments, the HVR1 region comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to an amino acid sequence corresponding to residues 24-79 of SEQ ID NO: 2. In some such embodiments, the HVR1 region comprises one or more residues corresponding to residues 24, 26, 28, 30, 32, 33, 38, 40, 42, 44, 46, 68, 70, 75, and 77 of SEQ ID NO: 2. In some such embodiments, the HVR1 region comprises residues corresponding to residues 24, 26, 28, 30, 32, 33, 38, 40, 42, 44, 46, 68, 70, 75, and 77 of SEQ ID NO: 2. In some such embodiments, the HVR1 region comprises Y, R, K, or D at a residue corresponding to residue 66 of SEQ ID NO: 2. In some such embodiments, the HVR1 region comprises residues 24-79 of SEQ ID NO: 2. In some such embodiments, the HVR2 region comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to an amino acid sequence corresponding to residues 215-270 of SEQ ID NO: 2. In some such embodiments, the HVR2 region comprises one or more residues corresponding to residues 215, 217, 219, 221, 223, 224, 229, 231, 233, 235, 237, 259, 261, 266, and 268 of SEQ ID NO: 2. In some such embodiments, the HVR2 region comprises residues corresponding to residues 215, 217, 219, 221, 223, 224, 229, 231, 233, 235, 237, 259, 261, 266, and 268 of SEQ ID NO: 2. In some such embodiments, the HVR2 region comprises Y, R, K, or D at a residue corresponding to residue 257 of SEQ ID NO: 2. In some such embodiments, the HVR2 region comprises residues 215-270 of SEQ ID NO: 2. In some such embodiments, the first subunit comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to residues 7-153 of SEQ ID NO: 2, and wherein the second subunit comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to residues 198-344 of SEQ ID NO: 2. In some such embodiments, the first subunit comprises G, S, or A at a residue corresponding to residue 19 of SEQ ID NO: 2. In some such embodiments, the first subunit comprises E, Q, or K at a residue corresponding to residue 80 of SEQ ID NO: 2. In some such embodiments, the second subunit comprises G, S, or A at a residue corresponding to residue 210 of SEQ ID NO: 2. In some such embodiments, the second subunit comprises E, Q, or K at a residue corresponding to residue 271 of SEQ ID NO: 2. In some such embodiments, the first subunit comprises a residue corresponding to residue 80 of SEQ ID NO: 2. In some such embodiments, the second subunit comprises a residue corresponding to residue 271 of SEQ ID NO: 2. In some such embodiments, the engineered meganuclease is a single-chain meganuclease comprising a linker, wherein the linker covalently joins the first subunit and the second subunit. In some such embodiments, the engineered meganuclease comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 2. In some such embodiments, the engineered meganuclease comprises the amino acid sequence of SEQ ID NO: 2. In some embodiments, the engineered meganuclease is encoded by a nucleic sequence having at least 80%, at least 85%, at least 90%, at least 91%, at least 92%, at least 93%, at least 94%, at least 95%, at least 96%, at least 97%, at least 98%, or at least 99% sequence identity to the nucleic acid sequence set forth in SEQ ID NO: 3. In some such embodiments, the engineered meganuclease is encoded by the nucleic acid sequence set forth in SEQ ID NO: 3.

MTPs for directing the engineered meganuclease into the mitochondria can be from 10-100 amino acids in length. In specific embodiments, the MTP is about 10, about 11, about 12, about 13, about 14, about 15, about 16, about 17, about 18, about 19, about 20, about 21, about 22, about 23, about 24, about 25, about 26, about 27, about 28, about 29, about 30, about 35, about 40, about 45, about 50, about 55, about 60, about 65, about 70, about 75, about 80, about 85, about 90, about 95, about 100, or more amino acids long. MTPs can contain additional signals that subsequently target the protein to different regions of the mitochondria, such as the mitochondrial matrix. Non limiting examples of MTPs for use in the compositions and methods disclose herein include, Neurospora crassa F0 ATPase subunit 9 (SU9) MTP, human cytochrome c oxidase subunit VIII (CoxVIII or Cox8) MTP, the P1 isoform of subunit c of human ATP synthase MTP, aldehyde dehydrogenase targeting sequence MTP, Glutaredoxin 5 MTP, Pyruvate dehydrogenase MTP, Peptidyl-prolyl isomerase MTP, Acetyltransferase MTP, Isocitrate dehydrogenase MTP, cytochrome oxidase MTP, and the subunits of the FA portion of ATP synthase MTP, CPN60/No GGlinker MTP, Superoxide dismutase (SOD) MTP, Superoxide dismutase doubled(2SOD) MTP, Superoxide dismutase modified(SODmod) MTP, Superoxide dismutase modified (2SODmod) doubled MTP, L29 MTP, gATPase gamma subunit (FAγ51) MTP, CoxIV twin strep (ABM97483) MTP, and CoxIV 10×His MTP.

In specific embodiments, the MTP comprises a combination of at least two MTPs. The combination of MTPs can be a combination of identical MTPs or a combination of different MTPs. In specific embodiments, the MTP comprises the Cox VIII MTP (SEQ ID NO: 6) and the SU9 MTP (SEQ ID NO: 7) into a single MTP represented by SEQ ID NO: 8.

In order to form an MTEM, an MTP can be attached by any appropriate means to an engineered meganuclease disclosed herein. In specific embodiments, the MTP can be attached to the N-terminus of the engineered meganuclease. In other embodiments the MTP can be attached to the C-terminus of the engineered meganuclease. In some embodiments multiple MTPs can be attached to a single engineered meganuclease to form an MTEM. For example, a first MTP can be attached to the N-terminus of the engineered meganuclease and a second MTP can be attached to the C-terminus of the MTP. In some embodiments, the first and second MTP are identical and is other embodiments, the first and second MTP not identical. The MTP(s) can be attached by any means that allows for transport of the engineered meganuclease into the mitochondria of a cell. In specific embodiments, the MTP is attached by fusing the MTP to the N- or C-terminus of the engineered meganuclease. The MTP can also be attached to the engineered meganuclease by a peptide linker. The linker can be, for example, about 1, 2, 3, 4, 5, 6, 7, 8, 9, 10, 11, 12, 15, or 20 amino acids. In specific embodiments the MTP is attached to a peptide linker at the N- or C-terminus of the engineered meganuclease.

In some embodiments, an MTEM for use in the compositions and methods of the present disclosure is attached to a nuclear export sequence (NES) in order to help prevent the MTEM from cleaving the nuclear genome. In some such embodiments, the NES comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 9 or 10. For example, the NES may comprise the amino acid sequence of SEQ ID NO: 9 or 10. In certain embodiments, the NES is attached at the N-terminus of the engineered meganuclease. In other embodiments, the NES is attached at the C-terminus of the engineered meganuclease. In certain embodiments, the NES is fused to the engineered meganuclease. In certain embodiments, the NES is attached to the engineered meganuclease by a polypeptide linker.

In specific embodiments, the MTEM is attached to multiple NESs. For example, an engineered meganuclease disclosed herein can comprise a first NES and a second NES. In some such embodiments, the first NES is attached at the N-terminus of the MTEM, and the second NES is attached at the C-terminus of the MTEM. In some such embodiments, the first NES and/or the second NES comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 9 or 10. For example, the first NES and/or the second NES may comprise the amino acid sequence set forth in SEQ ID NO: 9 or 10. In some embodiments, the first NES and the second NES are identical. In other embodiments, the first NES and the second NES are not identical. The NES can be attached to the MTEM by any appropriate means known in the art. For example, the first NES and/or the second NES can be fused to the MTEM. In some embodiments, the first NES and/or the second NES is attached to the MTEM by a polypeptide linker.

An MTEM with an NES may have reduced or decreased transport to the nucleus of a target cell or target cell population (e.g., a eukaryotic cell or eukaryotic cell population), compared to an engineered meganuclease without an NES. For example, nuclear transport of an MTEM with an NES may be less than that of an MTEM without an NES, by about 5-10%, 10-20%, 20-30%, 30-40%, 40-50%, 50-60%, 60-70%, 70-80%, 80-90%, 90-100%, or more (e.g., by about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, 100%, or more). In some embodiments, an engineered meganuclease with an NES may induce fewer nuclear indels (i.e., less cleavage and resulting deletion in nuclear genome of a target cell or target cell population) compared to an MTEM without an NES. For example, nuclear indels induced by an MTEM with an NES may be less than that induced by an MTEM without an NES, by about 5-10%, 10-20%, 20-30%, 30-40%, 40-50%, 50-60%, 60-70%, 70-80%, 80-90%, 90-100%, or more.

### 2.3 Pharmaceutical Compositions

In some embodiments, the invention provides a pharmaceutical composition comprising a pharmaceutically acceptable carrier and MTEM of the invention, or a pharmaceutically acceptable carrier and a polynucleotide comprising a nucleic acid sequence encoding an MTEM of the invention. In particular, pharmaceutical compositions are provided that comprise a pharmaceutically acceptable carrier and a therapeutically effective amount of a nucleic acid encoding an MTEM, wherein the engineered meganuclease of the MTEM has specificity for a recognition sequence within mtDNA, such as human mtDNA.

In other embodiments, the invention provides a pharmaceutical composition comprising a pharmaceutically acceptable carrier and a genetically-modified cell of the invention.

Such pharmaceutical compositions can be prepared in accordance with known techniques. See, e.g., Remington, The Science And Practice of Pharmacy (21st ed., Philadelphia, Lippincott, Williams & Wilkins, 2005). In the manufacture of a pharmaceutical formulation according to the invention, nuclease polypeptides (or DNA/RNA encoding the same or cells expressing the same) are typically admixed with a pharmaceutically acceptable carrier, and the resulting composition is administered to a subject. The carrier must be acceptable in the sense of being compatible with any other ingredients in the formulation and must not be deleterious to the subject. In some embodiments, pharmaceutical compositions of the invention can further comprise one or more additional agents or biological molecules useful in the treatment of a disease in the subject. Likewise, the additional agent(s) and/or biological molecule(s) can be co-administered as a separate composition.

In particular embodiments of the invention, the pharmaceutical composition comprises a recombinant virus (i.e., a viral vector) comprising a polynucleotide (e.g., a viral genome) comprising a nucleic acid sequence encoding an MTEM described herein. Such recombinant viruses are known in the art and include recombinant retroviruses, recombinant lentiviruses, recombinant adenoviruses, and recombinant adeno-associated viruses (AAV) (reviewed in Vannucci, et al. (2013 New Microbiol. 36:1-22). Recombinant AAVs useful in the invention can have any capsid or serotype that allows for transduction of the virus into a target cell type and expression of the MTEM by the target cell. For example, in some embodiments, the recombinant AAV has a serotype of AAV1, AAV2, AAV3, AAV3B, AAV4, AAV5, AAV6, AAV7, AAV8, AAV9, AAV10, AAV11, or AAVHSC. In some embodiments, the recombinant virus is injected directly into target tissues. In alternative embodiments, the recombinant virus is delivered systemically via the circulatory system. It is known in the art that different AAVs tend to localize to different tissues, and one could select an appropriate AAV capsid/serotype for preferential delivery to a particular tissue. Accordingly, in some embodiments, the AAV serotype is AAV2. In alternative embodiments, the AAV serotype is AAV6. In other embodiments, the AAV serotype is AAV8. In still other embodiments, the AAV serotype is AAV9. In further embodiments, the AAV serotype is AAHSC. AAV vectors can also be self-complementary such that they do not require second-strand DNA synthesis in the host cell (McCarty, et al. (2001) Gene Ther. 8:1248-54). Nucleic acids delivered by recombinant AAV vectors can include left (5′) and right (3′) inverted terminal repeats.

In particular embodiments of the invention, the pharmaceutical composition comprises one or more mRNAs described herein (e.g., mRNAs encoding MTEMs) formulated within lipid nanoparticles.

The selection of cationic lipids, non-cationic lipids and/or lipid conjugates which comprise the lipid nanoparticle, as well as the relative molar ratio of such lipids to each other, is based upon the characteristics of the selected lipid(s), the nature of the intended target cells, and the characteristics of the mRNA to be delivered. Additional considerations include, for example, the saturation of the alkyl chain, as well as the size, charge, pH, pKa, fusogenicity and toxicity of the selected lipid(s). Thus, the molar ratios of each individual component may be adjusted accordingly.

The lipid nanoparticles for use in the method of the invention can be prepared by various techniques which are presently known in the art. Nucleic acid-lipid particles and their method of preparation are disclosed in, for example, U.S. Patent Publication Nos. 20040142025 and 20070042031, the disclosures of which are herein incorporated by reference in their entirety for all purposes.

Selection of the appropriate size of lipid nanoparticles must take into consideration the site of the target cell and the application for which the lipid nanoparticles is being made. Generally, the lipid nanoparticles will have a size within the range of about 25 to about 500 nm. In some embodiments, the lipid nanoparticles have a size from about 50 nm to about 300 nm or from about 60 nm to about 120 nm. The size of the lipid nanoparticles may be determined by quasi-electric light scattering (QELS) as described in Bloomfield, Ann. Rev. Biophys. Bioeng., 10:421-150 (1981), incorporated herein by reference. A variety of methods are known in the art for producing a population of lipid nanoparticles of particular size ranges, for example, sonication or homogenization. One such method is described in U.S. Pat. No. 4,737,323, incorporated herein by reference.

Some lipid nanoparticles contemplated for use in the invention comprise at least one cationic lipid, at least one non-cationic lipid, and at least one conjugated lipid. In more particular examples, lipid nanoparticles can comprise from about 50 mol % to about 85 mol % of a cationic lipid, from about 13 mol % to about 49.5 mol % of a non-cationic lipid, and from about 0.5 mol % to about 10 mol % of a lipid conjugate, and are produced in such a manner as to have a non-lamellar (i.e., non-bilayer) morphology. In other particular examples, lipid nanoparticles can comprise from about 40 mol % to about 85 mol % of a cationic lipid, from about 13 mol % to about 49.5 mol % of a non-cationic lipid, and from about 0.5 mol % to about 10 mol % of a lipid conjugate, and are produced in such a manner as to have a non-lamellar (i.e., non-bilayer) morphology.

Cationic lipids can include, for example, one or more of the following: palmitoyi-oleoyl-nor-arginine (PONA), MPDACA, GUADACA, ((6Z,9Z,28Z,31Z)-heptatriaconta-6,9,28,31-tetraen-19-yl 4-(dimethylamino)butanoate) (MC3), LenMC3, CP-LenMC3, γ-LenMC3, CP-γ-LenMC3, MC3MC, MC2MC, MC3 Ether, MC4 Ether, MC3 Amide, Pan-MC3, Pan-MC4 and Pan MC5, 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)-[1,3]-dioxolane (DLin-K-C2-DMA; “XTC2”), 2,2-dilinoleyl-4-(3-dimethylaminopropyl)-[1,3]-dioxolane (DLin-K-C3-DMA), 2,2-dilinoleyl-4-(4-dimethylaminobutyl)-[1,3]-dioxolane (DLin-K-C4-DMA), 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 2,2-dilinoleyl-4-dimethylaminomethyl-[1,3]-dioxolane (DLin-K-DMA), 1,2-dilinoleylcarbamoyloxy-3-dimethylaminopropane (DLin-C-DAP), 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.CI), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino)ethoxypropane (DLin-EG-DMA), N,N-dioleyl-N,N-dimethylammonium chloride (DODAC), 1,2-dioleyloxy-N,N-dimethylaminopropane (DODMA), 1,2-distearyloxy-N,N-dimethylaminopropane (DSDMA), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N—(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2(spermine-carboxamido)ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethy-1-(cis,cis-9′, 1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-dilinoleylcarbamyl-3-dimethylaminopropane (DLincarbDAP), or mixtures thereof. The cationic lipid can also be DLinDMA, DLin-K-C2-DMA (“XTC2”), MC3, LenMC3, CP-LenMC3, γ-LenMC3, CP-γ-LenMC3, MC3MC, MC2MC, MC3 Ether, MC4 Ether, MC3 Amide, Pan-MC3, Pan-MC4, Pan MC5, or mixtures thereof.

In various embodiments, the cationic lipid comprises from about 50 mol % to about 90 mol %, from about 50 mol % to about 85 mol %, from about 50 mol % to about 80 mol %, from about 50 mol % to about 75 mol %, from about 50 mol % to about 70 mol %, from about 50 mol % to about 65 mol %, or from about 50 mol % to about 60 mol % of the total lipid present in the particle.

In other embodiments, the cationic lipid comprises from about 40 mol % to about 90 mol %, from about 40 mol % to about 85 mol %, from about 40 mol % to about 80 mol %, from about 40 mol % to about 75 mol %, from about 40 mol % to about 70 mol %, from about 40 mol % to about 65 mol %, or from about 40 mol % to about 60 mol % of the total lipid present in the particle.

The non-cationic lipid may comprise, e.g., one or more anionic lipids and/or neutral lipids. In particular embodiments, the non-cationic lipid comprises one of the following neutral lipid components: (1) cholesterol or a derivative thereof; (2) a phospholipid; or (3) a mixture of a phospholipid and cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, and mixtures thereof. The phospholipid may be a neutral lipid including, but not limited to, dipalmitoylphosphatidylcholine (DPPC), distearoylphosphatidylcholine (DSPC), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC), and mixtures thereof. In certain particular embodiments, the phospholipid is DPPC, DSPC, or mixtures thereof.

In some embodiments, the non-cationic lipid (e.g., one or more phospholipids and/or cholesterol) comprises from about 10 mol % to about 60 mol %, from about 15 mol % to about 60 mol %, from about 20 mol % to about 60 mol %, from about 25 mol % to about 60 mol %, from about 30 mol % to about 60 mol %, from about 10 mol % to about 55 mol %, from about 15 mol % to about 55 mol %, from about 20 mol % to about 55 mol %, from about 25 mol % to about 55 mol %, from about 30 mol % to about 55 mol %, from about 13 mol % to about 50 mol %, from about 15 mol % to about 50 mol % or from about 20 mol % to about 50 mol % of the total lipid present in the particle. When the non-cationic lipid is a mixture of a phospholipid and cholesterol or a cholesterol derivative, the mixture may comprise up to about 40, 50, or 60 mol % of the total lipid present in the particle.

The conjugated lipid that inhibits aggregation of particles may comprise, e.g., one or more of the following: a polyethyleneglycol (PEG)-lipid conjugate, a polyamide (ATTA)-lipid conjugate, a cationic-polymer-lipid conjugates (CPLs), or mixtures thereof. In one particular embodiment, the nucleic acid-lipid particles comprise either a PEG-lipid conjugate or an ATTA-lipid conjugate. In certain embodiments, the PEG-lipid conjugate or ATTA-lipid conjugate is used together with a CPL. The conjugated lipid that inhibits aggregation of particles may comprise a PEG-lipid including, e.g., a PEG-diacylglycerol (DAG), a PEG dialkyloxypropyl (DAA), a PEG-phospholipid, a PEG-ceramide (Cer), or mixtures thereof. The PEG-DAA conjugate may be PEG-di lauryloxypropyl (C12), a PEG-dimyristyloxypropyl (C14), a PEG-dipalmityloxypropyl (C16), a PEG-distearyloxypropyl (C18), or mixtures thereof.

Additional PEG-lipid conjugates suitable for use in the invention include, but are not limited to, mPEG2000-1,2-di-O-alkyl-sn3-carbomoylglyceride (PEG-C-DOMG). The synthesis of PEG-C-DOMG is described in PCT Application No. PCT/US08/88676. Yet additional PEG-lipid conjugates suitable for use in the invention include, without limitation, 1-[8′-(1,2-dimyristoyl-3-propanoxy)-carboxamido-3′,6′-dioxaoctanyl]carbamoyl-ωthyl-poly(ethylene glycol) (2KPEG-DMG). The synthesis of 2KPEG-DMG is described in U.S. Pat. No. 7,404,969.

In some cases, the conjugated lipid that inhibits aggregation of particles (e.g., PEG-lipid conjugate) may comprise from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 1 mol % to about 1.8 mol %, from about 1.2 mol % to about 1.8 mol %, from about 1.2 mol % to about 1.7 mol %, from about 1.3 mol % to about 1.6 mol %, from about 1.4 mol % to about 1.5 mol %, or about 1, 1.1, 1.2, 1.3, 1.4, 1.5, 1.6, 1.7, 1.8, 1.9, or 2 mol % (or any fraction thereof or range therein) of the total lipid present in the particle. Typically, in such instances, the PEG moiety has an average molecular weight of about 2,000 Daltons. In other cases, the conjugated lipid that inhibits aggregation of particles (e.g., PEG-lipid conjugate) may comprise from about 5.0 mol % to about 10 mol %, from about 5 mol % to about 9 mol %, from about 5 mol % to about 8 mol %, from about 6 mol % to about 9 mol %, from about 6 mol % to about 8 mol %, or about 5 mol %, 6 mol %, 7 mol %, 8 mol %, 9 mol %, or 10 mol % (or any fraction thereof or range therein) of the total lipid present in the particle. Typically, in such instances, the PEG moiety has an average molecular weight of about 750 Daltons.

In other embodiments, the composition comprises amphoteric liposomes, which contain at least one positive and at least one negative charge carrier, which differs from the positive one, the isoelectric point of the liposomes being between 4 and 8. This objective is accomplished owing to the fact that liposomes are prepared with a pH-dependent, changing charge.

Liposomal structures with the desired properties are formed, for example, when the amount of membrane-forming or membrane-based cationic charge carriers exceeds that of the anionic charge carriers at a low pH and the ratio is reversed at a higher pH. This is always the case when the ionizable components have a pKa value between 4 and 9. As the pH of the medium drops, all cationic charge carriers are charged more and all anionic charge carriers lose their charge.

Cationic compounds useful for amphoteric liposomes include those cationic compounds previously described herein above. Without limitation, strongly cationic compounds can include, for example: DC-Chol 3-β-[N-(N′,N′-dimethylmethane) carbamoyl] cholesterol, TC-Chol 3-βN-(N′, N′, N′-trimethylaminoethane) carbamoyl cholesterol, BGSC bisguanidinium-spermidine-cholesterol, BGTC bis-guadinium-tren-cholesterol, DOTAP (1,2-dioleoyloxypropyl)-N,N,N-trimethylammonium chloride, DOSPER (1,3-dioleoyloxy-2-(6-carboxy-spermyl)-propylarnide, DOTMA (1,2-dioleoyloxypropyl)-N,N,N-trimethylamronium chloride) (Lipofectin®), DORIE 1,2-dioleoyloxypropyl)-3-dimethylhydroxyethylammonium bromide, DOSC (1,2-dioleoyl-3-succinyl-sn-glyceryl choline ester), DOGSDSO (1,2-dioleoyl-sn-glycero-3-succinyl-2-hydroxyethyl disulfide omithine), DDAB dimethyldioctadecylammonium bromide, DOGS ((C18)2GlySper3+) N,N-dioctadecylamido-glycol-spermin (Transfectam®) (C18)2Gly+N,N-dioctadecylamido-glycine, CTAB cetyltrimethylarnmonium bromide, CpyC cetylpyridinium chloride, DOEPC 1,2-dioleoly-sn-glycero-3-ethylphosphocholine or other O-alkyl-phosphatidylcholine or ethanolamines, amides from lysine, arginine or ornithine and phosphatidyl ethanolamine.

Examples of weakly cationic compounds include, without limitation: His-Chol (histaminyl-cholesterol hemisuccinate), Mo-Chol (morpholine-N-ethylamino-cholesterol hemisuccinate), or histidinyl-PE.

Examples of neutral compounds include, without limitation: cholesterol, ceramides, phosphatidyl cholines, phosphatidyl ethanolamines, tetraether lipids, or diacyl glycerols.

Anionic compounds useful for amphoteric liposomes include those non-cationic compounds previously described herein. Without limitation, examples of weakly anionic compounds can include: CHEMS (cholesterol hemisuccinate), alkyl carboxylic acids with 8 to 25 carbon atoms, or diacyl glycerol hemisuccinate. Additional weakly anionic compounds can include the amides of aspartic acid, or glutamic acid and PE as well as PS and its amides with glycine, alanine, glutamine, asparagine, serine, cysteine, threonine, tyrosine, glutamic acid, aspartic acid or other amino acids or aminodicarboxylic acids. According to the same principle, the esters of hydroxycarboxylic acids or hydroxydicarboxylic acids and PS are also weakly anionic compounds.

In some embodiments, amphoteric liposomes contain a conjugated lipid, such as those described herein above. Particular examples of useful conjugated lipids include, without limitation, PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Some particular examples are PEG-modified diacylglycerols and dialkylglycerols.

In some embodiments, the neutral lipids comprise from about 10 mol % to about 60 mol %, from about 15 mol % to about 60 mol %, from about 20 mol % to about 60 mol %, from about 25 mol % to about 60 mol %, from about 30 mol % to about 60 mol %, from about 10 mol % to about 55 mol %, from about 15 mol % to about 55 mol %, from about 20 mol % to about 55 mol %, from about 25 mol % to about 55 mol %, from about 30 mol % to about 55 mol %, from about 13 mol % to about 50 mol %, from about 15 mol % to about 50 mol % or from about 20 mol % to about 50 mol % of the total lipid present in the particle.

In some cases, the conjugated lipid that inhibits aggregation of particles (e.g., PEG-lipid conjugate) comprises from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 1 mol % to about 1.8 mol %, from about 1.2 mol % to about 1.8 mol %, from about 1.2 mol % to about 1.7 mol %, from about 1.3 mol % to about 1.6 mol %, from about 1.4 mol % to about 1.5 mol %, or about 1, 1.1, 1.2, 1.3, 1.4, 1.5, 1.6, 1.7, 1.8, 1.9, or 2 mol % (or any fraction thereof or range therein) of the total lipid present in the particle. Typically, in such instances, the PEG moiety has an average molecular weight of about 2,000 Daltons. In other cases, the conjugated lipid that inhibits aggregation of particles (e.g., PEG-lipid conjugate) may comprise from about 5.0 mol % to about 10 mol %, from about 5 mol % to about 9 mol %, from about 5 mol % to about 8 mol %, from about 6 mol % to about 9 mol %, from about 6 mol % to about 8 mol %, or about 5 mol %, 6 mol %, 7 mol %, 8 mol %, 9 mol %, or 10 mol % (or any fraction thereof or range therein) of the total lipid present in the particle. Typically, in such instances, the PEG moiety has an average molecular weight of about 750 Daltons.

Considering the total amount of neutral and conjugated lipids, the remaining balance of the amphoteric liposome can comprise a mixture of cationic compounds and anionic compounds formulated at various ratios. The ratio of cationic to anionic lipid may selected in order to achieve the desired properties of nucleic acid encapsulation, zeta potential, pKa, or other physicochemical property that is at least in part dependent on the presence of charged lipid components.

In some embodiments, the lipid nanoparticles have a composition that specifically enhances delivery and uptake in a eukaryotic cell, such as a mammalian cell (e.g., a human cell). In certain embodiments, the lipid nanoparticles have a composition that specifically enhances delivery and uptake in the liver or specifically within hepatocytes. In certain embodiments, the lipid nanoparticles have a composition that specifically enhances delivery and uptake in a nerve cell.

### 2.4 Methods for Producing Recombinant Viruses

In some embodiments, the invention provides recombinant viruses (e.g., recombinant AAVs) for use in the methods of the invention. Recombinant AAVs are typically produced in mammalian cell lines such as HEK-293. Because the viral cap and rep genes are removed from the vector to prevent its self-replication to make room for the therapeutic gene(s) to be delivered (e.g. the nuclease gene), it is necessary to provide these in trans in the packaging cell line. In addition, it is necessary to provide the “helper” (e.g., adenoviral) components necessary to support replication (Cots et al. (2013), Curr. Gene Ther. 13(5): 370-81). Frequently, recombinant AAVs are produced using a triple-transfection in which a cell line is transfected with a first plasmid encoding the “helper” components, a second plasmid comprising the cap and rep genes, and a third plasmid comprising the viral ITRs containing the intervening DNA sequence to be packaged into the virus. Viral particles comprising a genome (ITRs and intervening gene(s) of interest) encased in a capsid are then isolated from cells by freeze-thaw cycles, sonication, detergent, or other means known in the art. Particles are then purified using cesium-chloride density gradient centrifugation or affinity chromatography and subsequently delivered to the gene(s) of interest to cells, tissues, or an organism such as a human patient.

Because recombinant AAVs are typically produced (manufactured) in cells, precautions must be taken in practicing the current invention to ensure that the MTEM is not expressed in the packaging cells. Because the viral genomes of the invention may comprise a recognition sequence for the nuclease, any nuclease expressed in the packaging cell line may be capable of cleaving the viral genome before it can be packaged into viral particles. This will result in reduced packaging efficiency and/or the packaging of fragmented genomes. Several approaches can be used to prevent nuclease expression in the packaging cells.

The MTEM can be placed under the control of any promoter suitable for expression of the MTEM. In some embodiments, the promoter is a constitutive promoter, or the promoter is a tissue-specific promoter such as, for example, a muscle cell-specific promoter, a skeletal muscle-specific promoter, a myotube-specific promoter, a muscle satellite cell-specific promoter, a neuron-specific promoter, an astrocyte-specific promoter, a microglia-specific promoter, an eye cell-specific promoter, a retinal cell-specific promoter, a retinal ganglion cell-specific promoter, a retinal pigmentary epithelium-specific promoter, a pancreatic cell-specific promoter, or a pancreatic beta cell-specific promoter. In some embodiments, the constitutive promoter is a CMV promoter, a CAG promoter, an EF1 alpha promoter, or a UbC promoter.

In specific embodiments, the MTEM can be placed under control of a tissue-specific promoter that is not active in the packaging cells. For example, if a viral vector is developed for delivery of a nuclease gene(s) to muscle tissue, a muscle-specific promoter can be used. Examples of muscle-specific promoters include C5-12 (Liu, et al. (2004) Hum Gene Ther. 15:783-92), the muscle-specific creatine kinase (MCK) promoter (Yuasa, et al. (2002) Gene Ther. 9:1576-88), or the smooth muscle 22 (SM22) promoter (Haase, et al. (2013) BMC Biotechnol. 13:49-54). Examples of CNS (neuron)-specific promoters include the NSE, Synapsin, and MeCP2 promoters (Lentz, et al. (2012) Neurobiol Dis. 48:179-88). Examples of liver-specific promoters include albumin promoters (such as Palb), human α1-antitrypsin (such as PalAT), and hemopexin (such as Phpx) (Kramer et al., (2003) Mol. Therapy 7:375-85), hybrid liver-specific promoter (hepatic locus control region from ApoE gene (ApoE-HCR) and a liver-specific alphal-antitrypsin promoter), human thyroxine binding globulin (TBG) promoter, and apolipoprotein A-II promoter. Examples of eye-specific promoters include opsin, and corneal epithelium-specific K12 promoters (Martin et al. (2002) Methods (28): 267-75) (Tong et al., (2007) J Gene Med, 9:956-66). These promoters, or other tissue-specific promoters known in the art, are not highly-active in HEK-293 cells and, thus, will not be expected to yield significant levels of nuclease gene expression in packaging cells when incorporated into viral vectors of the present invention. Similarly, the viral vectors of the present invention contemplate the use of other cell lines with the use of incompatible tissue specific promoters (i.e., the well-known HeLa cell line (human epithelial cell) and using the liver-specific hemopexin promoter). Other examples of tissue specific promoters include: synovial sarcomas PDZD4 (cerebellum), C6 (liver), ASB5 (muscle), PPPIR12B (heart), SLC5A12 (kidney), cholesterol regulation APOM (liver), ADPRHLI (heart), and monogenic malformation syndromes TP73L (muscle). (Jacox et al., (2010), PLOS One v.5(8):e12274).

Alternatively, the recombinant virus can be packaged in cells from a different species in which the nuclease is not likely to be expressed. For example, viral particles can be produced in microbial, insect, or plant cells using mammalian promoters, such as the well-known cytomegalovirus- or SV40 virus-early promoters, which are not active in the non-mammalian packaging cells. In a particular embodiment, viral particles are produced in insect cells using the baculovirus system as described by Gao, et al. (Gao et al. (2007), J. Biotechnol. 131(2):138-43). A nuclease under the control of a mammalian promoter is unlikely to be expressed in these cells (Airenne et al. (2013), Mol. Ther. 21(4): 739-49). Moreover, insect cells utilize different mRNA splicing motifs than mammalian cells. Thus, it is possible to incorporate a mammalian intron, such as the human growth hormone (HGH) intron or the SV40 large T antigen intron, into the coding sequence of a nuclease. Because these introns are not spliced efficiently from pre-mRNA transcripts in insect cells, insect cells will not express a functional nuclease and will package the full-length genome. In contrast, mammalian cells to which the resulting recombinant AAV particles are delivered will properly splice the pre-mRNA and will express functional nuclease protein. Haifeng Chen has reported the use of the HGH and SV40 large T antigen introns to attenuate expression of the toxic proteins barnase and diphtheria toxin fragment A in insect packaging cells, enabling the production of recombinant AAV vectors carrying these toxin genes (Chen, H (2012) Mol Ther Nucleic Acids. 1(11): e57).

The MTEM gene can be operably linked to an inducible promoter such that a small-molecule inducer is required for nuclease expression. Examples of inducible promoters include the Tet-On system (Clontech; Chen et al. (2015), BMC Biotechnol. 15(1):4)) and the RheoSwitch system (Intrexon; Sowa et al. (2011), Spine, 36(10): E623-8). Both systems, as well as similar systems known in the art, rely on ligand-inducible transcription factors (variants of the Tet Repressor and Ecdysone receptor, respectively) that activate transcription in response to a small-molecule activator (Doxycycline or Ecdysone, respectively). Practicing the current invention using such ligand-inducible transcription activators includes: 1) placing the nuclease gene under the control of a promoter that responds to the corresponding transcription factor, the nuclease gene having (a) binding site(s) for the transcription factor; and 2) including the gene encoding the transcription factor in the packaged viral genome. The latter step is necessary because the nuclease will not be expressed in the target cells or tissues following recombinant AAV delivery if the transcription activator is not also provided to the same cells. The transcription activator then induces nuclease gene expression only in cells or tissues that are treated with the cognate small-molecule activator. This approach is advantageous because it enables nuclease gene expression to be regulated in a spatio-temporal manner by selecting when and to which tissues the small-molecule inducer is delivered. However, the requirement to include the inducer in the viral genome, which has significantly limited carrying capacity, creates a drawback to this approach.

In another particular embodiment, recombinant AAVs are produced in a mammalian cell line that expresses a transcription repressor that prevents expression of the nuclease. Transcription repressors are known in the art and include the Tet-Repressor, the Lac-Repressor, the Cro repressor, and the Lambda-repressor. Many nuclear hormone receptors such as the ecdysone receptor also act as transcription repressors in the absence of their cognate hormone ligand. To practice the current invention, packaging cells are transfected/transduced with a vector encoding a transcription repressor and the nuclease gene in the viral genome (packaging vector) is operably linked to a promoter that is modified to comprise binding sites for the repressor such that the repressor silences the promoter. The gene encoding the transcription repressor can be placed in a variety of positions. It can be encoded on a separate vector; it can be incorporated into the packaging vector outside of the ITR sequences; it can be incorporated into the cap/rep vector or the adenoviral helper vector; or it can be stably integrated into the genome of the packaging cell such that it is expressed constitutively. Methods to modify common mammalian promoters to incorporate transcription repressor sites are known in the art. For example, Chang and Roninson modified the strong, constitutive CMV and RSV promoters to comprise operators for the Lac repressor and showed that gene expression from the modified promoters was greatly attenuated in cells expressing the repressor (Chang and Roninson (1996), Gene 183:137-42). The use of a non-human transcription repressor ensures that transcription of the nuclease gene will be repressed only in the packaging cells expressing the repressor and not in target cells or tissues transduced with the resulting recombinant AAV vector.

### 2.5 Methods for Producing Genetically-Modified Cells

The invention provides methods for producing genetically-modified cells, both in vitro and in vivo, using MTEMs comprising engineered meganucleases that bind and cleave recognition sequences found within mtDNA, such as human mtDNA. Cleavage at such recognition sequences can allow for NHEJ at the cleavage site, insertion of an exogenous sequence via homologous recombination, or degradation of the mtDNA.

The invention includes that an MTEM of the invention, or a nucleic acid encoding the MTEM, can be delivered (i.e., introduced) into cells, such as eukaryotic cells (e.g., human cells).

MTEMs of the invention can be delivered into a cell in the form of protein or, preferably, as a nucleic acid encoding the MTEM. Such nucleic acid can be DNA (e.g., circular or linearized plasmid DNA or PCR products) or RNA (e.g., mRNA). Accordingly, polynucleotides are provided herein that comprise a nucleic acid sequence encoding an MTEM disclosed herein. In specific embodiments, the polynucleotide is an mRNA. The polynucleotides encoding an MTEM disclosed herein can be operably linked to a promoter. In specific embodiments, expression cassettes are provided that comprise a promoter operably linked to a polynucleotide having a nucleic acid sequence encoding a MTEM disclosed herein.

For embodiments in which the MTEM coding sequence is delivered in DNA form, it should be operably linked to a promoter to facilitate transcription of the MTEM-encoding sequence. Mammalian promoters suitable for the invention include constitutive promoters such as the cytomegalovirus early (CMV) promoter (Thomsen et al. (1984), Proc Natl Acad Sci USA. 81(3):659-63), the SV40 early promoter (Benoist and Chambon (1981), Nature. 290(5804):304-10), a CAG promoter, an EF1 alpha promoter, or a UbC promoter, as well as inducible promoters such as the tetracycline-inducible promoter (Dingermann et al. (1992), Mol Cell Biol. 12(9):4038-45). An MTEM of the invention can also be operably linked to a synthetic promoter. Synthetic promoters can include, without limitation, the JeT promoter (WO 2002/012514). In specific embodiments, a nucleic acid sequence encoding an MTEM of the invention is operably linked to a tissue-specific promoter, such as a muscle cell-specific promoter, a skeletal muscle-specific promoter, a myotube-specific promoter, a muscle satellite cell-specific promoter, a neuron-specific promoter, an astrocyte-specific promoter, a microglia-specific promoter, an eye cell-specific promoter, a retinal cell-specific promoter, a retinal ganglion cell-specific promoter, a retinal pigmentary epithelium-specific promoter, a pancreatic cell-specific promoter, or a pancreatic beta cell-specific promoter.

In specific embodiments, a nucleic acid sequence encoding an MTEM is delivered on a recombinant DNA construct or expression cassette. For example, the recombinant DNA construct can comprise an expression cassette (i.e., “cassette”) comprising a promoter and a nucleic acid sequence encoding an engineered nuclease described herein.

In some embodiments, mRNA encoding the ME™ is delivered to a cell because this reduces the likelihood that the gene encoding the engineered nuclease will integrate into the genome of the cell.

Such mRNA encoding an ME™ can be produced using methods known in the art such as in vitro transcription. In some embodiments, the mRNA is 5′ capped using 7-methyl-guanosine, anti-reverse cap analogs (ARCA) (U.S. Pat. No. 7,074,596), CLEANCAP® analogs such as Cap 1 analogs (Trilink, San Diego, CA), or enzymatically capped using vaccinia capping enzyme or similar. In some embodiments, the mRNA may be polyadenylated. The mRNA may contain various 5′ and 3′ untranslated sequence elements to enhance expression the encoded MTEM and/or stability of the mRNA itself. Such elements can include, for example, posttranslational regulatory elements such as a woodchuck hepatitis virus posttranslational regulatory element. The mRNA may contain nucleoside analogs or naturally-occurring nucleosides, such as pseudouridine, 5-methylcytidine, N6-methyladenosine, 5-methyluridine, or 2-thiouridine. Additional nucleoside analogs include, for example, those described in U.S. Pat. No. 8,278,036.

Purified MTEMs can be delivered into cells to cleave mitochondrial DNA by a variety of different mechanisms known in the art, including those further detailed herein.

In another particular embodiment, a nucleic acid encoding an MTEM of the invention is introduced into the cell using a single-stranded DNA template. The single-stranded DNA can further comprise a 5′ and/or a 3′ AAV inverted terminal repeat (ITR) upstream and/or downstream of the sequence encoding the MTEM. The single-stranded DNA can further comprise a 5′ and/or a 3′ homology arm upstream and/or downstream of the sequence encoding the MTEM.

In another particular embodiment, genes encoding an MTEM of the invention is introduced into a cell using a linearized DNA template. Such linearized DNA templates can be produced by methods known in the art. For example, a plasmid DNA encoding a nuclease can be digested by one or more restriction enzymes such that the circular plasmid DNA is linearized prior to being introduced into a cell.

Purified MTEMs, or nucleic acids encoding MTEMs, can be delivered into cells to cleave mitochondrial DNA by a variety of different mechanisms known in the art, including those further detailed herein below.

In some embodiments, MTEMs, DNA/mRNA encoding MTEMs, or cells expressing MTEMs are formulated for systemic administration, or administration to target tissues, in a pharmaceutically acceptable carrier in accordance with known techniques. See, e.g., Remington, The Science And Practice of Pharmacy (21st ed., Philadelphia, Lippincott, Williams & Wilkins, 2005). In the manufacture of a pharmaceutical formulation according to the invention, proteins/RNA/mRNA/cells are typically admixed with a pharmaceutically acceptable carrier. The carrier must, of course, be acceptable in the sense of being compatible with any other ingredients in the formulation and must not be deleterious to the patient. The carrier can be a solid or a liquid, or both, and can be formulated with the compound as a unit-dose formulation.

In some embodiments, the MTEMs, or DNA/mRNA encoding the MTEMs, are coupled to a cell penetrating peptide or targeting ligand to facilitate cellular uptake. Examples of cell penetrating peptides known in the art include poly-arginine (Jearawiriyapaisarn, et al. (2008) Mol Ther. 16:1624-9), TAT peptide from the HIV virus (Hudecz et al. (2005), Med. Res. Rev. 25: 679-736), MPG (Simeoni, et al. (2003) Nucleic Acids Res. 31:2717-2724), Pep-1 (Deshayes et al. (2004) Biochemistry 43: 7698-7706, and HSV-1 VP-22 (Deshayes et al. (2005) Cell Mol Life Sci. 62:1839-49. In an alternative embodiment, MTEMs, or DNA/mRNA encoding MTEMs, are coupled covalently or non-covalently to an antibody that recognizes a specific cell-surface receptor expressed on target cells such that the MTEM protein/DNA/mRNA binds to and is internalized by the target cells. Alternatively, MTEM protein/DNA/mRNA can be coupled covalently or non-covalently to the natural ligand (or a portion of the natural ligand) for such a cell-surface receptor. (McCall, et al. (2014) Tissue Barriers. 2(4): e944449; Dinda, et al. (2013) Curr Pharm Biotechnol. 14:1264-74; Kang, et al. (2014) Curr Pharm Biotechnol. 15(3):220-30; Qian et al. (2014) Expert Opin Drug Metab Toxicol. 10(11): 1491-508).

In some embodiments, MTEMs, or DNA/mRNA encoding MTEMs, are encapsulated within biodegradable hydrogels. Hydrogels can provide sustained and tunable release of the therapeutic payload to the desired region of the target tissue without the need for frequent injections, and stimuli-responsive materials (e.g., temperature- and pH-responsive hydrogels) can be designed to release the payload in response to environmental or externally applied cues (Kang Derwent et al. (2008) Trans Am Ophthalmol Soc. 106:206-214).

In some embodiments, MTEMs, or DNA/mRNA encoding MTEMs, are coupled covalently or, preferably, non-covalently to a nanoparticle or encapsulated within such a nanoparticle using methods known in the art (Sharma, et al. (2014) Biomed Res Int. 2014). A nanoparticle is a nanoscale delivery system whose length scale is <1 μm, preferably <100 nm. Such nanoparticles may be designed using a core composed of metal, lipid, polymer, or biological macromolecule, and multiple copies of the nuclease proteins, mRNA, or DNA can be attached to or encapsulated with the nanoparticle core. This increases the copy number of the protein/mRNA/DNA that is delivered to each cell and, so, increases the intracellular expression of each MTEM to maximize the likelihood that the target recognition sequences will be cut. The surface of such nanoparticles may be further modified with polymers or lipids (e.g., chitosan, cationic polymers, or cationic lipids) to form a core-shell nanoparticle whose surface confers additional functionalities to enhance cellular delivery and uptake of the payload (Jian et al. (2012) Biomaterials. 33(30): 7621-30). Nanoparticles may additionally be advantageously coupled to targeting molecules to direct the nanoparticle to the appropriate cell type and/or increase the likelihood of cellular uptake. Examples of such targeting molecules include antibodies specific for cell-surface receptors and the natural ligands (or portions of the natural ligands) for cell surface receptors.

In some embodiments, the MTEMs or DNA/mRNA encoding the MTEMs are encapsulated within liposomes or complexed using cationic lipids (see, e.g., LIPOFECTAMINE™, Life Technologies Corp., Carlsbad, CA; Zuris et al. (2015) Nat Biotechnol. 33: 73-80; Mishra et al. (2011) J Drug Deliv. 2011:863734). The liposome and lipoplex formulations can protect the payload from degradation, enhance accumulation and retention at the target site, and facilitate cellular uptake and delivery efficiency through fusion with and/or disruption of the cellular membranes of the target cells.

In some embodiments, MTEMs, or DNA/mRNA encoding MTEMs, are encapsulated within polymeric scaffolds (e.g., PLGA) or complexed using cationic polymers (e.g., PEI, PLL) (Tamboli et al. (2011) Ther Deliv. 2(4): 523-536). Polymeric carriers can be designed to provide tunable drug release rates through control of polymer erosion and drug diffusion, and high drug encapsulation efficiencies can offer protection of the therapeutic payload until intracellular delivery to the desired target cell population.

In some embodiments, MTEMs, or DNA/mRNA encoding MTEMs, are combined with amphiphilic molecules that self-assemble into micelles (Tong et al. (2007) J Gene Med. 9(11): 956-66). Polymeric micelles may include a micellar shell formed with a hydrophilic polymer (e.g., polyethyleneglycol) that can prevent aggregation, mask charge interactions, and reduce nonspecific interactions.

In some embodiments, MTEMs, or DNA/mRNA encoding MTEMs, are formulated into an emulsion or a nanoemulsion (i.e., having an average particle diameter of <1 nm) for administration and/or delivery to the target cell. The term “emulsion” refers to, without limitation, any oil-in-water, water-in-oil, water-in-oil-in-water, or oil-in-water-in-oil dispersions or droplets, including lipid structures that can form as a result of hydrophobic forces that drive apolar residues (e.g., long hydrocarbon chains) away from water and polar head groups toward water, when a water immiscible phase is mixed with an aqueous phase. These other lipid structures include, but are not limited to, unilamellar, paucilamellar, and multilamellar lipid vesicles, micelles, and lamellar phases. Emulsions are composed of an aqueous phase and a lipophilic phase (typically containing an oil and an organic solvent). Emulsions also frequently contain one or more surfactants. Nanoemulsion formulations are well known, e.g., as described in U.S. Pat. Nos. 6,015,832, 6,506,803, 6,635,676, 6,559,189, and 7,767,216, each of which is incorporated herein by reference in its entirety.

In some embodiments, MTEMs, or DNA/mRNA encoding MTEMs, are covalently attached to, or non-covalently associated with, multifunctional polymer conjugates, DNA dendrimers, and polymeric dendrimers (Mastorakos et al. (2015) Nanoscale. 7(9): 3845-56; Cheng et al. (2008) J Pharm Sci. 97(1): 123-43). The dendrimer generation can control the payload capacity and size, and can provide a high payload capacity. Moreover, display of multiple surface groups can be leveraged to improve stability, reduce nonspecific interactions, and enhance cell-specific targeting and drug release.

In some embodiments, polynucleotides having nucleic acid sequences encoding an MTEM are introduced into a cell using a recombinant virus. Such recombinant viruses are known in the art and include recombinant retroviruses, recombinant lentiviruses, recombinant adenoviruses, and recombinant AAVs (reviewed in Vannucci, et al. (2013) New Microbiol. 36:1-22). Recombinant AAVs useful in the invention can have any capsid or serotype that allows for transduction of the virus into a target cell type and expression of the MTEM by the target cell. For example, in some embodiments, recombinant AAV has a serotype of AAV1, AAV2, AAV3, AAV3B, AAV4, AAV5, AAV6, AAV7, AAV8, AAV9, AAV10, AAV11, or AAVHSC. In some embodiments, the recombinant virus is injected directly into target tissues. In alternative embodiments, the recombinant virus is delivered systemically via the circulatory system. It is known in the art that different AAVs tend to localize to different tissues, and one could select an appropriate AAV capsid/serotype for preferential delivery to a particular tissue. Accordingly, in some embodiments, the AAV serotype is AAV2. In alternative embodiments, the AAV serotype is AAV6. In other embodiments, the AAV serotype is AAV8. In still other embodiments, the AAV serotype is AAV9. AAVs can also be self-complementary such that they do not require second-strand DNA synthesis in the host cell (McCarty, et al. (2001) Gene Ther. 8:1248-54). Polynucleotides delivered by recombinant AAV vectors can include left (5′) and right (3′) inverted terminal repeats.

In one embodiment, a recombinant virus used for delivery of a polynucleotide having nucleic acid sequences encoding an MTEM is a self-limiting recombinant virus. A self-limiting recombinant virus can have limited persistence time in a cell or organism due to the presence of a recognition sequence for an engineered meganuclease within the viral genome. Thus, a self-limiting recombinant virus can be engineered to provide coding for a promoter, an MTEM described herein, and a meganuclease recognition site within the ITRs. The self-limiting recombinant virus delivers the meganuclease gene to a cell, tissue, or organism, such that the MTEM is expressed and able to cut the genome of the cell at an endogenous recognition sequence within the genome. The delivered meganuclease will also find its target site within the self-limiting recombinant virus itself, and cut the viral genome at this target site. Once cut, the 5′ and 3′ ends of the viral genome will be exposed and degraded by exonucleases, thus killing the virus and ceasing production of the MTEM.

If the polynucleotides having nucleic acid sequences encoding an MTEM are delivered in DNA form (e.g. plasmid) and/or via a viral vector (e.g. AAV) they must be operably linked to a promoter. In some embodiments, this can be a viral promoter such as endogenous promoters from the viral vector (e.g. the LTR of a lentiviral vector) or constitutive or tissue-specific promoters described elsewhere herein. In a particular embodiment, polynucleotides having nucleic acid sequences encoding an MTEM are operably linked to a promoter that drives gene expression preferentially in the target cells, such as nerve cells, muscle cells, pancreatic cells, ocular cells, etc. In some embodiments, the target cell is a muscle cell, a skeletal muscle cell, a myotube cell, a muscle satellite cell, a neuron, an astrocyte, a microglia cell, an eye cell, a retinal cell, a retinal ganglion cell, a retinal pigmentary epithelium cell, a pancreatic cell, or a pancreatic beta cell, or wherein said population of target cells is a population of muscle cells, skeletal muscle cells, myotube cells, muscle satellite cells, neurons, astrocytes, microglia cells, eye cells, retinal cells, retinal ganglion cells, retinal pigmentary epithelium cells, pancreatic cells, or pancreatic beta cells or the population of target cells is a population of muscle cells, a skeletal muscle cells, a myotube cells, a muscle satellite cells, a neuron, an astrocyte, a microglia cells, an eye cells, a retinal cells, a retinal ganglion cells, a retinal pigmentary epithelium cells, a pancreatic cells, or a pancreatic beta cells, or wherein said population of target cells is a population of muscle cells, skeletal muscle cells, myotube cells, muscle satellite cells, neurons, astrocytes, microglia cells, eye cells, retinal cells, retinal ganglion cells, retinal pigmentary epithelium cells, pancreatic cells, or pancreatic beta cells.

In some embodiments, provided herein are methods for producing a genetically-modified eukaryotic cell or a genetically-modified eukaryotic cell population by introducing into the eukaryotic cell or eukaryotic cell population a polynucleotide of the present disclosure, such as a polynucleotide containing a nucleic acid sequence that encodes an engineered meganuclease described hereinabove. Upon expression in the eukaryotic cell or eukaryotic cell population, the engineered meganuclease localizes to the mitochondria, binds a recognition sequence in the mitochondrial genome, and generates a cleavage site. The cleavage site generated by the engineered meganuclease can be repaired by NHEJ repair pathway which may result in a nucleic acid insertion or deletion at the cleavage site. Additionally or alternatively, the cleavage site generated by the engineered meganuclease in the mitochondrial genome of the eukaryotic cell or eukaryotic cell population can be repaired by alternative nonhomologous end-joining (Alt-NHEJ) or microhomology-mediated end joining (MMEJ). The NHEJ or Alt-NHEJ/MMEJ can result in insertion and/or deletion of a nucleic acid at the cleavage site. In particular, the NHEJ or Alt-NHEJ/MMEJ can result in insertion and/or deletion of 1-1000 (e.g., 1-10, 10-100, 100-200, 200-300, 300-400, 400-500, 500-600, 600-700, 700-80, 800-900, or 900-1000) nucleotides, such as about 1, 5, 10, 15, 20, 25, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1000 nucleotides at the cleavage site. In some embodiments, mitochondrial genomes in a genetically-modified eukaryotic cell disclosed herein or a genetically-modified eukaryotic cell population disclosed herein can be degraded. In some such embodiments, the percentage of mitochondrial genomes comprising the recognition sequence is decreased by about 30%,35%, 40%, 45%, 50%, 55%, 60%, 65%, 70%, 75%, 80%, 85%, 90%, 95%, 96%, 97%, 98%, 99%, or 100%, or can be degraded by about can be degraded by about 30-40%, 40-50%, 50-60%, 60-70%, 70-80%, 80-90%, 90-100%, or more, compared to a control cell. In specific embodiments, mutant mitochondrial genomes comprising an MTEM recognition sequence of SEQ ID NO: 3 are degraded. By degrading mutant mitochondrial genomes having the MTEM recognition sequence, the overall ratio of wild-type mitochondrial genomes to mutant mitochondrial genomes will increase following administration or expression of an MTEM disclosed herein. In some embodiments, the ratio of wild-type to mutant mitochondrial genomes in a single genetically-modified eukaryotic cell disclosed herein or a population genetically-modified eukaryotic cells increases to about 5:95, about 10:90, about 15:85, about 20:80, about 25:75, about 30:70, about 35:65, about 40:60, about 45:55, about 50:50, about 55:45, about 60:40, about 65:35, about 70:30, about 75:25, about 80:20, about 85:15, about 90:10, about 95:5, about 20:1, about 50:1, about 100:1, about 150:1, about 200:1, about 250:1, about 300:1, about 350:1, about 400:1, about 450:1, about 500:1, about 550:1, about 600:1, about 650:1, about 700:1, about 750:1, about 800:1, about 850:1, about 900:1, about 950:1, about 1000:1, or more.

In particular embodiments, the percentage of wild-type genomes in a single genetically-modified eukaryotic cell disclosed herein or a population of genetically-modified eukaryotic cells disclosed herein, can increase as mutant mitochondrial genomes comprising SEQ ID NO: 3 are recognized, cleaved, and degraded by the MTEM. The percentage of wild-type mitochondrial genomes in a genetically-modified eukaryotic cell or genetically modified cell population disclosed herein can be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more, of the total mitochondrial genomes in the genetically-modified eukaryotic cell or genetically modified cell population when compared to a eukaryotic cell or eukaryotic cell population that does not express an MTEM disclosed herein. Likewise the percentage of mutant mitochondrial genomes comprising the recognition sequence of SEQ ID NO: 3 in the genetically-modified eukaryotic cell or genetically-modified cell population can decrease by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more when compared to a eukaryotic cell or eukaryotic cell population that does not express an MTEM disclosed herein.

In some embodiments, mitochondrial respiration in a genetically-modified eukaryotic cell or a genetically-modified eukaryotic cell population disclosed herein can increase by about 30%, about 35%, about 40%, about 45%, about 50%, about 55%, about 60%, about 65%, about 70%, about 75%, about 80%, about 85%, about 90%, about 95%, about 100%, or more when compared to a eukaryotic cell that does not express an MTEM disclosed herein. Mitochondrial respiration in a genetically-modified eukaryotic cell or a genetically-modified eukaryotic cell population disclosed herein can be increased by about 30-40%, 40-50%, 50-60%, 60-70%, 70-80%, 80-90%, 90-100%, or more when compared to a eukaryotic cell or eukaryotic cell population that does not express an MTEM disclosed herein.

In certain instances, the recognition sequence is within a region of the mitochondrial genome associated with a mitochondrial disorder.

Both normal and mutated mtDNA can exist in the same cell, a situation known as heteroplasmy. The number of defective mitochondria may be out-numbered by the number of normal mitochondria. Symptoms may not appear in any given generation until the mutation affects a significant proportion of mtDNA. The uneven distribution of normal and mutant mtDNA in different tissues can affect different organs in members of the same family. This can result in a variety of symptoms in affected family members.

In specific embodiments, the recognition sequence disclosed herein in the mitochondrial genome of the eukaryotic cell or eukaryotic cell population is positioned between nucleotides 8470 and 13,447 of the mitochondrial genome. In particular embodiments, the recognition sequence in the mitochondrial genome of the eukaryotic cell or eukaryotic cell population is positioned between nucleotides 8460 and 8580 or between nucleotides 13,437 and 13,457. In particular embodiments, the recognition sequence of SEQ ID NO: 3 is located only on mutant mitochondrial genomes. Upon expression in the genetically-modified eukaryotic cell or genetically-modified eukaryotic cell population, the MTEM can localize to the mitochondria, bind the recognition sequence in the mitochondrial genome, and generate a cleavage site. Thus, by targeting a recognition sequence only located on mutant genomes, the genomes can be cleaved and subsequently degraded. This specific degradation of mutant mitochondrial genomes can be used to help treat or alleviate the symptoms of the mitochondrial disorders (e.g., mitochondrial common deletion disorder or MELAS).

Accordingly, methods are provided herein for degrading mutant mitochondrial genomes in a target cell or a population of target cells by delivering to the target cell or population a polynucleotide comprising a nucleic acid sequence encoding an MTEM or an MTEM disclosed herein. In specific embodiments, the target cell or population of target cells comprise mutant mitochondrial genomes and the MTEM recognizes and cleaves the MTEM recognition sequence (e.g., SEQ ID NO: 3). The target cell or target cell population can be in a mammalian subject, such as a human subject. In some embodiments, the target cell is a muscle cell, a skeletal muscle cell, a myotube cell, a muscle satellite cell, a neuron, an astrocyte, a microglia cell, an eye cell, a retinal cell, a retinal ganglion cell, a retinal pigmentary epithelium cell, a pancreatic cell, or a pancreatic beta cell, or wherein said population of target cells is a population of muscle cells, skeletal muscle cells, myotube cells, muscle satellite cells, neurons, astrocytes, microglia cells, eye cells, retinal cells, retinal ganglion cells, retinal pigmentary epithelium cells, pancreatic cells, or pancreatic beta cells or the population of target cells is a population of muscle cells, a skeletal muscle cells, a myotube cells, a muscle satellite cells, a neuron, an astrocyte, a microglia cells, an eye cells, a retinal cells, a retinal ganglion cells, a retinal pigmentary epithelium cells, a pancreatic cells, or a pancreatic beta cells, or wherein said population of target cells is a population of muscle cells, skeletal muscle cells, myotube cells, muscle satellite cells, neurons, astrocytes, microglia cells, eye cells, retinal cells, retinal ganglion cells, retinal pigmentary epithelium cells, pancreatic cells, or pancreatic beta cells.

Methods of treating a condition associated with a mitochondrial disorder in a subject are disclosed herein. Such methods include administering to a subject a therapeutically-effective amount of a polynucleotide having a nucleic acid sequence encoding an MTEM, or a therapeutically-effective amount of an MTEM disclosed herein, wherein the MTEM produces a cleavage site of the recognition sequence of SEQ ID NO: 3 in mutant mitochondrial genomes having the mitochondrial deletion. The cleavage site produced in mutant mitochondrial genomes can lead to degradation of the mutant mitochondrial genomes. In specific embodiments, treating comprises reducing or alleviating at least one symptom of a condition associated with a mitochondrial disorder. For example, symptoms of the mtDNA common deletion include but are not limited to any symptom of myopathies, Alzheimer disease, Pearson's syndrome, photoaging of the skin, Kearns-Sayre syndrome (KSS), or chronic progressive external ophthalmoplegia (CPEO). Specifically, symptoms of the mtDNA common deletion can include pigmentary retinopathy, and PEO, cerebellar ataxia, impaired intellect (intellectual disability, dementia, or both), sensorineural hearing loss, ptosis, oropharyngeal and esophageal dysfunction, exercise intolerance, muscle weakness, cardiac conduction block, endocrinopathy, sideroblastic anemia and exocrine pancreas dysfunction, ptosis, impaired eye movements due to paralysis of the extraocular muscles (ophthalmoplegia), oropharyngeal weakness, or variably severe proximal limb weakness with exercise intolerance. In some embodiments, the condition is a condition of the bone marrow, the pancreas, muscle, skeletal muscle, central nervous system, the eye, or the ears. In some embodiments, the condition is Pearson Syndrome, Kearns-Sayre Syndrome (KSS), Progressive External Ophthalmoplegia (PEO), diabetes, or kidney dysfunction.

In some embodiments, a subject is administered a pharmaceutical composition disclosed herein at a dose of about 1×10¹⁰ gc/kg to about 1×10¹⁴ gc/kg (e.g., 1×10¹⁰ gc/kg, 1×10¹¹ gc/kg, 1×10¹² gc/kg, 1×10¹³ gc/kg, or 1×10¹⁴ gc/kg) of a nucleic acid encoding an MTEM. In some embodiments, a subject is administered a pharmaceutical composition at a dose of at least about 1×10¹⁰ gc/kg, at least about 1×10¹¹ gc/kg, at least about 1×10¹² gc/kg, at least about 1×10¹³ gc/kg, or at least about 1×10¹⁴ gc/kg of a nucleic acid encoding an MTEM. In some embodiments, a subject is administered a pharmaceutical composition at a dose of about 1×10¹⁰ gc/kg to about 1×10¹¹ gc/kg, about 1×10¹¹ gc/kg to about 1×10¹² gc/kg, about 1×10¹² gc/kg to about 1×10¹³ gc/kg, or about 1×10¹³ gc/kg to about 1×10¹⁴ gc/kg of a nucleic acid encoding an MTEM. In certain embodiments, a subject is administered a pharmaceutical composition at a dose of about 1×10¹² gc/kg to about 9×10¹³ gc/kg (e.g., about 1×10¹² gc/kg, about 2×10¹² gc/kg, about 3×10¹² gc/kg, about 4×10¹² gc/kg, about 5×10¹² gc/kg, about 6×10¹² gc/kg, about 7×10¹² gc/kg, about 8×10¹² gc/kg, about 9×10¹² gc/kg, about 1×10¹³ gc/kg, about 2×10¹³ gc/kg, about 3×10¹³ gc/kg, about 4×10¹³ gc/kg, about 5×10¹³ gc/kg, about 6×10¹³ gc/kg, about 7×10¹³ gc/kg, about 8×10¹³ gc/kg, or about 9×10¹³ gc/kg) of a nucleic acid encoding an MTEM.

In some embodiments, a subject is administered a lipid nanoparticle formulation at a dose of about 0.1 mg/kg to about 3 mg/kg of mRNA encoding an MTEM. In some embodiments, the subject is administered a lipid nanoparticle formulation at a dose of at least about 0.1 mg/kg, at least about 0.25 mg/kg, at least about 0.5 mg/kg, at least about 0.75 mg/kg, at least about 1.0 mg/kg, at least about 1.5 mg/kg, at least about 2.0 mg/kg, at least about 2.5 mg/kg, or at least about 3.0 mg/kg of mRNA encoding an MTEM. In some embodiments, the subject is administered a lipid nanoparticle formulation at a dose of within about 0.1 mg/kg to about 0.25 mg/kg, about 0.25 mg/kg to about 0.5 mg/kg, about 0.5 mg/kg to about 0.75 mg/kg, about 0.75 mg/kg to about 1.0 mg/kg, about 1.0 mg/kg to about 1.5 mg/kg, about 1.5 mg/kg to about 2.0 mg/kg, about 2.0 mg/kg to about 2.5 mg/kg, or about 2.5 mg/kg to about 3.0 mg/kg of mRNA encoding an MTEM.

The target tissue(s) for delivery of MTEMs of the invention, or nucleic acids encoding MTEMs of the invention, include without limitation, nerve tissue, muscle tissue, neuromuscular tissue, pancreatic tissue, and ocular/retinal tissue. In some embodiments, the target cell for delivery is a muscle cell, a skeletal muscle cell, a myotube cell, a muscle satellite cell, a neuron, an astrocyte, a microglia cell, an eye cell, a retinal cell, a retinal ganglion cell, a retinal pigmentary epithelium cell, a pancreatic cell, or a pancreatic beta cell, or wherein said population of target cells is a population of muscle cells, skeletal muscle cells, myotube cells, muscle satellite cells, neurons, astrocytes, microglia cells, eye cells, retinal cells, retinal ganglion cells, retinal pigmentary epithelium cells, pancreatic cells, or pancreatic beta cells or the population of target cells is a population of muscle cells, a skeletal muscle cells, a myotube cells, a muscle satellite cells, a neuron, an astrocyte, a microglia cells, an eye cells, a retinal cells, a retinal ganglion cells, a retinal pigmentary epithelium cells, a pancreatic cells, or a pancreatic beta cells, or wherein said population of target cells is a population of muscle cells, skeletal muscle cells, myotube cells, muscle satellite cells, neurons, astrocytes, microglia cells, eye cells, retinal cells, retinal ganglion cells, retinal pigmentary epithelium cells, pancreatic cells, or pancreatic beta cells.

In various embodiments of the methods described herein, the one or more MTEMs, polynucleotides encoding such MTEMs, or recombinant viruses comprising one or more polynucleotides encoding such MTEMs, as described herein, can be administered via any suitable route of administration known in the art. Accordingly, the one or more MTEMs, polynucleotides encoding such MTEMs, or recombinant viruses comprising one or more polynucleotides encoding such MTEMs, as described herein may be administered by an administration route comprising intravenous, intramuscular, intraperitoneal, subcutaneous, intrahepatic, transmucosal, transdermal, intraarterial, and sublingual. In some embodiments, MTEMs, or mRNA, or DNA vectors MTEMs, are supplied to target cells (e.g., nerve cells, muscle cells, pancreatic cells, ocular cells, etc.) via injection directly to the target tissue. In some embodiments, the eukaryotic cell is a stem cell, a CD34+HSC, a muscle cell, a skeletal muscle cell, a myotube cell, a muscle satellite cell, a neuron, an astrocyte, a microglia cell, an eye cell, a retinal cell, a retinal ganglion cell, a retinal pigmentary epithelium cell, a pancreatic cell, a pancreatic beta cell, a kidney cell, a bone marrow cell, or an ear hair cell. Other suitable routes of administration of the MTEMs, polynucleotides encoding such MTEMs, or recombinant viruses comprising one or more polynucleotides encoding such MTEMs may be readily determined by the treating physician as necessary.

In some embodiments, a therapeutically effective amount of MTEMs described herein is administered to a subject in need thereof. As appropriate, the dosage or dosing frequency of the MTEM may be adjusted over the course of the treatment, based on the judgment of the administering physician. Appropriate doses will depend, among other factors, on the specifics of any AAV chosen (e.g., serotype, etc.), on the route of administration, on the subject being treated (i.e., age, weight, sex, and general condition of the subject), and the mode of administration. Thus, the appropriate dosage may vary from patient to patient. An appropriate effective amount can be readily determined by one of skill in the art. Dosage treatment may be a single dose schedule or a multiple dose schedule. Moreover, the subject may be administered as many doses as appropriate. One of skill in the art can readily determine an appropriate number of doses. The dosage may need to be adjusted to take into consideration an alternative route of administration or balance the therapeutic benefit against any side effects.

Exogenous nucleic acid molecules of the invention may be introduced into a cell and/or delivered to a subject by any of the means previously discussed. In a particular embodiment, exogenous nucleic acid molecules are introduced by way of a recombinant virus, such as a lentivirus, retrovirus, adenovirus, or a recombinant AAV. Recombinant AAVs useful for introducing an exogenous nucleic acid molecule can have any serotype that allows for transduction of the virus into the cell and insertion of the exogenous nucleic acid molecule sequence into the cell genome, including those serotypes/capsids previously described herein. The recombinant AAVs can also be self-complementary such that they do not require second-strand DNA synthesis in the host cell. Exogenous nucleic acid molecules introduced using a recombinant AAV can be flanked by a 5′ (left) and 3′ (right) inverted terminal repeat.

In another particular embodiment, an exogenous nucleic acid molecule can be introduced into a cell using a single-stranded DNA template. The single-stranded DNA can comprise the exogenous nucleic acid molecule and, in particular embodiments, can comprise 5′ and 3′ homology arms to promote insertion of the nucleic acid sequence into the nuclease cleavage site by homologous recombination. The single-stranded DNA can further comprise a 5′ AAV inverted terminal repeat (ITR) sequence 5′ upstream of the 5′ homology arm, and a 3′ AAV ITR sequence 3′ downstream of the 3′ homology arm.

In another particular embodiment, polynucleotides comprising nucleic acid sequences encoding MTEMs of the invention and/or an exogenous nucleic acid molecule of the invention can be introduced into a cell by transfection with a linearized DNA template. A plasmid DNA encoding an MTEM and/or an exogenous nucleic acid molecule can, for example, be digested by one or more restriction enzymes such that the circular plasmid DNA is linearized prior to transfection into the cell.

When delivered to a cell, an exogenous nucleic acid of the invention can be operably linked to any promoter suitable for expression of the encoded polypeptide in the cell, including those mammalian promoters and inducible promoters previously discussed. An exogenous nucleic acid of the invention can also be operably linked to a synthetic promoter. Synthetic promoters can include, without limitation, the JeT promoter (WO 2002/012514).

### 2.6 Variants

The present invention encompasses variants of the polypeptide and polynucleotide sequences described herein.

As used herein, “variants” is intended to mean substantially similar sequences. A “variant” polypeptide is intended to mean a polypeptide derived from the “native” polypeptide by deletion or addition of one or more amino acids at one or more internal sites in the native protein and/or substitution of one or more amino acids at one or more sites in the native polypeptide. As used herein, a “native” polynucleotide or polypeptide comprises a parental sequence from which variants are derived. Variant polypeptides encompassed by the embodiments are biologically active. That is, they continue to possess the desired biological activity of the native protein; for example, the ability to bind and cleave recognition sequences found in mtDNA (e.g., human mtDNA), such as MIT 11-12 recognition sequence (SEQ ID NO: 3). Such variants may result, for example, from human manipulation. In some embodiments, biologically active variants of a native polypeptide of the embodiments (e.g., SEQ ID NO: 2 or 4), or biologically active variants of the recognition half-site binding subunits described herein, will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99%, sequence identity to the amino acid sequence of the native polypeptide, native subunit, native HVR1, or native HVR2 as determined by sequence alignment programs and parameters described elsewhere herein. A biologically active variant of a polypeptide or subunit of the embodiments may differ from that polypeptide or subunit by as few as about 1-40 amino acid residues, as few as about 1-20, as few as about 1-10, as few as about 5, as few as 4, 3, 2, or even 1 amino acid residue.

The polypeptides of the embodiments may be altered in various ways including amino acid substitutions, deletions, truncations, and insertions. Methods for such manipulations are generally known in the art. For example, amino acid sequence variants can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

In some embodiments, engineered meganucleases of the invention can comprise variants of the HVR1 and HVR2 regions disclosed herein. Parental HVR regions can comprise, for example, residues 24-79 or residues 215-270 of the exemplified engineered meganucleases. Thus, variant HVRs can comprise an amino acid sequence having at least 80%, at least 85%, at least 90%, at least 91%, at least 92%, at least 93%, at least 94%, at least 95%, at least 96%, at least 97%, at least 98%, at least 99%, or more, sequence identity to an amino acid sequence corresponding to residues 24-79 or residues 215-270 of the engineered meganucleases exemplified herein, such that the variant HVR regions maintain the biological activity of the engineered meganuclease (i.e., binding to and cleaving the recognition sequence). Further, in some embodiments of the invention, a variant HVR1 region or variant HVR2 region can comprise residues corresponding to the amino acid residues found at specific positions within the parental HVR. In this context, “corresponding to” means that an amino acid residue in the variant HVR is the same amino acid residue (i.e., a separate identical residue) present in the parental HVR sequence in the same relative position (i.e., in relation to the remaining amino acids in the parent sequence). By way of example, if a parental HVR sequence comprises a serine residue at position 26, a variant HVR that “comprises a residue corresponding to” residue 26 will also comprise a serine at a position that is relative (i.e., corresponding) to parental position 26.

In particular embodiments, engineered meganucleases of the invention comprise an HVR 1 that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more sequence identity to an amino acid sequence corresponding to residues 215-270 of SEQ ID NO: 2 or 4.

In certain embodiments, engineered meganucleases of the invention comprise an HVR2 that has 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more sequence identity to an amino acid sequence corresponding to residues 24-79 of SEQ ID NO: 2 or 4.

A substantial number of amino acid modifications to the DNA recognition domain of the wild-type I-CreI meganuclease have previously been identified (e.g., U.S. Pat. No. 8,021,867) which, singly or in combination, result in engineered meganucleases with specificities altered at individual bases within the DNA recognition sequence half-site, such that the resulting rationally-designed meganucleases have half-site specificities different from the wild-type enzyme. Table 1 provides potential substitutions that can be made in an engineered meganuclease monomer or subunit to enhance specificity based on the base present at each half-site position (−1 through −9) of a recognition half-site. Such substitutions are incorporated into variants of the meganucleases disclosed herein.

TABLE 1
Potential substitutions in engineered meganuclease variants

| | Favored Sense-Strand Base | | | | | | | | | | |
|---|---|---|---|---|---|---|---|---|---|---|---|
| Posn. | A | C | G | T | A/T | A/C | A/G | C/T | G/T | A/G/T | A/C/G/T |

| | | | | | | | | | | |
|---|---|---|---|---|---|---|---|---|---|---|
| −1 | Y75 | R70* | K70 | Q70* | | | | T46* | | G70 |
| | L75* | H75* | E70* | C70 | | | | | | A70 |
| | C75* | R75* | E75* | L70 | | | | | | S70 |
| | Y139* | H46* | E46* | Y75* | | | | | | G46* |
| | C46* | K46* | D46* | Q75* | | | | | | |
| | A46* | R46* | | H75* | | | | | | |
| | | | | H139 | | | | | | |
| | | | | Q46* | | | | | | |
| | | | | H46* | | | | | | |
| −2 | Q70 | E70 | H70 | Q44* | C44* | | | | | |
| | T44* | D70 | D44* | | | | | | | |
| | A44* | K44* | E44* | | | | | | | |
| | V44* | R44* | | | | | | | | |
| | I44* | | | | | | | | | |
| | L44* | | | | | | | | | |
| | N44* | | | | | | | | | |
| −3 | Q68 | E68 | R68 | M68 | | H68 | | Y68 | K68 | |
| | C24* | F68 | | C68 | | | | | | |
| | I24* | K24* | | L68 | | | | | | |
| | | R24* | | F68 | | | | | | |
| −4 | A26* | E77 | R77 | | | | | S77 | | S26* |
| | Q77 | K26* | E26* | | | | | Q26* | | |
| −5 | | E42 | R42 | | | K28* | C28* | | | M66 |
| | | | | | | | Q42 | | | K66 |
| −6 | Q40 | E40 | R40 | C40 | A40 | | | | | S40 |
| | C28* | R28* | | I40 | A79 | | | | | S28* |
| | | | | V40 | A28* | | | | | |
| | | | | C79 | H28* | | | | | |
| | | | | I79 | | | | | | |
| | | | | V79 | | | | | | |
| | | | | Q28* | | | | | | |
| −7 | N30* | E38 | K38 | I38 | | | C38 | | | H38 |
| | Q38 | K30* | R38 | L38 | | | | | | N38 |
| | | R30* | E30* | | | | | | | Q30* |
| −8 | F33 | E33 | F33 | L33 | | R32* | R33 | | | |
| | Y33 | D33 | H33 | V33 | | | | | | |
| | | | | I33 | | | | | | |
| | | | | F33 | | | | | | |
| | | | | C33 | | | | | | |
| −9 | | E32 | R32 | L32 | | | | D32 | | S32 |
| | | | K32 | V32 | | | | I32 | | N32 |
| | | | | A32 | | | | | | H32 |
| | | | | C32 | | | | | | Q32 |
| | | | | | | | | | | T32 |

Bold entries are wild-type contact residues and do not constitute “modifications” as used herein.
An asterisk indicates that the residue contacts the base on the antisense strand.

Certain modifications can be made in an engineered meganuclease monomer or subunit to modulate DNA-binding affinity and/or activity. For example, an engineered meganuclease monomer or subunit described herein can comprise a G, S, or A at a residue corresponding to position 19 of I-CreI SEQ ID NO: 1 (WO 2009001159), a Y, R, K, or D at a residue corresponding to position 66 of I-CreI, and/or an E, Q, or K at a residue corresponding to position 80 of I-CreI (U.S. Pat. No. 8,021,867).

For polynucleotides, a “variant” comprises a deletion and/or addition of one or more nucleotides at one or more sites within the native polynucleotide. One of skill in the art will recognize that variants of the nucleic acids of the embodiments will be constructed such that the open reading frame is maintained. For polynucleotides, conservative variants include those sequences that, because of the degeneracy of the genetic code, encode the amino acid sequence of one of the polypeptides of the embodiments. Variant polynucleotides include synthetically derived polynucleotides, such as those generated, for example, by using site-directed mutagenesis but which still encode an engineered meganuclease, or an exogenous nucleic acid molecule, or template nucleic acid of the embodiments. Generally, variants of a particular polynucleotide of the embodiments will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to that particular polynucleotide as determined by sequence alignment programs and parameters described elsewhere herein. Variants of a particular polynucleotide of the embodiments (i.e., the reference polynucleotide) can also be evaluated by comparison of the percent sequence identity between the polypeptide encoded by a variant polynucleotide and the polypeptide encoded by the reference polynucleotide.

The deletions, insertions, and substitutions of the protein sequences encompassed herein are not expected to produce radical changes in the characteristics of the polypeptide. However, when it is difficult to predict the exact effect of the substitution, deletion, or insertion in advance of doing so, one skilled in the art will appreciate that the effect will be evaluated by screening the polypeptide for its ability to preferentially bind and cleave recognition sequences found within human mtDNA, such as the MIT 11-12 recognition sequence (SEQ ID NO: 3).

Table 2 is a summary of the sequences disclosed herein.

TABLE 2
List of sequences

| SEQ ID NO: | SEQUENCE | DESCRIPTION |
|---|---|---|
| 1 | MNTKYNKEFLLYLAGFVDGDGSIIAQIK | Wild-type I-CreI sequence |
| | PNQSYKFKHQLSLAFQVTQKTQRRWFL | |
| | DKLVDEIGVGYVRDRGSVSDYILSEIKPL | |
| | HNFLTQLQPFLKLKQKQANLVLKIIWRL | |
| | PSAKESPDKFLEVCTWVDQIAALNDSKT | |
| | RKTTSETVRAVLDSLSEKKKSSP | |
| 2 | MNTKYNKEFLLYLAGFVDGDGSIIAQIK | MTEM meganuclease with |
| | PNQSYKFKHQLSLAFQVTQKTQRRWFL | wild-type I-CreI subunits |
| | DKLVDEIGVGYVRDRGSVSDYILSEIKPL | |
| | HNFLTQLQPFLKLKQKQANLVLKIIEQLP | |
| | SAKESPDKFLEVCTWVDQIAALNDSKTR | |
| | KTTSETVRAVLDSLPGSVGGLSPSQASSA | |
| | ASSASSSPGSGISEALRAGAGSGTGYNKE | |
| | FLLYLAGFVDGDGSIIAQIKPNQSYKFKH | |
| | QLSLAFQVTQKTQRRWFLDKLVDEIGVG | |
| | YVRDRGSVSDYILSEIKPLHNFLTQLQPF | |
| | LKLKQKQANLVLKIIEQLPSAKESPDKFL | |
| | EVCTWVDQIAALNDSKTRKTTSETVRAV | |
| | LDSLSEKKKSSP | |
| 3 | GATAAGGATTGTAAGACTTCATCC | MIT 11-12 recognition |
| | | sequence (sense) |
| 4 | MNTKYNKEFLLYLAGFVDGDGSIYATIM | MIT 11-12x.40 meganuclease |
| | PNQRAKFRHVLRLHFNVSQKTQRRWFL | amino acid sequence |
| | DKLVDEIGVGYVRDRGSVSDYVLSEIKP | |
| | LHNFLTQLQPFLKLKQKQANLVLKIIEQL | |
| | PSAKESPDKFLEVCTWVDQIAALNDSKT | |
| | RKTTSETVRAVLDSLPGSVGGLSPSQASS | |
| | AASSASSSPGSGISEALRAGAGSGTGYNK | |
| | EFLLYLAGFVDGDGSITATITPCQRVKFK | |
| | HYLQLRFNVSQKTQRRWFLDKLVDEIG | |
| | VGYVQDLGSVSEYRLSQIKPLHNFLTQL | |
| | QPFLKLKQKQANLVLKIIEQLPSAKESPD | |
| | KFLEVCTWVDQIAALNDSKTRKTTSETV | |
| | RAVLDSLSEKKKSSP | |
| 5 | ATGAATACAAAATATAATAAAGAGTTC | MIT 11-12x.40 meganuclease |
| | TTACTCTACTTAGCAGGGTTTGTAGAC | nucleic acid sequence |
| | GGTGACGGTTCCATCTATGCCACTATC | |
| | ATGCCTAATCAACGGGCTAAGTTCAGG | |
| | CACGTTCTGCGGCTCCATTTCAATGTCT | |
| | CTCAGAAGACACAGCGCCGTTGGTTCC | |
| | TCGACAAGCTGGTGGACGAGATCGGTG | |
| | TGGGTTACGTGCGGGACCGTGGCAGCG | |
| | TCTCCGATTACGTGCTGTCCGAGATCA | |
| | AGCCTTTGCATAATTTTTTAACACAACT | |
| | ACAACCTTTTCTAAAACTAAAACAAAA | |
| | ACAAGCAAATTTAGTTTTAAAAATTAT | |
| | TGAACAACTTCCGTCAGCAAAAGAATC | |
| | CCCGGACAAATTCTTAGAAGTTTGTAC | |
| | ATGGGTGGATCAAATTGCAGCTCTGAA | |
| | TGATTCGAAGACGCGTAAAACAACTTC | |
| | TGAAACCGTTCGTGCTGTGCTAGACAG | |
| | TTTACCAGGATCCGTGGGAGGTCTATC | |
| | GCCATCTCAGGCATCCAGCGCCGCATC | |
| | CTCGGCTTCCTCAAGCCCGGGTTCAGG | |
| | GATCTCCGAAGCACTCAGAGCTGGAGC | |
| | AGGTTCCGGCACTGGATACAACAAGG | |
| | AATTCCTGCTCTACCTGGCGGGCTTCG | |
| | TCGACGGGGACGGCTCCATCACGGCCA | |
| | CTATCACGCCTTGTCAACGTGTGAAGT | |
| | TCAAGCACTATCTGCAGCTCCGTTTCA | |
| | ATGTCTCTCAGAAGACACAGCGCCGTT | |
| | GGTTCCTCGACAAGCTGGTGGACGAGA | |
| | TCGGTGTGGGTTACGTGCAGGACCTGG | |
| | GCAGCGTCTCCGAGTACCGTCTGTCCC | |
| | AGATCAAGCCTCTGCACAACTTCCTGA | |
| | CCCAGCTCCAGCCCTTCCTGAAGCTCA | |
| | AGCAGAAGCAGGCCAACCTCGTGCTG | |
| | AAGATCATCGAGCAGCTGCCCTCCGCC | |
| | AAGGAATCCCCGGACAAGTTCCTGGAG | |
| | GTGTGCACCTGGGTGGACCAGATCGCC | |
| | GCTCTGAACGACTCCAAGACCCGCAAG | |
| | ACCACTTCCGAAACCGTCCGCGCCGTT | |
| | CTAGACAGTCTCTCCGAGAAGAAGAA | |
| | GTCGTCCCCC | |
| 6 | MSVLTPLLLRGLTGSARRLPVPRAKIHSL | COX VIII MTP |
| | PPEGKL | |
| 7 | MASTRVLASRLASQMAASAKVARPAVR | SU9 MTP |
| | VAQVSKRTIQTGSPLQTLKRTQMTSIVN | |
| | ATTRQAFQ | |
| 8 | MSVLTPLLLRGLTGSARRLPVPRAKIHSL | COX VIII-SU9 MTP |
| | PPEGKLMASTRVLASRLASQMAASAKV | |
| | ARPAVRVAQVSKRTIQTGSPLQTLKRTQ | |
| | MTSIVNATTRQAFQ | |
| 9 | VDEMTKKFGTLTIHDTEK | MVMp NS2 NES sequence |
| 10 | LGAGLGALGL | NES sequence |
| 11 | CTATTCCTAACATTCTGAAGTAGG | MIT 11-12 recognition |
| | | sequence (antisense) |
| 12 | CCACCCTAGCTATCATAAGCACA | m.5024C > T forward primer |
| 13 | AAGCAATTGATTTGCATTCAATAGATG | m.5024C > T reverse primer |
| | TAGGATGAAGTCCTGCA | |
| 14 | GCC TGA CCC ATA GCC ATA AT | ND1 forward primer |
| 15 | CGG CTG CGT ATT CTA CGT TA | ND1 reverse primer |
| 16 | TCT CAA CCC TAG CAG AAA CAA CCG | ND1 probe |
| | G | |
| 17 | CCC ATG ACT ACC ATC AGC AAT AG | ND5 forward primer |
| 18 | TGG AAT CGG ACC AGT AGG AA | ND5 reverse primer |
| 19 | AGT GCT GAA CTG GTG TAG GGC | ND5 probe |
| 20 | GCC GCT AGA GGT GAA ATT CT | 18s forward primer |
| 21 | TCG GAA CTA CGA CGG TAT CT | 18s reverse primer |
| 22 | AAG ACG GAC CAG AGC GAA AGC AT | 18s probe |
| 23 | GACTTCATCCTACATCTATTG | biotinylated probe to detect |
| | | mitochondrial tRNA^{Ala} |
| 24 | GTAAGGATAGTAAGTCTTCCAA | C57BL6J chromosome 2 site |
| | | 1 |
| 25 | TCAAGGATAGCACGTCTCCAAC | C57BL6J chromosome 2 site |
| | | 2 |
| 26 | TTAAGGATGGTAAGACAGTATC | C57BL6J chromosome 5 site |
| | | 3 |
| 27 | CTAAGGTTAGTAAGTATTCAAC | C57BL6J chromosome 6 site |
| | | 4 |
| 28 | TTCAGGATGGCACGTCTTCATC | C57BL6J chromosome 2 site |
| | | 5 |
| 29 | AGCCCCGGGTACTCCTTGTT | probe for APC 11-12 binding |
| | | site |
| 30 | TTCCTTGCAGGAACAGAG | forward primer for APC 11-12 |
| | | binding site |
| 31 | CTGCTTGACCACCCATT | reverse primer for APC 11-12 |
| | | binding site |
| 32 | CCAGCAGGCCAGGTACACC | probe for reference binding |
| | | site |
| 33 | ACCGCCAAGGATGCAC | forward primer for reference |
| | | binding site |
| 34 | GCGGGTGGGAATGGAG | reverse primer for reference |
| | | binding site |

### EXAMPLES

This disclosure is further illustrated by the following examples, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1. Mitochondria-Targeting Engineered Meganuclease (MTEM) Construct Design

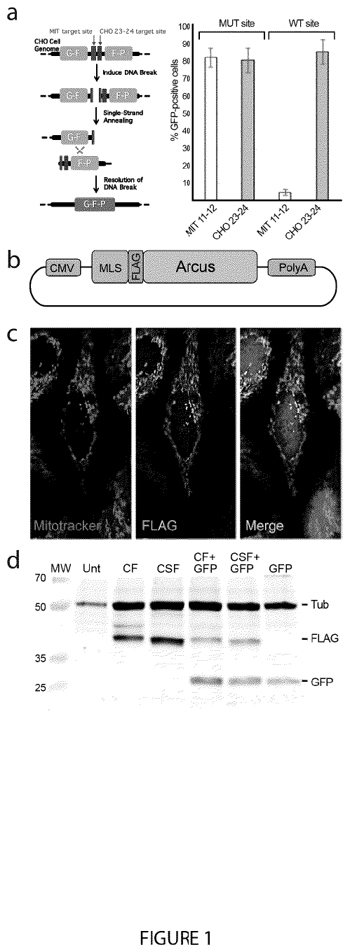

In silico predictions and directed evolution methods were used to engineer I-CreI in order to recognize the m.5024C>T point mutation (see, for example, U.S. Pat. No. 8,445,251 and U.S. Patent Publication No. 20200109383, each of which is herein incorporated by reference in its entirety). A peptide-linker was used to fuse both homodimers into a monomeric structure. The specificity of the mitochondrial-targeted engineered meganucleases (MTEM) was initially confirmed in Chinese Hamster Ovary (CHO) cells using an engineered split-GFP assay as described in FIG. 1A.

An engineered meganuclease recognizing the mutated (m.5024C>T) mouse mtDNA sequence (ATAAGGATTGTAAGACTTCATC, SEQ ID NO: 3) was produced. Analysis of the engineered meganuclease using a green fluorescent protein (GFP)-based double strand break (DSB) recombination assay in CHO cells showed that the specific engineered meganuclease (referred to herein as MIT 11-12) yielded ˜80% GFP+ cells when tested against the intended (mutant) target sites but <5% GFP+ cells when tested against the wild-type target sites (FIG. 1A).

For ex vivo expression, two variants were designed (CF and CSF) which differed by the mitochondrial localization sequence (MLS). A Cox 8 [construct CF, as described in Candas et al. (2016) J Vis Exp 108, p. 53417], or a Cox8/Su9 [construct CSF, as described in Bacman (2018) Nat Med. 24(11): p. 1696-1700 and Pereira et al. (2018) EMBO Mol Med. 10(9)] was constructed with the mitochondrial transit peptide (MTP) at the 5′ end of the construct. A FLAG tag was also added between the MTP and the engineered meganuclease (FIG. 1B).

HeLa cells were plated onto coverslips and transfected with MTEM for 24 h. Cells were incubated for 1 h at 37° ° C. with 200 nM MITOTRACKER™ Red CMXRos (M7512, Invitrogen), and protected from light. The cells were fixed with 2% paraformaldehyde (PFA) for 15 min at room temperature (RT). Cells were permeabilized with 0.2% Triton X-100 in phosphate saline buffer (PBS) for 2 min at RT. A 3% BSA/PBS solution was used as a blocking agent for 1 h at RT. The primary antibody against FLAG (F3165, Sigma-Aldrich) at a 1:200 concentration in 2% BSA/PBS was incubated for 1 h at RT. After washing, cells were incubated with goat anti-rabbit IgG (A-11008, Invitrogen) secondary antibody at a 1:200 concentration in 3% BSA/PBS for 1 hr at RT, and protected from light. Coverslips were then washed with PBS and mounted onto slides using a DAPI-containing mounting medium (EVERBRITE mounting medium, Biotium). Images were captured using a Zeiss LSM510 confocal microscope.

Transient transfection in HeLa cells showed that both MTEM constructs synthesized proteins that localized to mitochondria (FIG. 1C, which shows a representative cell for the CSF construct). A Western blot confirmed that both CF and CSF constructs expressed the expected sized proteins after transfection of HEK293T cells (FIG. 1D). The pattern in the Western blot showed no spurious bands in the CSF-transfected cells, therefore, the CSF variant was selected for subsequent experiments.

### Example 2. Animal Model and Fibroblasts

A heteroplasmic mouse model carrying an m.5024C>T point mutation in the tRNA^{Ala} gene was used to evaluate the efficacy of the MTEM constructs. This mouse model has been previously characterized (Bacman et al. (2018) Nat Med. 24(11): p. 1696-1700; Kauppila et al. (2016) Cell Rep. 16(11): p. 2980-2990; and Gammage et al (2018) Nat Med. 24(11): p. 1691-1695) and presents with a mild cardiomyopathy at 2 years of age, as well as reduced mt-tRNA^{Ala} levels when mutation levels are greater than 50% in the tissue. Mouse embryonic fibroblasts (MEF) derived from this mouse were characterized for the levels of mutant mtDNA and immortalized with the E6-E7 gene of the human papilloma virus (Bacman 2018, supra). Wild type animals and wild type lung fibroblast cells used as controls were derived from mice with C57BL/6J background. To produce an MEF line with high levels of mutation, a mitoTALEN targeting WT mtDNA was designed. MEFs were transfected with the mitoTALEN, sorted, and clones were grown and tested for high levels of mutant mtDNA (Bacman 2018, supra).

Heteroplasmic MEFs were transfected using GENJET™ DNA In Vitro Transfection Reagent (Ver. II; SL100489, SignaGen Laboratories) using the manufacturer's protocols. Cells plated in a T75 flask at 80% confluence were transfected with 30 μg plasmid, in a 2:1 ratio of MTEM CF or CSF plasmid (20 μg) to green fluorescent protein (GFP) plasmid (10 μg). Twenty-four hours after transfection, sorting was performed using FACS Aria IIU, gating on single cell fluorescence using a 488 nm laser and 505LP, 530/30 filter set for GFP expression. Cells were sorted based on populations showing no GFP expression, shown herein as black, and populations showing GFP expression, shown herein as green. Untransfected cells were used as controls.

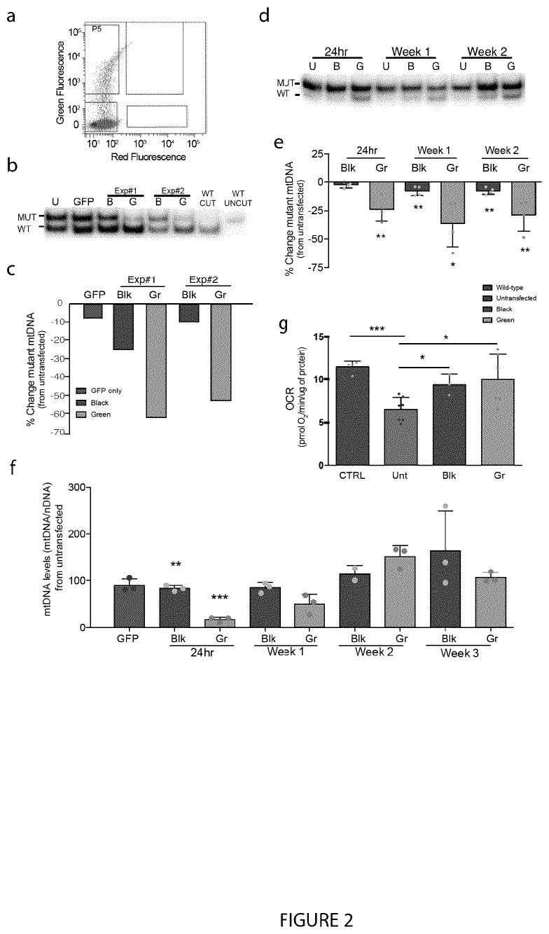

Transfection efficiency was relatively low, therefore, a MTEM expressing plasmid was co-transfected with a plasmid expressing GFP (2 MTEM: 1 GFP ratio). GFP-positive cells comprised 11-20% of the total cell population after transfection. Co-transfected cells were FACS sorted 24 hours later into non-transfected (black) and transfected (green) populations (FIG. 2A). PCR/RFLP was used to determine mtDNA heteroplasmy changes in the non-transfected and transfected cell populations of two independent experiments (FIG. 2B). Quantification showed that there was a large shift (50-60%) in heteroplasmy in the transfected population when compared to the non-transfected population. Cells transfected with the GFP plasmid only did not show changes in heteroplasmy. There was a small shift in the non-transfected cell populations (10-20%) that may be due to a population not having incorporated GFP co-transfectant plasmid, but likely did incorporate the MTEM plasmid (FIG. 2C).

To determine the biological significance of heteroplasmic change, an MEF cell line that harbored high levels (90%) of mutant mtDNA was used (Bacman, 2018, supra). Cells were co-transfected with plasmids expressing MTEM and GFP and sorted as described infra. MtDNA was analyzed 1, 7, 14, and 21 days after transfection by PCR/RFLP. GFP-positive cells comprised 11-20% of the total cell population after transfection. There was a significant shift in heteroplasmy (approximately 25%) in the transfected cells at 24 hours post-transfection that was maintained over a two-week period (FIG. 2D and FIG. 2E). There was a depletion of total mtDNA levels in the transfected populations 24 hours after transfection, which then returned to baseline after 21 days (FIG. 2F). The non-transfected population had a very mild decrease, possibly due to some cells receiving the MTEM, as previously discussed.

Cells grown for 3 weeks were analyzed for their Oxygen Consumption Rate (OCR). OCR was measured using a Seahorse XFp Extracellular Flux Analyzer (Seahorse Bioscience). The day prior to the assay, cells were seeded at a density of 20,000 cells/well in wells B-G (wells A and H contained media only). The XFp sensor cartridge was calibrated with calibration buffer overnight at 37ºC. The following day, cell culture medium was replaced with low-buffered Seahorse medium supplemented with 10 mM glucose, 1 mM pyruvate, and 2 mM glutamine, and incubated for at least 1 h at 37° C. Measurements of endogenous respiration were taken following each addition of luM oligomycin, 0.5 μM FCCP, and 1 μM rotenone plus antimycin A. Results were normalized to μg protein per well after the Seahorse run, and protein was quantified using DC protein assay (Pereira 2018, supra).

Untransfected cells had impaired respiration compared to wild-type controls. Transfected cell populations had significantly improved OCR (FIG. 2G). A slight improvement in OCR in the non-transfected cells was also observed (FIG. 2G), likely due to the mild change in heteroplasmy as discussed elsewhere herein.

### Example 3. Expression of MTEM in Mice

DNA encoding the MTEM was cloned into an AAV2/9 plasmid and sent to the University of Iowa Viral Core Facility, which produced virus with 1.6×10¹³ vg/ml titer for AAV-MTEM, and 5.9×10¹³ vg/ml of AAV9-GFP. Both juvenile (2.5 weeks of age) and adult mice (6 weeks of age) were injected retro-orbitally as described [Bacman 2018 and Yardeni et al. (2011) Lab Anim (NY) 40(5): p. 155-60]. Juvenile mice received 6.67×10¹³ vgs/kg of AAV9-MTEM or 6.15×10¹³ vgs/kg of AAV9-GFP into the left retro-orbital sinus. Adults received 4.0×10¹³ vgs/kg of AAV9-MTEM or 3.69×10¹³ vgs/kg AAV9-GFP into the left retro-orbital sinus. Control animals were injected with similar titers of AAV9-GFP, respective of age-matched experimental animals. Toe biopsies were collected at 6 days of age to determine base heteroplasmy levels. At 6, 12, and 24 weeks post-injection (PI), mice were anesthetized with Ketamine and Xylazine and perfused with PBS. Heart, tibialis anterior, quadriceps, gastrocnemius, kidney, liver, and spleen were collected. Samples were flash frozen in liquid nitrogen and then stored at −80° C. until further use.

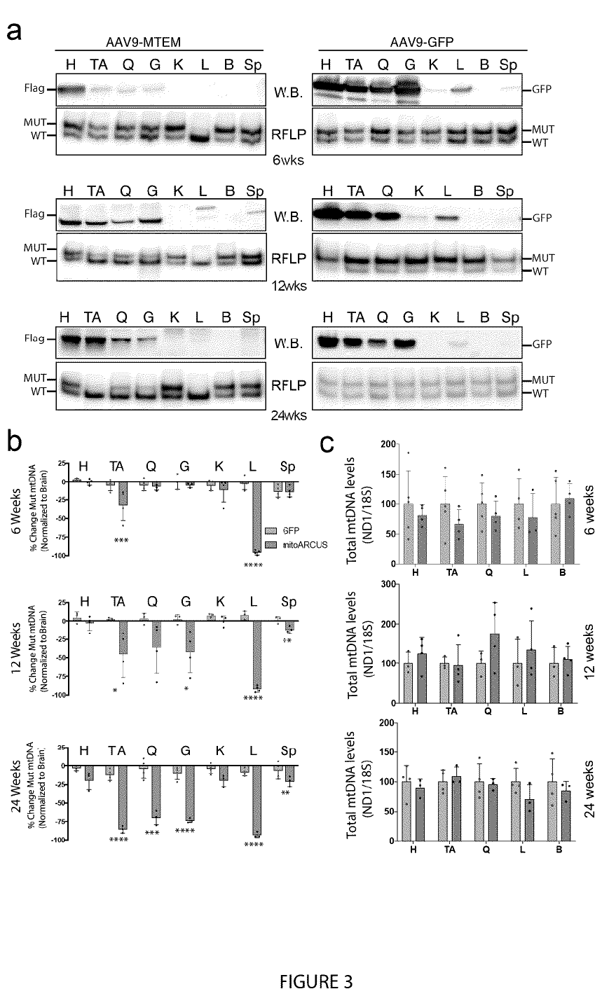

AAV9-MTEM injected animals showed consistent expression of MTEM in heart, skeletal muscles, and, in some animals, liver at all time points: 6, 12, and 24 weeks post-injection (PI), as shown by Western blot analysis using the anti-Flag antibody against the construct (FIG. 3A). AAV9-GFP injected animals showed GFP expression in the same tissues, although with higher expression in liver at 6 and 12 weeks post-injection (FIG. 3A). Corresponding RFLP analysis showed a significant decrease in mutant mtDNA in liver and tibialis anterior samples at 6 weeks PI in AAV9-MTEM injected animals (FIG. 3A). Heteroplasmy shifts became greater over time, and at 24 weeks PI, there was a significant decrease in mutant mtDNA in heart, skeletal muscles, kidney, liver, and spleen (FIG. 3B). AAV9-GFP injected controls had similar levels of mutation across all tissues after injection (FIG. 3A-3B). Brain tissue was used as a negative control to normalize changes in heteroplasmy since no expression was observed in brain after injection of either AAV9-MTEM or AAV9-GFP (FIG. 3A). There was no significant depletion of mtDNA levels seen in any of the analyzed tissues at 6, 12, or 24 weeks PI (FIG. 3C), suggesting minimal or no non-specific mtDNA effects. Mice weights did not differ between MTEM treated and control animals at all time points.

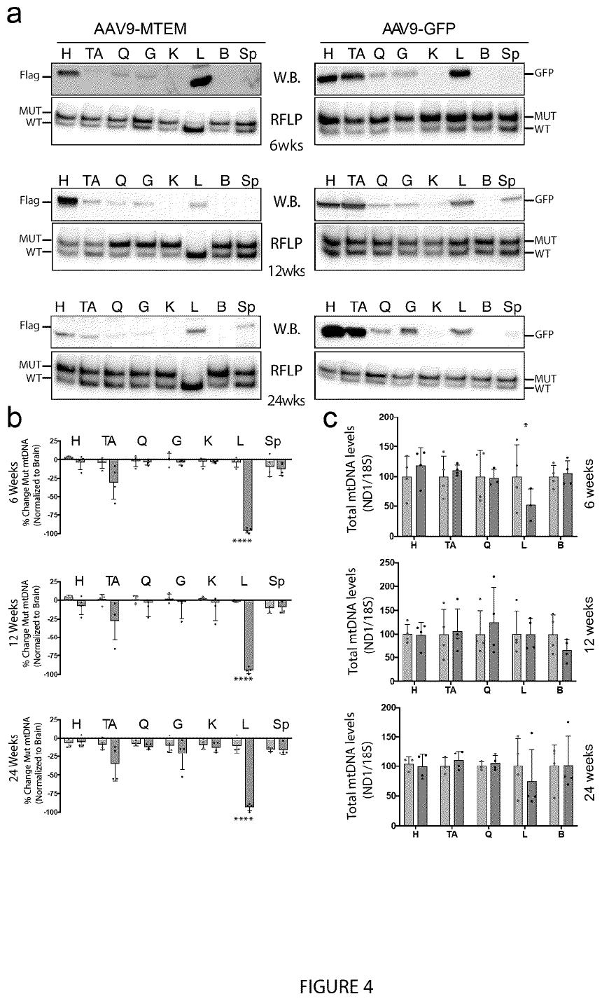

Heteroplasmic m.5024C>T mice were also injected systemically with AAV9—MTEM at 6 weeks of age, an age that is less permissive to AAV-mediated expression compared to 2.5 weeks. Still, strong expression was observed in heart and liver, with weaker expression in skeletal muscles (FIG. 4A). AAV9-GFP injected mice showed strong expression in heart, tibialis anterior, and liver, with weaker expression in quadriceps and gastrocnemius (FIG. 4A). RFLP analysis showed an essentially complete elimination of mutant mtDNA in liver as early as 6 weeks after injection in MTEM treated animals, that persisted over time (FIG. 4B). Some skeletal muscles showed a trend in decreasing mutant mtDNA (tibialis anterior, gastrocnemius at 24 weeks PI), but results did not reach significance (FIG. 4B). AAV9-GFP injected animals showed no change in the levels of heteroplasmy across all tissues (FIG. 4A and FIG. 4B). Brain was used to normalize the changes. Total mtDNA levels were slightly decreased in liver 6 weeks PI of the AAV9—MTEM treated animals, but not after 12 or 24 weeks PI (FIG. 4C).

MTEM was not detected at 6, 12, or 24 weeks PI, In the animals injected at 2.5 weeks, and sacrificed at 5 and 10 days PI, MTEM expression was detected solely in the liver. However, GFP expression was visible in heart, skeletal muscles, and liver, possibly because of higher expression of this construct. Heteroplasmy changes at 5 and 10 days PI were only observed in liver. There was an average of 22% reduction in mutant mtDNA at 5 days PI, which increased to 55% at 10 days PI. To determine if apoptosis played a role in these changes, expression of PCNA (marker of liver regeneration), Caspase3, and cleaved-Caspase3 was analyzed in liver samples. No differences between MTEM treated animals and control animals were observed at either time point. Similar levels of uncleaved caspase3 were observed in treated and control animals, but not cleaved-caspase3. In addition, depletion of total mtDNA levels at 5 or 10 days PI was not observed in liver or tibialis anterior. Furthermore, liver H&E staining did not show any morphological differences between AAV9-MTEM injected, AAV9-GFP injected, and non-injected controls.

The results showed that the MTEM localized exclusively to mitochondria, however, nuclear off-target editing was evaluated. To do so, targeted amplicon sequencing was performed on a selection of the sites that were identified from an in vitro, genome-wide, unbiased off-targeting assay based on GUIDESeq. DNA from the tibialis anterior (TA) and liver (L) tissues from the young mice at 24 weeks PI were evaluated by targeted amplicon sequencing. This method of analysis detects any genetic variation within the amplicon, such as an insertion/deletion (indel). No indels were detected at any of the sites analyzed for any of the animals.

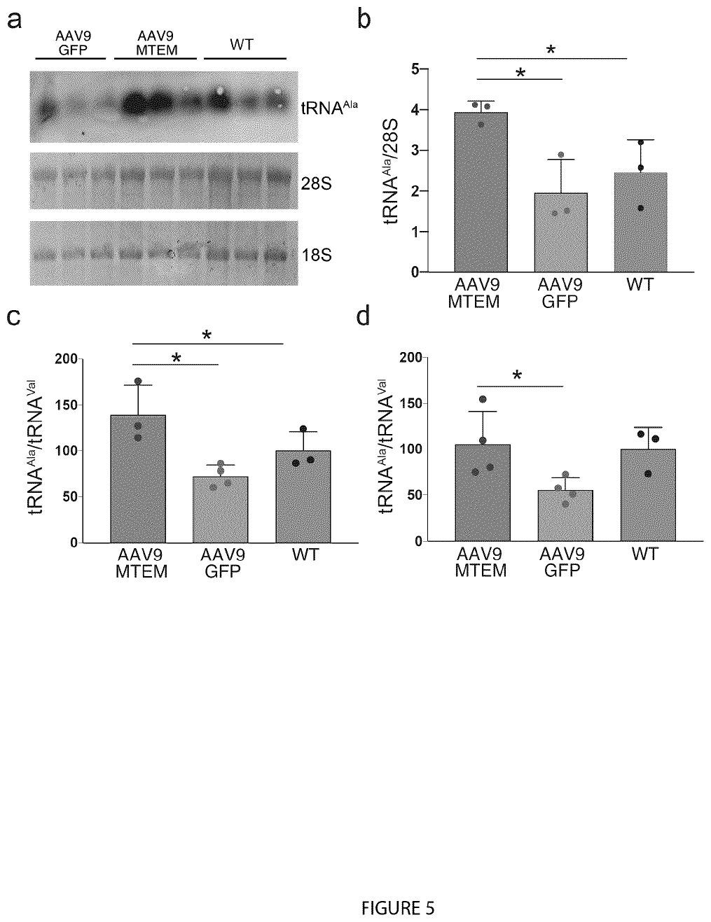

Mt-tRNA^{Ala} levels are decreased in tissues with high levels of mutant mtDNA in the m.5024C>T tRNA^{Ala} mice (Bacman 2018 and Kauppila et al. (2016) Cell Rep. 16(11): p. 2980-2990]. The mt-tRNA^{Ala} levels in liver of juvenile mice at 24 weeks PI was determined by Northern blot. Results showed an increased amount of mt-tRNA^{Ala} in AAV9-MTEM injected animals compared to AAV9-GFP injected controls when normalized to 28S rRNA (FIG. 5A and FIG. 5B). Quantitative PCR was used to determine the ratio of mt-tRNA^{Ala} to mt-tRNA^{Val}. These results confirmed the Northern blot results (FIG. 5C and FIG. 5D). By qPCR, it was shown that AAV9-MTEM treated juvenile animals had significantly higher levels of mt-tRNA^{Ala} compared to controls (AAV9-GFP), and even wild-type animals (FIG. 5C). Adult animals injected with AAV9-MTEM also had increased mt-tRNA^{Ala} levels in liver compared to controls (AAV9-GFP) (FIG. 5D).

### Example 4: Nuclease Activity with Mitochondrial Localization and Addition of Nuclear Export Sequence

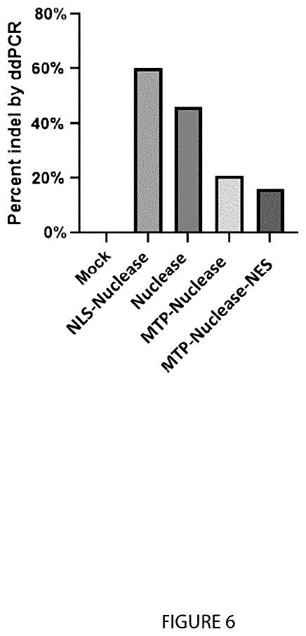

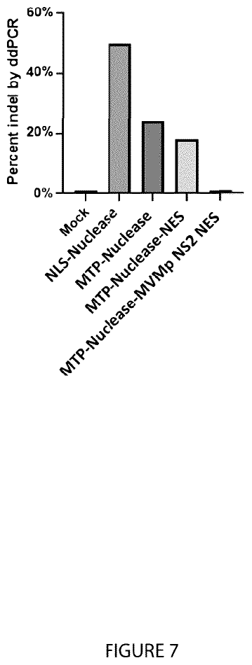

The purpose of this experiment was to determine if the addition of a nuclear export signal (NES) onto engineered meganucleases would eliminate nuclear indels. The NES used in FIG. 6 was rationally designed based on data from Kosugi et al 2008 Traffic 12:2053-62. The NES amino acid sequence, fused to the C-terminus of the engineered meganuclease, was: LGAGLGALGL (SEQ ID NO: 10). The NES used in FIG. 7 was taken from Minczuk et al 2006 Proc Natl Acad Sci USA 103(52):19689-19694. The NES amino acid sequence, fused to the C-terminus of the engineered meganuclease, was: VDEMTKKFGTLTIHDTEK (SEQ ID NO: 9).

The engineered meganuclease used in FIGS. 6 and 7 was APC 11-12L.330, which has a nuclear target site. 6e5 MRC-5 cells were nucleofected with an equal number of engineered meganuclease mRNA copies using the Lonza 4D-Nucleofector™ (SE buffer, condition CM-150). Four meganuclease constructs were compared in FIG. 6: one with an NLS, one with no targeting sequence, one with a mitochondrial transit peptide (MTP), and one with an MTP and NES. Four engineered meganuclease constructs were compared in FIG. 7: one with an NLS, one with an MTP, one with an MTP and NES, and one with an MTP and MVMp NS2 NES. The NLS and MTP were both fused to the N-terminus of their respective proteins, and the NES was fused to the C-terminus. Since these different constructs yield different length mRNAs, the mRNA copy number was kept consistent across transfections (5.8e11 copies for the data in FIG. 6, 2.88e11 copies for the data in FIG. 7). Cells were collected at two days post-nucleofection for gDNA extraction and evaluated for transfection efficiency using a Beckman Coulter CytoFlex S cytometer. Transfection efficiency exceeded 95%. gDNA was isolated using the Macherey Nagel NucleoSpin Blood QuickPure kit.

Digital droplet PCR (ddPCR) was utilized to determine indel frequency at the APC 11-12 binding site using P1, F1, and R1 (SEQ ID NO: 29, 30 and 31, respectively) to generate an amplicon surrounding the binding site, as well as P2, F2, R2 (SEQ ID NO: 32, 33 and 34, respectively) to generate a reference amplicon that acts as a genomic counter. The ratio of the two amplicons should be equal in an un-treated population and drop relative to indel formation at the binding site in treated samples. Amplifications were multiplexed in a 24 μL reaction containing 1×ddPCR Supermix for Probes (no dUTP, BioRad), 250 nM of each probe, 900 nM of each primer, 20 U/μL Hind-III HF (NEB), and 120 ng cellular gDNA. Droplets were generated using a QX100 droplet generator (BioRad). Cycling conditions were as follows: 1 cycle of 95° C. (2° C./s ramp) for 10 minutes, 45 cycles of 94° C. (2° C./s ramp) for 10 seconds, 57.5° C. (2° C./s ramp) for 30 seconds, 72C (2° C./s ramp) for 1 minute, 1 cycle of 98° C. for 10 minutes, 4° C. hold. Droplets were analyzed using a QX200 droplet reader (BioRad) and QuantaSoft analysis software (BioRad) was used to acquire and analyze data.

P1:
(SEQ ID NO: 14)
AGCCCCGGGTACTCCTTGTT
F1:
(SEQ ID NO: 15)
TTCCTTGCAGGAACAGAG
R1:
(SEQ ID NO: 16)
CTGCTTGACCACCCATT
P2:
(SEQ ID NO: 17)
CCAGCAGGCCAGGTACACC
F2:
(SEQ ID NO: 18)
ACCGCCAAGGATGCAC
R2:
(SEQ ID NO: 19)
GCGGGTGGGAATGGAG

The addition of the NES to APC 11-12L.330 appeared to decrease the prevalence of nuclear indels slightly (FIG. 6), and the addition of the MVMp NS2 NES appeared to eliminate nuclear indels entirely (FIG. 7).

The MVMp NS2 NES is a highly effective addition to mitochondrial-targeted engineered meganucleases that mitigates potentially problematic nuclear off-target editing.

### General Methodology

### MTEM Expression

Total protein homogenate was prepared from flash-frozen tissues, and protein was quantified using DC protein assay (5000116, BioRad) according to manufacturer's instructions. Forty micrograms of total protein per sample was run in 10% Mini-PROTEAN TGX Stain-Free Protein Gels (4568034, BioRad), then transferred onto polyvinylidene difluoride membranes (1620260, BioRad) using the TransBlot Turbo system (1704155, BioRad) according to manufacturer's instructions. TGX Stain-Free Protein Gels allow the visualization of total protein loading through gel activation with the BioRad Chemidoc system. Blots were blocked for Ihr at RT with 5% milk. Antibodies used were mouse monoclonal Flag (F3165, Sigma-Aldrich) (1:1000), mouse monoclonal GFP (75-131, UC Davis) (1:1000), mouse monoclonal MTCO1 (ab14705, abcam) (1:1000), mouse monoclonal NDUFB8 (ab110242, abcam) (1:750), mouse monoclonal Tubulin (T9026, Sigma-Aldrich) (1:20,000), rabbit polyclonal Caspase-3 (#9662, Cell Signaling) (1:1000), and mouse monoclonal PCNA (PC10 #2586, Cell Signaling) (1:2000). Secondary antibody that was utilized was IgG horseradish peroxidase (HRP)-linked mouse (7076, Cell Signaling) (1:5000), or rabbit (7074, Cell Signaling) (1:5000). The primary antibody was incubated overnight at 4ºC, and secondary incubated for 1 h at RT. Membranes were developed with SUPERSIGNAL™ West Pico chemiluminescent substrate (34080, Thermo Scientific), and imaged in the BioRad CHEMIDOCT imager.

### DNA Extraction, Quantification by “Last-Cycle HOT” PCR, and RFLP

Total DNA was extracted from flash-frozen tissues using phenol-chloroform, and from FACS sorted cells using the NucleoSpin Tissue XS kit (740901.50, Takara). DNA concentration was determined spectrophotometrically (BioTek Synergy H1 hybrid). Levels of the m.5024C>T mutation were determined by “last-cycle hot” PCR, wherein the last cycle of the PCR is run using radioactively-labeled nucleotides. This method removes interference from heteroduplexes formed by previous melting and annealing steps by only allowing visualization of nascent amplicons. PCR amplicons were obtained with the following primers: F-5′CCACCCTAGCTATCATAAGCACA-3′ (SEQ ID NO: 12) and B-5′-AAGCAATTGATTTGCATTCAATAGATGTAGGATGAAGTCCTGCA-3′ (SEQ ID NO: 13. RFLP analysis was done by digesting amplicons with PstI-HF (R3140S, New England BioLabs), which digests the wild-type mtDNA but not the mutant mtDNA carrying the m.5024C>T point mutation. After digestion, products were run in a 12% polyacrylamide gel, and signal was detected using the Cyclone phosphor-imaging system (Perkin Elmer) and OptiQuant software Version 5.0 (Perkin Elmer).

Quantitative PCR to Determine Total mtDNA Levels

To determine total levels of mtDNA present in samples, quantitative PCR (qPCR) using TaqMan reagents (PrimeTime Std qPCR Assay, Integrated DNA Technologies) was performed as described in Bacman 2018, infra. Samples were run on a Bio-Rad CFX96/C1000 qPCR machine. Comparative cycle threshold (Ct) method was used to determine relative reads, and total mtDNA levels were determined by comparing mtDNA (ND1 and ND5) to genomic DNA (18S). The following primer/probe sets were used:

mtDNA
ND1 = Forward:
GCC TGA CCC ATA GCC ATA AT (NC_005089; mtDNA 3282-3301; SEQ ID NO: 14);
Reverse:
CGG CTG CGT ATT CTA CGT TA (mtDNA 3402-3383; SEQ ID NO: 15.
Probe:
/56-FAM/TCT CAA CCC/ZEN/TAG CAG AAA CAA CCG G/3IABKFQ/
(mtDNA 3310-3334; SEQ ID NO: 16).
NDS = Forward:
CCC ATG ACT ACC ATC AGC AAT AG (mtDNA 12432-12454; SEQ ID NO: 17);
Reverse:
TGG AAT CGG ACC AGT AGG AA (mtDNA 12533-12514; SEQ ID NO: 18).
Probe:
/5TET/AGT GCT/ZEN/GAA CTG GTG TAG GGC/3IABKFQ/ (mtDNA 12482-12458;
SEQ ID NO: 19).
Genomic DNA
18s = Forward:
GCC GCT AGA GGT GAA ATT CT (RefSeq NR_046233.2;
chr17:39984253-39984272; SEQ ID NO: 20;
Reverse:
TCG GAA CTA CGA CGG TAT CT
(RefSeq NR_046233.2; chr17:39984432-39984412; SEQ ID NO: 21).
Probe:
/5Cy5/AAG ACG GAC CAG AGC GAA AGC AT/3IAbRQSp/ (RefSeq
NR_046233.2; chr17:39984285-39984305; SEQ ID NO: 22)

### RNA Extraction, Northern Blot Analysis, and Quantification of Mt-tRNA's

RNA was isolated from flash-frozen tissues with TRIzol (Ambion) following manufacturer's standard protocols. Samples were treated with DNase (AM1907, Invitrogen) prior to spectrophotometric quantification. Northern blot analysis was done by running 4 μg total liver RNA per sample in a 1.2% agarose gel containing 20% Formaldehyde and 1×MOPS. Electrophoresis was performed in 1×MOPS solutionat 80V for 15 min followed by 120V for 2.5 hr. At this point, the gel was stained with Ethidium bromide (EtBr) to visualize total RNA loading. The gel was then washed two times for 10 min in water to remove EtBr. RNA was transferred overnight onto a nylon membrane (Amersham Hybond-NX, #RPN203T). Transcripts of interest were detected with non-radioactive biotinylated probes overnight at 50° ° C. The following day, the membrane was washed and the signal was detected with IRDye 800CW Streptavidin (926-32230, Li—COR). A biotinylated probe was used to detect mitochondrial tRNA^{Ala} by Northern blot:

(SEQ ID NO: 23)
[Btn]GACTTCATCCTACATCTATTG

The levels of mt-tRNA^{Ala} and mt-tRNA^{Val} were detected using Custom TaqMan Small RNA Assay (4398987, ThermoFisher) per manufacturer's directions. Relative levels of mt-tRNA^{Ala} were calculated by dividing Ct values by mt-tRNA^{Val} Ct values.

### Detection of Nuclear Off-Targets

This assay is a modification of GUIDE-seq, known as “oligo capture,” which is more sensitive in detecting engineered meganuclease-induced double stranded breaks [Tsai et al. (2015) Nature Biotechnology 33 (2): 187-197]. Five C57BL6J nuclear genomic sequences were identified as putative off-target sites for this nuclease, as indicated in Table 3, and tested for the presence of indels. FL83B mouse cells were electroporated with MTEM and analyzed by oligo capture×days after transformation.

TABLE 3
C57BL6J nuclear genomic sequences

| | Chromosomal | | SEQ |
|---|---|---|---|
| Site | location | Sequence | ID NO |
| Site 1 | Chromosome 2 | GTAAGGATAGTAAGTCTTCCAA | 24 |
| Site 2 | Chromosome 2 | TCAAGGATAGCACGTCTCCAAC | 25 |
| Site 3 | Chromosome 5 | TTAAGGATGGTAAGACAGTATC | 26 |
| Site 4 | Chromosome 6 | CTAAGGTTAGTAAGTATTCAAC | 27 |
| Site 5 | Chromosome 2 | TTCAGGATGGCACGTCTTCATC | 28 |

### Statistical Analysis

All data analysis was performed using GraphPad Prism 7 and 8. All statistics are presented as mean±SEM. Pairwise comparisons were performed using the unpaired two-tailed Student's t-test. Comparisons between >2 groups were done by one-way ANOVA. P-values of ≤0.05 were considered significant. N-4 mice were injected per each condition (treated vs. control, and each time point). All measurements were taken from distinct samples.

1. A mitochondria-targeting engineered meganuclease (MTEM) that binds and cleaves a recognition sequence in mitochondrial genomes of a eukaryotic cell, wherein said MTEM comprises an engineered meganuclease attached to a mitochondrial transit peptide (MTP).

2. The MTEM of claim 1, wherein said engineered meganuclease comprises a first subunit and a second subunit, wherein said first subunit binds to a first recognition half-site of said recognition sequence and comprises a first hypervariable (HVR1) region, wherein said second subunit binds to a second recognition half-site of said recognition sequence and comprises a second hypervariable (HVR2) region, and wherein said first subunit and said second subunit each comprise an amino acid sequence having at least 80% sequence identity to a sequence set forth in SEQ ID NO: 1.

3. The MTEM of any one of claim 1 or claim 2, wherein said first subunit and said second subunit each comprise an amino acid sequence having at least 80% sequence identity to residues 7-153 of SEQ ID NO: 1.

4. The MTEM of any one of claims 1-3, wherein said engineered meganuclease comprises an amino acid sequence having at least 80% sequence identity to a sequence set forth in SEQ ID NO: 2.

5. The MTEM of any one of claims 1-4, wherein said recognition sequence comprises SEQ ID NO: 3.

6. The MTEM of any one of claims 1-5, wherein said HVR1 region comprises an amino acid sequence having at least 80% sequence identity to an amino acid sequence corresponding to residues 215-270 of SEQ ID NO: 4.

7. The MTEM of any one of claims 1-6, wherein said HVR1 region comprises one or more residues corresponding to residues 215, 217, 219, 221, 223, 224, 229, 231, 233, 235, 237, 259, 261, 266, and 268 of SEQ ID NO: 4.

8. The MTEM of any one of claims 1-7, wherein said HVR1 region comprises residues 215-270 of SEQ ID NO: 4.

9. The MTEM of any one of claims 1-8, wherein said first subunit comprises an amino acid sequence having at least 80% sequence identity to residues 198-344 of SEQ ID NO: 4.

10. The MTEM of any one of claims 1-9, wherein said first subunit comprises a residue corresponding to residue 271 of SEQ ID NO: 4.

11. The MTEM of any one of claims 1-10, wherein said first subunit comprises residues 198-344 of SEQ ID NO: 4.

12. The MTEM of any one of claims 1-11, wherein said HVR2 region comprises an amino acid sequence having at least 80% sequence identity to an amino acid sequence corresponding to residues 24-79 of SEQ ID NO: 4.

13. The MTEM of any one of claims 1-12, wherein said HVR2 region comprises one or more residues corresponding to residues 24, 26, 28, 30, 32, 33, 38, 40, 42, 44, 46, 68, 70, 75, and 77 of SEQ ID NO: 4.

14. The MTEM of any one of claims 1-13, wherein said HVR2 region comprises a residue corresponding to residue 36 of SEQ ID NO: 4.

15. The MTEM of any one of claims 1-14, wherein said HVR2 region comprises residues 24-79 of SEQ ID NO: 4.

16. The MTEM of any one of claims 1-15, wherein said second subunit comprises an amino acid sequence having at least 80% sequence identity to residues 7-153 of SEQ ID NO: 4.

17. The MTEM of any one of claims 1-16, wherein said second subunit comprises residues 7-153 of SEQ ID NO: 4.

18. The MTEM of any one of claims 1-17, wherein said engineered meganuclease is a single-chain meganuclease comprising a linker, wherein said linker covalently joins said first subunit and said second subunit.

19. The MTEM of any one of claims 1-18, wherein said engineered meganuclease comprises an amino acid sequence having at least 80% sequence identity to SEQ ID NO: 4.

20. The MTEM of any one of claims 1-19, wherein said engineered meganuclease comprises an amino acid sequence of SEQ ID NO: 4.

21. The MTEM of any one of claims 1-20, wherein said engineered meganuclease is encoded by a nucleic sequence having at least 80% sequence identity to a nucleic acid sequence of SEQ ID NO: 5.

22. The MTEM of any one of claims 1-21, wherein said engineered meganuclease is encoded by a nucleic acid sequence of SEQ ID NO: 5.

23. The MTEM of any one of claims 1-22, wherein said MTP comprises an amino acid sequence having at least 80% sequence identity to a sequence set forth in any one of SEQ ID NOs: 6-8.

24. The MTEM of any one of claims 1-23, wherein said MTP comprises an amino acid sequence set forth in any one of SEQ ID NOs: 6-8.

25. The MTEM of any one of claims 1-24, wherein said MTP is attached to the C-terminus of said engineered meganuclease.

26. The MTEM of any one of claims 1-24, wherein said MTP is attached to the N-terminus of said engineered meganuclease.

27. The MTEM of any one of claims 1-26, wherein said MTP is fused to said engineered meganuclease.

28. The MTEM of any one of claims 1-26, wherein said MTP is attached to said engineered meganuclease by a polypeptide linker.

29. The MTEM of any one of claims 1-24, wherein said engineered meganuclease is attached to a first MTP and a second MTP.

30. The MTEM of claim 29, wherein said first MTP and/or said second MTP comprises an amino acid sequence having at least 80% sequence identity to a sequence set forth in any one of SEQ ID NOs: 6-8.

31. The MTEM of claim 29 or claim 30, wherein said first MTP and/or said second MTP comprises an amino acid sequence set forth in any one of SEQ ID NOs: 6-8.

32. The MTEM of any one of claims 29-31, wherein said first MTP and said second MTP are identical.

33. The MTEM of any one of claims 29-31, wherein said first MTP and said second MTP are not identical.

34. The MTEM of any one of claims 29-33, wherein said first MTP and/or said second MTP is fused to said engineered meganuclease.

35. The MTEM of any one of claims 29-33, wherein said first MTP and/or said second MTP is attached to said engineered meganuclease by a polypeptide linker.

36. The MTEM of any one of claims 1-35, wherein said MTEM is attached to a nuclear export sequence (NES).

37. The MTEM of any one of claims 1-36, wherein said NES comprises an amino acid sequence having at least 80% sequence identity to a sequence set forth in SEQ ID NO: 9 or 10.

38. The MTEM of any one of claims 1-37, wherein said NES comprises an amino acid sequence set forth in SEQ ID NO: 9 or 10.

39. The MTEM of any one of claims 1-38, wherein said NES is attached at the N-terminus of said MTEM.

40. The MTEM of any one of claims 1-38, wherein said NES is attached at the C-terminus of said MTEM.

41. The MTEM of any one of claims 1-40, wherein said NES is fused to said MTEM.

42. The MTEM of any one of claims 1-40, wherein said NES is attached to said MTEM by a polypeptide linker.

43. The MTEM of any one of claims 1-36, wherein said MTEM comprises a first NES and a second NES.

44. The MTEM of claim 43, wherein said first NES is attached at the N-terminus of said MTEM, and wherein said second NES is attached at the C-terminus of said MTEM.

45. The MTEM of claim 43 or claim 44, wherein said first NES and/or said second NES comprises an amino acid sequence having at least 80% sequence identity to a sequence set forth in SEQ ID NO: 9 or 10.

46. The MTEM of any one of claims 43-45, wherein said first NES and/or said second NES comprises an amino acid sequence set forth in SEQ ID NO: 9 or 10.

47. The MTEM of any one of claims 43-46, wherein said first NES and said second NES are identical.

48. The MTEM of any one of claims 43-46, wherein said first NES and said second NES are not identical.

49. The MTEM of any one of claims 43-48, wherein said first NES and/or said second NES is fused to said MTEM.

50. The MTEM of any one of claims 43-48, wherein said first NES and/or said second NES is attached to said MTEM.

51. A polynucleotide comprising a nucleic acid sequence encoding said MTEM of any one of claims 1-50.

52. The polynucleotide of claim 51, wherein said polynucleotide is an mRNA.

53. A recombinant DNA construct comprising a polynucleotide comprising a nucleic acid sequence encoding said MTEM of any one of claims 1-50.

54. The recombinant DNA construct of claim 53, wherein said recombinant DNA construct encodes a recombinant virus comprising said polynucleotide.

55. The recombinant DNA construct of claim 54, wherein said recombinant virus is a recombinant adenovirus, a recombinant lentivirus, a recombinant retrovirus, or a recombinant adeno-associated virus (AAV).

56. The recombinant DNA construct of claim 54 or claim 55, wherein said recombinant virus is a recombinant AAV.

57. The recombinant DNA construct of any one of claims 54-56, wherein said polynucleotide comprises a promoter operably linked to said nucleic acid sequence encoding said MTEM.

58. The recombinant DNA construct of claim 57, wherein said promoter is a constitutive promoter or a tissue-specific promoter.

59. The recombinant DNA construct of claim 58, wherein said constitutive promoter is a CMV promoter, a CAG promoter, an EF1 alpha promoter, or a UbC promoter, or wherein said tissue-specific promoter is a neuron-specific promoter, an astrocyte-specific promoter, a microglia-specific promoter, a muscle-specific promoter, a skeletal muscle-specific promoter, a myotube-specific promoter, muscle satellite cell-specific promoter, a cardiomyocyte-specific promoter, an eye-specific promoter, a retina-specific promoter, a retinal ganglion cell-specific promoter, a retinal pigmentary epithelium-specific promoter, a leukocyte-specific promoter, a progenitor cell-specific promoter, a blood progenitor cell-specific promoter, a pancreas-specific promoter, a pancreatic beta cell-specific promoter, an endothelial cell-specific promoter, an inner ear hair cell-specific promoter, a bone marrow cell-specific promoter, or a kidney-specific promoter.

60. A recombinant virus comprising a polynucleotide comprising a nucleic acid sequence encoding said MTEM of any one of claims 1-50.

61. The recombinant virus of claim 60, wherein said recombinant virus is a recombinant adenovirus, a recombinant lentivirus, a recombinant retrovirus, or a recombinant adeno-associated virus (AAV).

62. The recombinant virus of claim 60 or claim 61, wherein said recombinant virus is a recombinant AAV.

63. The recombinant virus of any one of claims 60-62, wherein said polynucleotide comprises a promoter operably linked to said nucleic acid sequence encoding said MTEM.

64. The recombinant virus of claim 63, wherein said promoter is a constitutive promoter or a tissue-specific promoter.

65. The recombinant virus of claim 64, wherein said constitutive promoter is a CMV promoter, a CAG promoter, an EF1 alpha promoter, or a UbC promoter, or wherein said tissue-specific promoter is a neuron-specific promoter, an astrocyte-specific promoter, a microglia-specific promoter, a muscle-specific promoter, a skeletal muscle-specific promoter, a myotube-specific promoter, muscle satellite cell-specific promoter, a cardiomyocyte-specific promoter, an eye-specific promoter, a retina-specific promoter, a retinal ganglion cell-specific promoter, a retinal pigmentary epithelium-specific promoter, a leukocyte-specific promoter, a progenitor cell-specific promoter, a blood progenitor cell-specific promoter, a pancreas-specific promoter, a pancreatic beta cell-specific promoter, an endothelial cell-specific promoter, an inner ear hair cell-specific promoter, a bone marrow cell-specific promoter, or a kidney-specific promoter.

66. A lipid nanoparticle composition comprising lipid nanoparticles comprising a polynucleotide, wherein said polynucleotide comprises a nucleic acid sequence encoding said MTEM of any one of claims 1-50.

67. The lipid nanoparticle composition of claim 66, wherein said polynucleotide is an mRNA.

68. A pharmaceutical composition comprising a pharmaceutically acceptable carrier and said MTEM of any one of claims 1-50.

69. A pharmaceutical composition comprising a pharmaceutically acceptable carrier and said polynucleotide of claim 51 or claim 52.

70. A pharmaceutical composition comprising a pharmaceutically acceptable carrier and said recombinant DNA construct of any one of claims 53-59.

71. A pharmaceutical composition comprising a pharmaceutically acceptable carrier and said recombinant virus of any one of claims 60-65.

72. A pharmaceutical composition comprising a pharmaceutically acceptable carrier and said lipid nanoparticle composition of claim 66 or claim 67.

73. A genetically-modified eukaryotic cell comprising said polynucleotide of claim 51 or claim 52.

74. The genetically-modified eukaryotic cell of claim 73, wherein said genetically-modified eukaryotic cell is a genetically-modified mammalian cell.

75. The genetically-modified eukaryotic cell of claim 73, wherein said genetically-modified eukaryotic cell is a genetically-modified human cell.

76. The genetically-modified eukaryotic cell of claim 73, wherein said genetically-modified eukaryotic cell is a genetically-modified plant cell.

77. A method for producing a genetically-modified eukaryotic cell, said method comprising introducing into a eukaryotic cell:
(a) a polynucleotide comprising a nucleic acid sequence encoding said MTEM of any one of claims 1-50, wherein said MTEM is expressed in said eukaryotic cell; or
(b) said MTEM of any one of claims 1-50;
wherein said MTEM produces a cleavage site at said recognition sequence in mitochondrial genomes of said eukaryotic cell.

78. The method of claim 77, wherein said cleavage site is repaired by non-homologous end joining, such that said recognition sequence comprises an insertion or deletion.

79. The method of claim 77, wherein said mitochondrial genomes comprising said recognition sequence are degraded in said genetically-modified eukaryotic cell.

80. The method of claim 79, wherein said mitochondrial genomes are mutant mitochondrial genomes.

81. The method of claim 79 or claim 80, wherein about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99% of mutant mitochondrial genomes comprising said recognition sequence are degraded in said genetically-modified eukaryotic cell.

82. The method of any one of claims 79-81, wherein the ratio of wild-type mitochondrial genomes to mutant mitochondrial genomes comprising said recognition sequence increases in said genetically-modified eukaryotic cell.

83. The method of any one of claims 79-82, wherein said ratio increases to about 5:95, about 10:90, about 15:85, about 20:80, about 25:75, about 30:70, about 35:65, about 40:60, about 45:55, about 50:50, about 55:45, about 60:40, about 65:35, about 70:30, about 75:25, about 80:20, about 85:15, about 90:10, about 95:5, about 20:1, about 50:1, about 100:1, about 150:1, about 200:1, about 250:1, about 300:1, about 350:1, about 400:1, about 450:1, about 500:1, about 550:1, about 600:1, about 650:1, about 700:1, about 750:1, about 800:1, about 850:1, about 900:1, about 950:1, about 1000:1, or more.

84. The method of any one of claims 79-83, wherein the percentage of wild-type mitochondrial genomes in said genetically-modified eukaryotic cell is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more, of the total mitochondrial genomes in said genetically-modified eukaryotic cell.

85. The method of any one of claims 79-84, wherein the percentage of mutant mitochondrial genomes comprising said recognition sequence in said genetically-modified eukaryotic cell decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more.

86. The method of any one of claims 79-85, wherein cellular respiration in said genetically-modified eukaryotic cell increases by about 30%, about 35%, about 40%, about 45%, about 50%, about 55%, about 60%, about 65%, about 70%, about 75%, about 80%, about 85%, about 90%, about 95%, about 100%, or more.

87. The method of any one of claims 79-86, wherein cellular respiration in said genetically-modified eukaryotic cell increases by about 30-40%, about 40-50%, about 50-60%, about 60-70%, about 70-80%, about 80-90%, about 90-100%, or more.

88. A method for producing a population of eukaryotic cells comprising a plurality of genetically-modified cells, said method comprising introducing into a plurality of eukaryotic cells in said population: (a) a polynucleotide comprising a nucleic acid sequence encoding said MTEM of any one of claims 1-50, wherein said MTEM is expressed in said plurality of eukaryotic cells; or (b) said MTEM of any one of claims 1-50; wherein said MTEM produces a cleavage site at said recognition sequence in mitochondrial genomes of said plurality of eukaryotic cells.

89. The method of claim 88, wherein said cleavage site is repaired by non-homologous end joining, such that said recognition sequence comprises an insertion or deletion.

90. The method of claim 88, wherein said mitochondrial genomes comprising said recognition sequence are degraded in said plurality of genetically-modified eukaryotic cells.

91. The method of claim 90, wherein said mitochondrial genomes are mutant mitochondrial genomes.

92. The method of claim 90 or claim 91, wherein about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99% of mutant mitochondrial genomes comprising said recognition sequence are degraded in said plurality of genetically-modified eukaryotic cells.

93. The method of any one of claims 90-92, wherein the ratio of wild-type mitochondrial genomes to mutant mitochondrial genomes comprising said recognition sequence increases in said plurality of genetically-modified eukaryotic cells.

94. The method of any one of claims 90-93, wherein the ratio of wild-type mitochondrial genomes to mutant mitochondrial genomes comprising said recognition sequence increases in said population of eukaryotic cells.

95. The method of any one of claims 90-94, wherein said ratio increases to about 5:95, about 10:90, about 15:85, about 20:80, about 25:75, about 30:70, about 35:65, about 40:60, about 45:55, about 50:50, about 55:45, about 60:40, about 65:35, about 70:30, about 75:25, about 80:20, about 85:15, about 90:10, about 95:5, about 20:1, about 50:1, about 100:1, about 150:1, about 200:1, about 250:1, about 300:1, about 350:1, about 400:1, about 450:1, about 500:1, about 550:1, about 600:1, about 650:1, about 700:1, about 750:1, about 800:1, about 850:1, about 900:1, about 950:1, about 1000:1, or more.

96. The method of any one of claims 90-95, wherein the percentage of wild-type mitochondrial genomes in said plurality of genetically-modified eukaryotic cells increases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more.

97. The method of any one of claims 90-96, wherein the percentage of wild-type mitochondrial genomes in said population of eukaryotic cells increases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more.

98. The method of any one of claims 90-97, wherein the percentage of mutant mitochondrial genomes comprising said recognition sequence in said plurality of genetically-modified eukaryotic cells decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more.

99. The method of any one of claims 90-98, wherein the percentage of mutant mitochondrial genomes comprising said recognition sequence in said population of eukaryotic cells decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more.

100. The method of any one of claims 90-99, wherein cellular respiration in said plurality of genetically-modified eukaryotic cells increases by about 30%, about 35%, about 40%, about 45%, about 50%, about 55%, about 60%, about 65%, about 70%, about 75%, about 80%, about 85%, about 90%, about 95%, about 100%, or more.

101. The method of any one of claims 90-100, wherein cellular respiration in said plurality of genetically-modified eukaryotic cells increases by about 30-40%, about 40-50%, about 50-60%, about 60-70%, about 70-80%, about 80-90%, about 90-100%, or more.

102. The method of any one of claims 90-101, wherein cellular respiration in said population of eukaryotic cells increases by about 30%, about 35%, about 40%, about 45%, about 50%, about 55%, about 60%, about 65%, about 70%, about 75%, about 80%, about 85%, about 90%, about 95%, about 100%, or more.

103. The method of any one of claims 90-102, wherein cellular respiration in said population of eukaryotic cells increases by about 30-40%, about 40-50%, about 50-60%, about 60-70%, about 70-80%, about 80-90%, about 90-100%, or more.

104. The method of any one of claims 77-103, wherein said method is performed in vivo.

105. The method of any one of claims 77-103, wherein said method is performed in vitro.

106. The method of any one of claims 77-105, wherein said polynucleotide is an mRNA.

107. The method of claim 106, wherein said polynucleotide is said mRNA of claim 52.

108. The method of any one of claims 77-105, wherein said polynucleotide is a recombinant DNA construct.

109. The method of claim 108, wherein said polynucleotide is said recombinant DNA construct of any one of claims 53-59.

110. The method of any one of claims 77-105, wherein said polynucleotide is introduced into said eukaryotic cell by a lipid nanoparticle.

111. The method of any one of claims 77-105, wherein said polynucleotide is introduced into said eukaryotic cell by a recombinant virus.

112. The method of claim 111, wherein said recombinant virus is said recombinant virus of any one of claims 60-65.

113. The method of claim 111 or claim 112, wherein said recombinant virus is a recombinant AAV.

114. The method of any one of claims 77-113, wherein said polynucleotide comprises a promoter operably linked to said nucleic acid sequence encoding said MTEM.

115. The method of claim 114, wherein said promoter is a constitutive promoter or a tissue-specific promoter.

116. The method of claim 115, wherein said constitutive promoter is a CMV promoter, a CAG promoter, an EF1 alpha promoter, or a UbC promoter, or wherein said tissue-specific promoter is a neuron-specific promoter, an astrocyte-specific promoter, a microglia-specific promoter, a muscle-specific promoter, a skeletal muscle-specific promoter, a myotube-specific promoter, muscle satellite cell-specific promoter, a cardiomyocyte-specific promoter, an eye-specific promoter, a retina-specific promoter, a retinal ganglion cell-specific promoter, a retinal pigmentary epithelium-specific promoter, a leukocyte-specific promoter, a progenitor cell-specific promoter, a blood progenitor cell-specific promoter, a pancreas-specific promoter, a pancreatic beta cell-specific promoter, an endothelial cell-specific promoter, an inner ear hair cell-specific promoter, a bone marrow cell-specific promoter, or a kidney-specific promoter.

117. The method of any one of claims 77-116, wherein said eukaryotic cell is a mammalian cell.

118. The method of any one of claims 77-117, wherein said eukaryotic cell is a human cell.

119. The method of claim 118, wherein said eukaryotic cell is a neuron, an astrocyte, a microglia cell, a muscle cell, a skeletal muscle cell, a myotube cell, a muscle satellite cell, a cardiomyocyte, a cell of the eye, a retinal cell, a retinal ganglion cell, a retinal pigmentary epithelium cell, a leukocyte, a progenitor cell, a blood progenitor cell, a pancreas cell, a pancreatic beta cell, an endothelial cell, an inner ear hair cell, a bone marrow cell, or a kidney cell.

120. The method of any one of claims 77-116, wherein said eukaryotic cell is a plant cell.

121. A genetically-modified eukaryotic cell, or a population of genetically-modified eukaryotic cells, produced by the method of any one of claims 77-120.

122. A method for degrading mutant mitochondrial genomes in a target cell in a subject, or in a population of target cells in a subject, said method comprising delivering to said target cell or said population of target cells:
(a) a polynucleotide comprising a nucleic acid sequence encoding said MTEM of any one of claims 1-50, wherein said MTEM is expressed in said target cell or said population of target cells; or
(b) said MTEM of any one of claims 1-50;
wherein said MTEM produces a cleavage site in said mutant mitochondrial genomes at a recognition sequence, and wherein said mutant mitochondrial genomes are degraded.

123. The method of claim 122, wherein said subject is a mammal.

124. The method of claim 122 or claim 123 wherein said subject is a human.

125. The method of any one of claims 122-124, wherein said target cell is a neuron, an astrocyte, a microglia cell, a muscle cell, a skeletal muscle cell, a myotube cell, a muscle satellite cell, a cardiomyocyte, a cell of the eye, a retinal cell, a retinal ganglion cell, a retinal pigmentary epithelium cell, a leukocyte, a progenitor cell, a blood progenitor cell, a pancreas cell, a pancreatic beta cell, an endothelial cell, an inner ear hair cell, or a kidney cell, or wherein said population of target cells is a population of neurons, astrocytes, microglia cells, muscle cells, skeletal muscle cells, myotube cells, muscle satellite cells, cardiomyocytes, cells of the eye, retinal cells, retinal ganglion cells, retinal pigmentary epithelium cells, leukocytes, progenitor cells, blood progenitor cells, pancreas cells, pancreatic beta cells, endothelial cells, inner ear hair cells, or kidney cells.

126. The method of any one of claims 122-125, wherein said polynucleotide is an mRNA.

127. The method of claim 126, wherein said polynucleotide is said mRNA of claim 52.

128. The method of any one of claims 122-125, wherein said polynucleotide is a recombinant DNA construct.

129. The method of claim 128, wherein said polynucleotide is said recombinant DNA construct of any one of claims 53-59.

130. The method of any one of claims 122-125, wherein said polynucleotide is delivered to said target cell, or said population of target cells, by a lipid nanoparticle.

131. The method of any one of claims 122-125, wherein said polynucleotide is delivered to said target cell, or said population of target cells, by a recombinant virus.

132. The method of claim 131, wherein said recombinant virus is said recombinant virus of any one of claims 60-65.

133. The method of claim 130 or claim 131, wherein said recombinant virus is a recombinant AAV.

134. The method of any one of claims 122-133, wherein said polynucleotide comprises a promoter operably linked to said nucleic acid sequence encoding said MTEM.

135. The method of claim 134, wherein said promoter is a constitutive promoter or a tissue-specific promoter.

136. The method of claim 135, wherein said constitutive promoter is a CMV promoter, a CAG promoter, an EF1 alpha promoter, or a UbC promoter, or wherein said tissue-specific promoter is a neuron-specific promoter, an astrocyte-specific promoter, a microglia-specific promoter, a muscle-specific promoter, a skeletal muscle-specific promoter, a myotube-specific promoter, muscle satellite cell-specific promoter, a cardiomyocyte-specific promoter, an eye-specific promoter, a retina-specific promoter, a retinal ganglion cell-specific promoter, a retinal pigmentary epithelium-specific promoter, a leukocyte-specific promoter, a progenitor cell-specific promoter, a blood progenitor cell-specific promoter, a pancreas-specific promoter, a pancreatic beta cell-specific promoter, an endothelial cell-specific promoter, an inner ear hair cell-specific promoter, a bone marrow cell-specific promoter, or a kidney-specific promoter.

137. The method of any one of claims 122-136, wherein about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99% of mutant mitochondrial genomes comprising said recognition sequence are degraded in said target cell or said population of said target cells.

138. The method of any one of claims 122-137, wherein the ratio of wild-type mitochondrial genomes to mutant mitochondrial genomes comprising said recognition sequence increases in said target cell or said population of target cells.

139. The method of any one of claims 122-138, wherein said ratio increases to about 5:95, about 10:90, about 15:85, about 20:80, about 25:75, about 30:70, about 35:65, about 40:60, about 45:55, about 50:50, about 55:45, about 60:40, about 65:35, about 70:30, about 75:25, about 80:20, about 85:15, about 90:10, about 95:5, about 20:1, about 50:1, about 100:1, about 150:1, about 200:1, about 250:1, about 300:1, about 350:1, about 400:1, about 450:1, about 500:1, about 550:1, about 600:1, about 650:1, about 700:1, about 750:1, about 800:1, about 850:1, about 900:1, about 950:1, about 1000:1, or more.

140. The method of any one of claims 122-139, wherein the percentage of wild-type mitochondrial genomes in said target cell or said population of target cells is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more, of the total mitochondrial genomes in said target cell or said population of target cells.

141. The method of any one of claims 122-140, wherein the percentage of mutant mitochondrial genomes comprising said recognition sequence in said genetically-modified eukaryotic cell decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more.

142. The method of any one of claims 122-141, wherein cellular respiration in said target cell or said population of target cells increases by about 30%, about 35%, about 40%, about 45%, about 50%, about 55%, about 60%, about 65%, about 70%, about 75%, about 80%, about 85%, about 90%, about 95%, about 100%, or more.

143. The method of any one of claims 122-142, wherein cellular respiration in said target cell or said population of target cells increases by about 30-40%, about 40-50%, about 50-60%, about 60-70%, about 70-80%, about 80-90%, about 90-100%, or more.

144. A method for treating a condition associated with a mitochondrial disorder in a subject, said method comprising administering to said subject:
(a) a therapeutically-effective amount of a polynucleotide comprising a nucleic acid sequence encoding said MTEM of any one of claims 1-50, wherein said polynucleotide is delivered to a target cell, or a population of target cells, in said subject, and wherein said MTEM is expressed in said target cell or said population of target cells; or
(b) a therapeutically-effective amount of said MTEM of any one of claims 1-50, wherein said MTEM is delivered to a target cell, or a population of target cells, in said subject;
wherein said MTEM produces a cleavage site in mutant mitochondrial genomes at a recognition sequence, and wherein said mutant mitochondrial genomes are degraded.

145. The method of claim 144, wherein said method comprises administering said pharmaceutical composition of any one of claims 68-72.

146. The method of claim 144 or claim 145, wherein said subject is a mammal.

147. The method of any one of claims 144-146, wherein said subject is a human.

148. The method of any one of claims 144-147, wherein said target cell is a neuron, an astrocyte, a microglia cell, a muscle cell, a skeletal muscle cell, a myotube cell, a muscle satellite cell, a cardiomyocyte, a cell of the eye, a retinal cell, a retinal ganglion cell, a retinal pigmentary epithelium cell, a leukocyte, a progenitor cell, a blood progenitor cell, a pancreas cell, a pancreatic beta cell, an endothelial cell, an inner ear hair cell, or a kidney cell, or wherein said population of target cells is a population of neurons, astrocytes, microglia cells, muscle cells, skeletal muscle cells, myotube cells, muscle satellite cells, cardiomyocytes, cells of the eye, retinal cells, retinal ganglion cells, retinal pigmentary epithelium cells, leukocytes, progenitor cells, blood progenitor cells, pancreas cells, pancreatic beta cells, endothelial cells, inner ear hair cells, or kidney cells.

149. The method of any one of claims 144-148, wherein said condition is a condition of the muscle, heart, central nervous system, eye, bone marrow, kidney, pancreas, white blood cells, blood vessels, or inner ear.

150. The method of any one of claims 144-149, wherein said condition is Pearson Syndrome, Progressive external Ophthalmoplegia, Kearns-Sayre Syndrome (KSS), Myoclonic Epilepsy with Ragged Red Fibers (MERRF), Neuropathy, Ataxia, Retinitis Pigmentosa (NARP), Leber Hereditary Optic Neuropathy (LHON), Chronic Progressive External Ophthalmoplegia (CPEO), Maternally Inherited Leigh Syndrome (MILS), Maternally Inherited Diabetes and Deafness (MIDD), or mitochondria disorders with overlap symptoms.

151. The method of any one of claims 144-150, wherein said polynucleotide is an mRNA.

152. The method of claim 151, wherein said polynucleotide is said mRNA of claim 52.

153. The method of any one of claims 144-150, wherein said polynucleotide is a recombinant DNA construct.

154. The method of claim 153, wherein said polynucleotide is said recombinant DNA construct of any one of claims 53-59.

155. The method of any one of claims 144-150, wherein said polynucleotide is delivered to said target cell, or said population of target cells, by a lipid nanoparticle.

156. The method of any one of claims 144-150, wherein said polynucleotide is delivered to said target cell, or said population of target cells, by a recombinant virus.

157. The method of claim 156, wherein said recombinant virus is said recombinant virus of any one of claims 60-65.

158. The method of claim 156 or claim 157, wherein said recombinant virus is a recombinant AAV.

159. The method of any one of claims 144-158, wherein said polynucleotide comprises a promoter operably linked to said nucleic acid sequence encoding said MTEM.

160. The method of claim 159, wherein said promoter is a tissue-specific promoter.

161. The method of any one of claims 144-160, wherein about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99% of mutant mitochondrial genomes comprising said recognition sequence are degraded in said target cell or said population of said target cells.

162. The method of any one of claims 144-161, wherein the ratio of wild-type mitochondrial genomes to mutant mitochondrial genomes comprising said recognition sequence increases in said target cell or said population of target cells.

163. The method of any one of claims 144-162, wherein said ratio increases to about 5:95, about 10:90, about 15:85, about 20:80, about 25:75, about 30:70, about 35:65, about 40:60, about 45:55, about 50:50, about 55:45, about 60:40, about 65:35, about 70:30, about 75:25, about 80:20, about 85:15, about 90:10, about 95:5, about 20:1, about 50:1, about 100:1, about 150:1, about 200:1, about 250:1, about 300:1, about 350:1, about 400:1, about 450:1, about 500:1, about 550:1, about 600:1, about 650:1, about 700:1, about 750:1, about 800:1, about 850:1, about 900:1, about 950:1, about 1000:1, or more.

164. The method of any one of claims 144-163, wherein the percentage of wild-type mitochondrial genomes in said target cell or said population of target cells is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more, of the total mitochondrial genomes in said target cell or said population of target cells.

165. The method of any one of claims 144-164, wherein the percentage of mutant mitochondrial genomes comprising said recognition sequence in said genetically-modified eukaryotic cell decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more.

166. The method of any one of claims 144-165, wherein cellular respiration in said target cell or said population of target cells increases by about 30%, about 35%, about 40%, about 45%, about 50%, about 55%, about 60%, about 65%, about 70%, about 75%, about 80%, about 85%, about 90%, about 95%, about 100%, or more.

167. The method of any one of claims 144-166, wherein cellular respiration in said target cell or said population of target cells increases by about 30-40%, about 40-50%, about 50-60%, about 60-70%, about 70-80%, about 80-90%, about 90-100%, or more.
